SEQUENCE LISTING (4 sequences)

SEQ ID NO: 1 - DNA, 723 bp, Artificial
  artificial antibody chain
atggaagccccagcgcagcttctcttcctcctgctactctggctcccagataccactggagaaatagtgatgacgcagtctccagccaccctgtctgtgtctccaggggaaagagccaccctctcctgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 1389 bp, Artificial
  artificial antibody chain
atgggatggacctggatctttattttaatcctgtcggtaactacaggtgtccactctgaggtccaactgctgcagtctggacctgagctggagaagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 3 - protein, 240 aa, Artificial
  artificial antibody chain
MEAPAQLLFLLLLWLPDTTGEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 462 aa, Artificial
  artificial antibody chain
MGWTWIFILILSVTTGVHSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0295339 A1
(43) Pub. Date: Sep. 21, 2023

(54) METHOD FOR TREATING A GD2-POSITIVE CANCER

(71) Applicant: EUSA PHARMA (UK) LIMITED, Hertfordshire (GB)

(72) Inventors: Holger Nikolaus LODE, Greifswald (DE); Ruth LADENSTEIN, Wien (AT)

(73) Assignee: EUSA PHARMA (UK) LIMITED, Hertfordshire (GB)

(21) Appl. No.: 18/004,339

(22) PCT Filed: Jul. 6, 2021
(86) PCT No.: PCT/GB2021/051721

§ 371 Date: Jan. 5, 2023

(30) Foreign Application Priority Data
Jul. 6, 2020 (EP) 20305766.6

(51) Int. Cl. C07K 16/30; A61K 39/395; A61K 33/243; A61K 31/365; A61K 31/475; A61K 31/675; A61K 31/655; A61K 31/704; A61K 31/282; A61K 31/664; A61P 35/00

(52) U.S. Cl. CPC C07K 16/3084; A61K 39/39558; A61K 33/243; A61K 31/365; A61K 31/475; A61K 31/675; A61K 31/655; A61K 31/704; A61K 31/282; A61K 31/664; A61P 35/00; C07K 2317/732; C07K 2317/734; C07K 2317/24; A61K 2039/505

(57) ABSTRACT

A method of treating a newly diagnosed neuroblastoma in a patient by administering chimeric anti-GD2 antibody dinutuximab beta to the patient in combination with induction chemotherapy. The chimeric anti-GD2 antibody dinutuximab beta is administered at a cumulative dose of up to 500 mg/m², and/or in a dose of up to 100 mg/m² per cycle during one or more cycles of induction chemotherapy, and/or at a treatment density of up to 5 mg/m²/day.

### FIELD OF THE INVENTION

The present invention relates to induction therapy for treating newly diagnosed neuroblastoma, particularly involving induction chemotherapy and therapy with an anti-GD2 antibody.

### BACKGROUND TO THE INVENTION

Neuroblastoma, after brain cancer, is the most frequent solid cancer in children under 5 years of age. In high-risk neuroblastoma, more than half of the patients receiving standard therapy have a relapse and ultimately die from the disease. Approximately 90% of cases occur between ages 0 to 6 years. The worldwide incidence in industrialized countries is around 2000 cases per year.

Over the last 4 decades, different chemotherapy regimens have been evaluated in this treatment setting by academic cooperative groups (GPOH, International Society of Pediatric Oncology European Neuroblastoma [SIOPEN] research network, and the Children's Oncology Group [COG]) with increasing intensity and different combinations of conventional chemotherapeutics. Most induction chemotherapy regimens include platinum compounds (cisplatin and/or carboplatin), cyclophosphamide, etoposide, and vincristine, topoisomerase inhibitors (topotecan), and anthracyclines.

Multiple reports have demonstrated that achieving a complete response (CR) after induction therapy is one of the most powerful predictors of outcome. This was shown in a study by the European Bone Marrow Transplantation registry where achieving CR before high-dose chemotherapy was an independent predictor of event-free survival (EFS) and overall survival (OS) in multivariable analysis (Ladenstein R et al, Bone Marrow Transplant. 2008; 41 Suppl 2:S118-27).

Monoclonal antibodies against specific antigens are increasingly being used in oncology. The entirely different mode of action compared to cytotoxic therapies have made them a valuable asset as is shown by forerunners like trastuzumab, cetuximab, bevacizumab, rituximab, and others in various oncology indications. The disialoganglioside GD2 is a glycosphingolipid expressed primarily on the cell surface. GD2 expression in normal tissues is rare and primarily restricted to the central nervous system (CNS), peripheral nerves, and melanocytes. In cancerous cells, GD2 is uniformly expressed in neuroblastomas and most melanomas and to a variable degree in bone and soft-tissue sarcomas, small-cell lung cancer, renal cell carcinoma, and brain tumors (Navid et al., Curr Cancer Drug Targets 2010). GD2 is also expressed in Ewing sarcoma (Kailayangiri S et al., Br J Cancer. 2012; 106(6):1123-1133. doi:10.1038/bjc.2012.57), breast cancer (Orsi G et al. Oncotarget 2017: 8:31592-31600), desmoplastic small round cell tumor (Dobrenkov K et al. Pediatr Blood Cancer. 2016: 63:1780-1785) and retinoblastoma (Fleurence J et al. Journal of Immunology Research, Volume 2017, Article ID 5604891, https://doi.org/10.1155/201715604891). Because of the relatively tumor-selective expression combined with its presence on the cell surface, GD2 represents a promising target for antibody-based cancer immunotherapy.

The molecular properties of anti-GD2 monoclonal antibodies which have been developed clinically, and are derived from 14G2a or 3F8, are described in Sterner et at 2017 Cell Reports 20, 1681-1691. Antibody 14G2a has been developed as chimeric (murine/human) forms known as ch14.18, in particular, dinutuximab beta (Qarziba®) and dinutuximab (Unituxin®). An independently generated murine antibody 3F8 has been humanized as naxitamab. According to Sterner (Cell Reports, supra), humanized 3F8 has an apparent kD of 7.7 nM for binding to GD2, a binding preference for GD2 versus the related glycan structure GT2 of 1500, and a binding preference for GD2 versus the related glycan structure GQ2 of 200, whereas Unituxin has an apparent kD of 60 nM for binding to GD2, a binding preference for GD2 versus GT2 of >5000 and a binding preference for GD2 versus GQ2 of 1000. These differences reflect the different binding regions of the antibodies. Dinutuximab beta, which is produced in Chinese Hamster Ovary (CHO) cells, and Unituxin, which is produced in SP2/0 murine hybridoma cells, differ in their glycosylation patterns. Dinutuximab beta has a single N-linked glycosylation site (Asn 293), and mass spectrometry analysis revealed that the heavy chain contains the typical IgG diantennary fucosylated N-glycans with 0, 1, or 2 galactose residues, with a smaller fraction of glycans with sialic acid and oligomannose residues, and no Gal-α-1,3 Gal, typical for IgG expression in CHO cells (European Public Assessment Report (EPAR) of the Committee for Medicinal Products for Human Use (CHMP) of the European Medicines Agency (EMA) for Dinutuximab beta Apeiron (EMA/263814/2017, 23 Mar. 2017). A humanized version of ch14.18 known as hu14.18K322A is described in WO2005/070967 and has a point mutation in the Fc region in order to reduce complement-dependent cytotoxicity (CDC) but still maintain antibody-dependent cellular cytotoxicity (ADCC). The reduction in CDC is considered to result in reduced pain associated with the antibody treatment. However, it is shown in U.S. Pat. No. 9,777,068B2 that the cytolysis capacity of an anti-GD2 antibody as measured by a CDC assay is essential for the anti-tumour effect.

Dinutuximab beta, also referred to as ch14.18/CHO or APN311 is licensed in the European Union (EU) subject to additional monitoring as Qarziba® and is administered at 10 mg/m²/day as an 8-hour or 24-hour infusion (Dinutuximab Beta Investigator's Brochure. Version 3.0 dated 14 May 2019). Dinutuximab beta is indicated for the treatment of high-risk neuroblastoma in patients aged 12 months and above, who have previously received induction chemotherapy and achieved at least a partial response, followed by myeloablative therapy and stem cell transplantation, as well as patients with history of relapsed or refractory neuroblastoma, with or without residual disease. Dinutuximab beta is also authorised as a medicinal product in Australia and Israel. Various patents cover methods of using Dinutuximab beta, particularly U.S. Pat. No. 9,777,068 B2 which discloses a continuous intravenous infusion regimen which reduces the side-effect of pain; and U.S. Pat. No. 9,840,566 B2 and U.S. Pat. No. 10,294,305 B2 which disclose treatment regimens in which IL-2 is not administered in the same treatment cycle or overall treatment period.

Toxicity profiles of chemotherapy drugs and monoclonal antibodies may overlap, and there may be a risk of unacceptable toxicity in combining treatments. For this reason, clinical trials of combination chemotherapy and monoclonal antibody therapy have tended to focus on relapsed and refractory neuroblastoma patients, who have fewer treatment options and worse outcomes. A clinical trial of irinotecan hydrochloride, temozolomide, and dinutuximab with or without eflornithine in treating patients with relapsed or refractory neuroblastoma recruitment has suspended patient recruitment because of a higher than expected incidence of hearing loss (https://clinicaltrials.gov/ct2/show/NCT03794349). Even if clinical trials on relapsed and refractory neuroblastoma patients proceed as planned, response rates would not necessarily predict response in newly diagnosed patients because previously treated tumors generally acquire additional mutations enabling them to become more resistant to further anticancer treatment than lesser treated or previously untreated tumors.

Individual anti-GD2 antibodies have different toxicity profiles, with different dosing and supportive medication requirements. It may therefore not be possible to substitute one anti-GD2 antibody for another without modification of therapy parameters.

There remains a need to improve induction chemotherapy regimens for newly diagnosed neuroblastoma patients.

The listing or discussion of an apparently prior-published document in this specification should not necessarily be taken as an acknowledgement that the document is part of the state of the art or is common general knowledge.

### SUMMARY OF THE INVENTION

A first aspect of the invention provides a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a cumulative dose of up to 500 mg/m², wherein a newly diagnosed neuroblastoma is treated in the patient.

A second aspect of the invention provides a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient in a dose of up to 100 mg/m² per cycle during one or more cycles of induction chemotherapy, wherein a newly diagnosed neuroblastoma is treated in the patient.

A third aspect of the invention provides a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a treatment density of up to 5 mg/m²/day, wherein a newly diagnosed neuroblastoma is treated in the patient.

### DESCRIPTION OF THE FIGURES

FIG. 1: Schema for combination induction chemotherapy (COJEC and GPOH) and combination dinutuximab beta immunotherapy. Abbreviations: COJEC=cisplatin, vincristine, carboplatin, etoposide, and cyclophosphamide; Cycle A=vincristine, carboplatin, etoposide; Cycle B=vincristine, cisplatin; Cycle C=vincristine, cyclophosphamide, etoposide; GPOH=German Pediatric Oncology and Hematology; HDC BuMel=high-dose chemotherapy busulfan and melphalan; Cycle N5=cisplatin, etoposide, vindesine; Cycle N6=vincristine, dacarbazine, ifosfamide, doxorubicin; PBSC=peripheral blood stem cells.

### DETAILED DESCRIPTION OF THE INVENTION

The present invention provides methods of treating a newly diagnosed neuroblastoma in a patient and compositions for use in the methods. Neuroblastomas are cancers that start in early nerve cells (called neuroblasts) of the sympathetic nervous system, and they can be found anywhere along this system. Most primary tumors (65%) occur within the abdomen with at least half of these arising in the adrenal medulla. Other common sites of disease include the neck, chest, and pelvis. Presenting signs and symptoms are highly variable and dependent on site of primary tumor as well as the presence or absence of metastatic disease and/or paraneoplastic syndromes. International Neuroblastoma Risk Group Staging System (INRGSS) is a clinical classification system that is determined prior to any treatment, including surgery, based on preoperative imaging. It classifies according to 2 stages of localized (Li and L2) and 2 stages of metastatic disease (M and MS) (Monclair T et al (2009) J. Clin. Oncol., 27:298-303, 2009). A new International Neuroblastoma Risk Group (INRG) classification system has been proposed in 2009 with 4 broad categories—very low risk, low risk, intermediate risk, and high risk—based on the assessment of the following prognostic factors: age at diagnosis (2 cutoffs, 12 and 18 months), INRG tumour stage (L1, L2, M, MS), histologic category, grade of tumour differentiation, DNA ploidy (hyperploidy/diploidy), MYCN oncogene status (amplified or not), aberrations at chromosome 11q (presence/absence) (Cohn S L et al, Journal of Clinical Oncology 2009 27:2, 289-297). This system uses combinations of the seven prognostic risk factors to define 16 pretreatment groups stratified by the prognostic markers within four categories, namely very low-, low-, intermediate- and or high-risk group, the categories based on the 5-year event-free survival (EFS) rates of the 16 pretreatment groups. The pretreatment groups (labeled A to R) and risk categories are summarized in Pinto N R et al, J Clin Oncol. 2015 Sep. 20; 33(27): 3008-3017, as shown in Table 1.

TABLE 1
Neuroblastoma pretreatment groups and risk categories

| INRG | Age | Histologic | Grade of Tumor | | 11q | | Pre-treatment |
|---|---|---|---|---|---|---|---|
| Stage | (months) | Category | Differentiation | MYCN | Aberration | Ploidy | Risk Group |
| L1/L2 | | GN maturing, | | | | | A (very low) |
| | | GNB | | | | | |
| | | intermixed | | | | | |
| L1 | | Any, except | | NA | | | B (very low) |
| | | GN maturing | | Amplified | | | K (high) |
| | | or GNB | | | | | |
| | | intermixed | | | | | |
| L2 | <18 | Any, except | | NA | No | | D (low) |
| | | GN maturing | | | Yes | | G (intermediate) |
| | | or GNB | | | | | |
| | | intermixed | | | | | |
| | ≥18 | GNB nodular | Differentiating | NA | No | | E (low) |
| | | neuroblastoma | | | Yes | | H (intermediate) |
| | | | Poorly | NA | | | H (intermediate) |
| | | | differentiated or | Amplified | | | |
| | | | undifferentiated | | | | N (high) |
| M | <18 | | | NA | | Hyperdiploid | F (low) |
| | <12 | | | NA | | Diploid | I (intermediate) |
| | 12 to <18 | | | NA | | Diploid | J (intermediate) |
| | <18 | | | Amplified | | | O (high) |
| | ≥18 | | | | | | P (high) |
| MS | <18 | | | NA | No | | C (very low) |
| | | | | | Yes | | Q (high) |
| | | | | Amplified | | | R (high) |

By newly diagnosed neuroblastoma, we mean a first diagnosis of neuroblastoma within a patient, for which the patient has yet to receive any neuroblastoma treatment. The newly diagnosed neuroblastoma may fall within any of the INRGSS disease classifications, or it may have been diagnosed by any other clinically accepted means. Typically, a newly diagnosed neuroblastoma patient will commence treatment within a few weeks from diagnosis, but we do not intend the term “newly diagnosed” to imply any limit on the interval between diagnosis and treatment.

The methods may suitably be used for treatment of high-risk neuroblastoma, according to the high-risk classification of the International Neuroblastoma Risk Group (INRG) classification system, as described in Pinto N R et al, supra. In particular, pretreatment groups K, N, O, P, Q and R are identified as belonging to the high-risk classification, and the methods may be used to treat patients in any of these groups, which may be defined as any patient with MYCN amplified neuroblastoma (other than stage Li according to INRGSS) or any patient older than 12 months of age at diagnosis with stage M disease. Suitable patients may be pretreatment group P (i.e. stage M, according to INRGSS, age 218 months), and typically <18 years.

The methods involve administering chimeric anti-GD2 antibody dinutuximab beta to the newly diagnosed neuroblastoma patient in combination with induction chemotherapy. dinutuximab beta consists of 2 light chains (220 amino acids) and 2 heavy chains (443 amino acids) and is of the IgG1 subclass. The monoclonal antibody incorporates human constant regions for the heavy chain IgG1 and the kappa light chain, along with the mouse variable regions targeted specifically against human GD2. The relative molecular mass of the intact antibody is approximately 150,000 daltons. The encoding nucleotide sequences and the amino acid sequences of chimeric anti-GD2 antibody dinutuximab beta are provided in U.S. Pat. No. 9,777,068 B2. In particular, the light-chain nucleotide sequence is provided as SEQ ID NO.1, the heavy-chain nucleotide sequence is provided as SEQ ID NO.2, the light-chain amino acid sequence as SEQ ID NO. 3, and the heavy-chain amino acid sequence as SEQ ID NO. 4. The first 60 nucleotides of SEQ ID NO. 1 or SEQ ID NO. 2 encode the signal peptide of the heavy or light chain respectively. The first 20 amino acids of SEQ ID NO. 3 or 4 are the signal peptide of the heavy or light chain respectively. The signal peptides are cleaved off during post-translational processing, and are not part of the final recombinant protein. Methods of manufacture and formulation of dinutuximab beta for clinical use are described in the European Public Assessment Report (EPAR) of the Committee for Medicinal Products for Human Use (CHMP) of the European Medicines Agency (EMA) for Dinutuximab beta Apeiron (EMA/263814/2017, 23 Mar. 2017).

A preparation comprising a dinutuximab beta may further comprise salts and WFI. In one embodiment, the preparation comprising dinutuximab beta may further comprise a buffer, e.g., phosphate-buffered saline, comprising said salts and WFI. A preparation comprising dinutuximab beta may further comprise stabilizing agents, preservatives and other carriers or excipients. The preparation comprising a dinutuximab beta may be freeze-dried and reconstituted for use. In one embodiment, the preparation comprising dinutuximab beta does not comprise preservatives and other excipients. The preparation comprising dinutuximab beta may be added to an infusion bag, e.g., an infusion bag containing 100 mL NaCl 0.9% and 5 mL human serum albumin 20%.

Chemotherapy for neuroblastoma typically involves multiple cycles of treatments with combinations of drugs. By induction therapy, we mean the first (frontline) or only course of chemotherapy administered to a newly diagnosed neuroblastoma patient. Induction chemotherapy is typically performed in conjunction with surgery. Induction chemotherapy may be administered before or after surgery, or surgery may be performed between treatment periods with induction chemotherapy drugs. Surgery may be performed more than once in conjunction with induction chemotherapy. Depending on risk classification, treatment may stop after induction chemotherapy and surgery. Induction therapy is typically used in the treatment of high-risk neuroblastoma, although patients classified as intermediate-risk may also receive chemotherapy and surgery (Pinto N R et al, 2015, supra). Multimodal standard treatment for newly diagnosed high risk neuroblastoma may involve induction chemotherapy, megatherapy, radiation, surgery, and consolidation therapy. For high-risk neuroblastomas, the current treatment can be divided into 3 distinct phases (Maris J M. N Engl J Med. 2010; 362(23):2202-2211. doi:10.1056/NEJMra0804577): (i) induction of remission with intensive chemotherapy. After a response to chemotherapy, resection of the primary tumour is usually attempted. (ii) consolidation of the remission with myeloablative chemotherapy which attempts to eradicate minimal residual disease using lethal doses of chemotherapy followed rapidly by rescue with autologous hematopoietic progenitor cells to repopulate the bone marrow. (iii) a maintenance phase used to treat potential minimal residual disease (MRD) following HSCT to reduce the risk of relapse (Matthay K K et al. [published correction appears in J Clin Oncol. 2014 Jun. 10; 32(17):1862-3]. J Clin Oncol. 2009; 27(7):1007-1013. doi:10.1200/JCO.2007.13.8925), e.g., with dinutuximab beta and isotretinoin, a molecule that induces terminal differentiation of neuroblastoma cell lines.

In general, during induction, patients receive 5-8 cycles of chemotherapy including platinum compounds, alkylating agents, and topoisomerase inhibitors (Smith, V., & Foster, J. (2018). High-Risk Neuroblastoma Treatment Review. Children (Basel, Switzerland), 5(9), 114. https://doi.org/10.3390/children5090114). Other commonly used agents are antimicrotubule agents and anthracyclines. Suitable platinum compounds may include cisplatin, carboplatin and/or oxaliplatin. Suitable alkylating agents may include cyclophosphamide, dacarbazine, mechlorethamine, chlorambucil, temozolamide, melphalan, and/or ifosfamide. Suitable topoisomerase agents may inhibit topoisomerase I or topoisomerase II. Suitable topoisomerase I inhibitors may include irinotecan, topotecan, and/or camptothecin. Suitable topoisomerase II inhibitors may include etoposide, doxorubicin, epirubicin, daunorubicin and/or mitoxantrone. Anthracyclines are a class of potent and widely used cytotoxic drugs, derived from antibiotics that inhibit DNA and RNA synthesis by intercalating between base pairs of the DNA/RNA strand. They create iron-mediated free oxygen radicals, damaging the DNA and cell membranes, and inhibit topoisomerase I. Suitable anthracyclines may include doxorubicin, epirubicin, daunorubicin and/or mitoxantrone. Suitable antimicrotubule agents include vinca alkaloids, which are made from the periwinkle plant (Catharanthus rosea), and taxanes, which are derived from the bark of the Pacific Yew tree (taxus). Suitable vinca alkaloids are vincristine, vinblastine, vinorelbine, and/or vindesine. Suitable taxanes include paclitaxel and/or docetaxel. By induction chemotherapy we include any chemotherapy including at least one agent selected from at least three of the four classes, namely platinum compounds, alkylating agents, topoisomerase inhibitors and microtubule agents. Suitably, the induction chemotherapy comprises between 1 and 10 cycles, such as between 5 and 8 cycles. Suitable numbers of cycles may be 1, 2, 3, 4, 5, 6, 7, 8, 9 or 10 cycles.

Induction chemotherapy regimens which have been investigated are summarized in the table below.

Of these, the 3 most effective induction chemotherapy regimens have become standard of care in different geographic regions.

The rapid COJEC regimen is the preferred European induction regimen from the SIOPEN group, described in Ladenstein R et al. J Clin Oncol. 2010; 28(21):3516-3524. Rapid COJEC showed an improvement in the Phase 3 HRNBL1 Study against the modified MSKCC/COG N7 (https://clinicaltrials.gov/ct2/show/NCT01704716). In the rapid COJEC induction schedule, higher single doses of selected drugs than standard induction schedules are administered over a substantially shorter treatment period, with shorter intervals between cycles (Cochrane Database Syst Rev. 2015 May 19(5):CD010774. doi: 10.1002/14651858.CD010774.pub2).

TABLE 2
Induction chemotherapy regimens

| Regimen | Schedule | Survival | Evidence |
|---|---|---|---|
| COG A3973 | alternating | 5-year EFS 38% | Single arm |
| Kreissman | CAV + P/E × 6 | 5-year OS 50% | prospective trial |
| Lancet Oncol | | | |
| (n = 486) | | | |
| NB97/GPOH | alternating | 3-year EFS 39% | Single arm |
| Berthold | N5 + N6 × 6 | 3-year OS 58% | prospective trial |
| Lancet Oncol | | | |
| ENGS5 | OPEC/OJEC vs | 5-year EFS | Randomized trial |
| Pearson | COJEC | 18.2% vs 30.2% | |
| Lancet Oncol | | (p = 0.022) | |
| (n = 262) | | | |
| CCG3971 | 5 cycles of CDDP, | 3-year EFS 30% | Single arm |
| Matthay | DOX, VP, CPM | 2-year OS 45% | prospective trial |
| NEJM | | | |
| ANBL0532/COG | T/C × 2, P/E, CAV, | 3-year EFS 51% | Single arm |
| Park | P/E, CAV | 2-year OS 68% | prospective trial |
| JCO | | | |
| HR-NBL1/SIOPEN | Rapid COJEC vs | 3-year EFS | Randomized trial |
| Ladenstein | modified N7 (CAV + | 39% vs 39% | |
| Preliminary results | P/E × 5) | (p = 0.805) | |
| (n = 607) | | | |
| N82004-HR/GPOH | N5/N6 × 6 vs | NS/N6 arm: | Randomized trial |
| Berthold | N8/N5/N6 × 8 | 3-year EFS 36% | |
| Preliminary results | | | |

The preferred German regimen since 1997 is GPOH (Gesellschaft für Pädiatrische Onkologie und Hämatologie) NB2004 N5/N6, which involves 6 alternating chemotherapy cycles of N5 (cisplatin, etoposide, and vindesine) and N6 (ifosfamide, vincristine, dacarbazine, and doxorubicin) (Simon T et al. Klin Padiatr. 2017; 229(3):147-67.6; and Berthold F, Lancet Oncol. 2005; 6(9):649-58). Complete responses (CR) or very good partial responses were achieved in 55% of patients.

The preferred US induction regimen from the Children's Oncology Group (COG) (ANBL0532 program) is described in Park J R et al, J Clin Oncol 2011; 29(33):4351-57; and https://clinicaltrials.gov/ct2/show/NCT00567567. ANBL/COG involves 2 cycles of topotecan and cyclophosphamide (400 mg/m²/d) for 5 days followed by 4 cycles of multiagent chemotherapy (Memorial Sloan-Kettering Cancer Center [MSKCC] regimen comprising alternating cisplatin/etoposide and cyclophosphamide plus doxorubicin/vincristine).

A “treatment period” with a specific preparation or treatment as used herein means the period of time in which said specific preparation or treatment is administered to the patient. For example, if a chemotherapy drug is administered for 8 consecutive days, followed by 2 days of no administration of the chemotherapy drug, then the treatment period with the chemotherapy drug is 8 days.

The term “treatment cycle” as used herein means a course of one or more treatments or treatment periods that is repeated on a regular schedule and may encompass a period of rest. For example, a treatment given for 8 days followed by 2 days of rest is 1 treatment cycle. The treatment cycle may be repeated, either identically or in an amended form, e.g., with a different dose or schedule, or with different additional treatments. A “treatment interval” is the interval between starting and completing a treatment cycle.

The “overall treatment time” means the time period comprising all treatment cycles. As described above, treatment cycles may comprise time periods of no treatment (intervals in which no treatment is administered to the patient, i.e., no chemotherapy, no antibody, no other drug). Thus, as used herein, the overall treatment time may also comprise said intervals of no treatment within treatment cycles. For example, if the patient receives 8 treatment cycles of 10 days, then the overall treatment time is 80 days. The overall treatment time may comprise at least 1, or 2 or more cycles, or up to 12 cycles. In one embodiment, the overall treatment time comprises 3, 4, 5, 6, 7, 8, 9, or 10 cycles.

By administering chimeric anti-GD2 antibody dinutuximab beta in combination with induction chemotherapy, we mean that the anti-GD2 antibody is administered during the overall treatment time of the induction chemotherapy. Within a given treatment cycle, the anti-GD2 antibody and the chemotherapy drug(s) may be administered simultaneously, sequentially, or separately. As used herein “simultaneously” means that the drugs are to be taken together on at least one treatment day and may or may not be formulated as a single composition. Simultaneously” also encompasses a partial overlap in treatment days upon which the drugs are administered. For example, the chemotherapy drug(s) may be administered for one or more consecutive days, and then both the chemotherapy drug(s) and the anti-GD2 antibody may be administered on subsequent consecutive days. “Sequentially” means that the drugs are administered on consecutive treatment days, but not on the same treatment day. For example, the chemotherapy drug(s) may be administered for one or more consecutive days, and the anti-GD2 antibody may be administered for the immediately following one or more consecutive days. As used herein, “separate” administration means that the anti-GD2 antibody and the chemotherapy drug(s) are administered as part of the same overall dosing regimen, but they are not administered on the same day. For example, the chemotherapy drug(s) may be administered for one or more consecutive days, then there may be one or more days during which neither the chemotherapy drug(s) nor the anti-GD2 antibody are administered, and then on one or more subsequent days, the anti-GD2 antibody may be administered. Typically, the anti-GD2 antibody is administered simultaneously with the chemotherapy drug(s), more typically the treatment periods of the anti-GD2 antibody and the chemotherapy drug(s) are partially overlapping.

According to the first aspect of the invention, the chimeric anti-GD2 antibody dinutuximab beta is administered to the patient during the induction chemotherapy at a cumulative dose of up to 500 mg/m². By “cumulative dose”, we mean the total dose that is administered during the overall treatment time. The units of dose are expressed in mg/m², where the area (in m²) refers to the patient's body surface area (BSA). For example, if a patient has a body surface area of 0.7 m², then a cumulative dose of 500 mg/m² would be 350 mg.

Suitably, the chimeric anti-GD2 antibody dinutuximab beta is administered to the patient during the induction chemotherapy at a cumulative dose of at least 100 mg/m², such as from 100 to 200 mg/m², from 200 to 300 mg/m² from 300 to 400 mg/m², or from 400 mg/m² to 500 mg/m². Any intermediate range is also envisaged, such as from 100 to 400 mg/m², or from 200 to 400 mg/m².

According to the second aspect of the invention, the chimeric anti-GD2 antibody dinutuximab beta is administered to the patient in a dose of up to 100 mg/m² per cycle during one or more cycles of induction chemotherapy. Dinutuximab beta is not necessarily administered during all cycles of the induction chemotherapy. It may be administered during all but the first 2 cycles, or all but the first cycle, or all cycles of induction chemotherapy. Omitting the dinutuximab beta from the first cycle or first 2 cycles may improve the tolerability of the treatment regimen, as patients may start to adapt to the toxicity of the chemotherapy drug(s) after 1 or 2 cycles.

In one embodiment, the chimeric anti-GD2 antibody dinutuximab beta is administered to the patient during the induction chemotherapy in a dose per cycle that is equal for all cycles during which the chimeric anti-GD2 antibody dinutuximab beta is administered. For example, dinutuximab beta may be administered in a dose per cycle that is at least 10 mg/m² per cycle, such as from 10 to 30 mg/m² per cycle, from 20 to 40 mg/m² per cycle, from 30 to 50 mg/m² per cycle, from 40 to 60 mg/m² per cycle, from 50 to 70 mg/m² per cycle, or from 60 to 80 mg/m² per cycle. Suitable doses per cycle may be 20 mg/m², 30 mg/m², 40 mg/m², 50 mg/m², 60 mg/m², or 70 mg/m².

In another embodiment, dinutuximab beta may be administered at a dose per cycle that varies by up to 20 mg/m² such as up to 10 mg/m² between different cycles during which the chimeric anti-GD2 antibody dinutuximab beta is administered. The choice to vary the dose of dinutuximab beta between cycles may be based on the toxicity profiles of different cycles of a given induction chemotherapy. For example, dinutuximab beta may be administered in a dose per cycle of at least 10 mg/m² per cycle for all cycles, such as from 10 to 30 mg/m² per cycle for one or more cycles and from 20 to 40 mg/m² per cycle for the other cycles during which the chimeric anti-GD2 antibody dinutuximab beta is administered; or from 20 to 40 mg/m² per cycle for one or more cycles and from 30 to 50 mg/m² per cycle for the other cycles during which the chimeric anti-GD2 antibody dinutuximab beta is administered; or from 20 to 40 mg/m² per cycle for one or more cycles and from 40 to 60 mg/m² per cycle for the other cycles during which the chimeric anti-GD2 antibody dinutuximab beta is administered; or from 40 to 60 mg/m² per cycle for one or more cycles and from 60 to 80 mg/m² per cycle for the other cycles during which the chimeric anti-GD2 antibody dinutuximab beta is administered. For any of the above regimens, the mid-point of the given range may suitably be administered. For example, for the range of 20 to 40 mg/m² per cycle, a suitable dose of dinutuximab beta would be 30 mg/m².

Any of the above features of dose per cycle as defined in relation to the second aspect of the invention may be applied to the first aspect of the invention. In other words, a cumulative dose of up to 500 mg/m², such as at least 100 mg/m², such as from 100 to 200 mg/m², from 200 to 300 mg/m², from 300 to 400 mg/m², or from 400 to 500 mg/m² of dinutuximab beta may be provided in doses per cycle according to the embodiments described above. Moreover, the features of cumulative dose as defined in relation to the first aspect of the invention may be applied to the second aspect of the invention. In other words, when the dinutuximab beta is administered at doses of up to 100 mg/m² per cycle during one or more cycles of induction chemotherapy, such as described in any of the embodiments above, the cumulative dose may be up to 500 mg/m², and embodiments described above.

According to the third aspect of the invention, the chimeric anti-GD2 antibody dinutuximab beta is administered to the patient during the induction chemotherapy at a treatment density of up to 5 mg/m²/day. By “treatment density” we mean the cumulative dose divided by the total duration of combined induction therapy and dinutuximab beta treatment cycles. Where dinutuximab beta is omitted from the first one more treatment cycles, the “overall treatment time for dinutuximab beta” is less than the overall treatment time. The “overall treatment time for dinutuximab beta” is measured from the beginning of the first to the end of the last treatment cycle during which the dinutuximab beta is administered. For example, if the induction chemotherapy comprises 8 treatment cycles of 10 days each, and dinutuximab beta is first administered during the third cycle, and last administered during the eighth cycle, then the overall treatment time is 80 days, and the overall treatment time for dinutuximab beta is 60 days. Thus “treatment density” may also be defined as the cumulative dose divided by the “overall treatment time for dinutuximab beta”. For example, if a cumulative dose of dinutuximab beta of 180 mg/m² is administered during six 10-day cycles, then the treatment density is 3 mg/m². Suitable treatment densities may be at least 1 mg/m²/day, such as from 1 to 2 mg/m²/day, from 2 to 3 mg/m²/day or from 3 to 4 mg/m²/day. Any intermediate range is also envisaged, such as a treatment density of from 1 to 4 mg/m²/day, or from 2 to 4 mg/m²/day.

Any of the above features of treatment as defined in relation to the third aspect of the invention may be applied to the first aspect of the invention. In other words, a cumulative dose of up to 500 mg/m², such as at least 100 mg/m², such as from 100 to 200 mg/m², from 200 to 300 mg/m², from 300 to 400 mg/m², or from 400 to 500 mg/m² of dinutuximab beta may be provided at a treatment density according to the embodiments described above. Any of the above features of treatment density as defined in relation to the third aspect of the invention may be applied to the second aspect of the invention. In other words, when the dinutuximab beta is administered at doses of up to 100 mg/m² per cycle during one or more cycles of induction chemotherapy, such as described in any of the embodiments above, the treatment density may be up to 5 mg/m²/day, and embodiments described above. Moreover, the features of cumulative dose as defined in relation to the first aspect of the invention may be applied to the third aspect of the invention. In other words, when the dinutuximab beta is administered at a treatment density of up to 5 mg/m²/day, such as described in any of the embodiments above, the cumulative dose may be up to 500 mg/m², and embodiments described above. Finally, the features of defined in relation to the second aspect of the invention may be applied to the third aspect of the invention. In other words, when the dinutuximab beta is administered at a treatment density of up to 5 mg/m²/day, such as described in any of the embodiments above, the dinutuximab beta may be administered in doses per cycle according to the embodiments described above.

In all aspects of the invention, the dinutuximab beta dose may be selected such that the incidence of dose-limiting toxicity (DLT) falls within a target range. The incidence of DLT may be expressed either as the percentage of patients who experience DLT during combination chemotherapy and dinutuximab beta therapy, or as a rate where 0 is no patients and 1 is all patients. The incidence of DLT may be determined in a clinical trial and may be expected to also apply to subsequent patients who receive the therapy. A target DLT rate may be set for a clinical trial and a permitted range of DLT rates. A suitable target DLT rate is ≤33% of patients, and a permitted range of DLT rates is <39.5%, such as 26.9%<x<39.5%. A DLT is defined as a dinutuximab beta-related adverse event occurring during the DLT assessment period in a clinical trial that leads to treatment discontinuation or which meets certain criteria of Grade ≥3 or 4 toxicity using National Cancer Institute (NCI) Common Terminology Criteria for Adverse Events (CTCAE) version 5.0. Suitable criteria are described in the Example.

In any of the aspects of the invention, the chimeric anti-GD2 antibody dinutuximab beta may be administered to the patient as a continuous intravenous infusion over 24 hours per day, at a daily dose of 10 mg/m². Alternatively, it may be administered over fewer than 24 hours per day, such as over between 8 and 24 hours, or over 8 hours, or over 4 hours. It may be administered at a daily dose of less than 10 mg/m², such as 5 mg/m². The antibody should be prepared under sterile conditions. The appropriate volume of dinutuximab beta should be withdrawn from the vials. It is recommended that the antibody solution is filtered (0.2 to 1.2 μm) before injection into the patient either by using an in-line filter during infusion or by filtering the solution with a particle filter (e.g., filter Nr. MF1830, Impromediform, Germany). The volume of the antibody is typically added to an infusion bag containing 100 mL NaCl 0.9% and 5 mL human serum albumin 20%. In any of the aspects of the invention, the chimeric anti-GD2 antibody dinutuximab beta may be administered to the patient on consecutive days of a cycle until the entire dose per cycle of the chimeric anti-GD2 antibody dinutuximab beta has been administered.

Pain is an anticipated side effect of dinutuximab beta administration, which has been managed by standard pain prophylaxis, including intravenous morphine. In some embodiments, morphine is administered only for some but not all days on which the antibody is administered, e.g., only on the first 1, 2, 3, 4, 5, 6, or 7 days of continuous antibody infusion. Further information on management of pain using morphine and other drugs is described in U.S. Pat. No. 9,777,068B2.

Other drugs may be included in induction chemotherapy cycles, such as isotretinoin (13-cis-retinoic acid), suitably after the completion of the dinutuximab beta infusion. Granulocyte-colony stimulating factor (G-CSF) may be included as supportive therapy for chemotherapy, such as described in relation to the exemplified COJEC and GPOH protocols.

The first, second, and third aspects of the invention have been particularly exemplified in relation to an induction chemotherapy regimen comprising consecutive chemotherapy cycles of A (vincristine, carboplatin and etoposide), B (vincristine and cisplatin), C (vincristine, cyclophosphamide and etoposide), B, A, B, C, and B, wherein the cycles are of at least 10 days. A suitable regimen is rapid COJEC, also referred to herein simply as COJEC (Ladenstein R et al. J Clin Oncol. 2010; 28(21):3516-3524). Typically, the chemotherapy cycles in rapid COJEC are of 10 day duration, but the cycles may be up to 14 days duration, according to Smith and Foster, 2018, supra. Cycle durations of 10, 11, 12, 13 and 14 days are encompassed. According to different embodiments, the chimeric anti-GD2 antibody dinutuximab beta is administered to the patient at (i) a cumulative dose of from 120 to 160 mg/m² and/or a treatment density of from 2.00 to 2.67 mg/m²/day; or (ii) at a cumulative dose of from 160 to 200 mg/m² and/or a treatment density of from 2.67 to 3.33 mg/m²/day; or (iii) at a cumulative dose of from 190 to 230 mg/m² and/or a treatment density of from 3.17 to 3.83 mg/m²/day; or (iv) at a cumulative dose of from 220 to 260 mg/m² and/or a treatment density of from 3.67 to 4.30 mg/m²/day. Within option (i) above, a suitable cumulative dose of 140 mg/m² may be provided in 4 cycles at 20 mg/m² per cycle and 2 cycles at 30 mg/m² per cycle (amounting to a treatment density of 2.33 mg/m²/day). Within option (ii) above, a suitable cumulative dose of 180 mg/m² may be provided in 6 cycles at 30 mg/m² per cycle (amounting to a treatment density of 3.00 mg/m²/day). Within option (iii) above, a suitable cumulative dose of 210 mg/m² may be provided in 3 cycles at 30 mg/m² per cycle and 3 cycles at 40 mg/m² per cycle (amounting to a treatment density of 3.50 mg/m²/day). Within option (iv) above, a suitable cumulative dose of 240 mg/m² may be provided in 6 cycles at 40 mg/m² per cycle (amounting to a treatment density of 4.00 mg/m²/day). In each of these options, the dinutuximab beta is suitably administered during all but the first 2 cycles of the induction chemotherapy.

The first, second, and third aspects of the invention have been particularly exemplified in relation to an induction chemotherapy regimen comprising 6 alternating chemotherapy cycles of N5 (cisplatin, etoposide, and vindesine) and N6 (ifosfamide, vincristine, dacarbazine, and doxorubicin), wherein the cycles are of at least 21 days. A suitable regimen is GPOH (Simon T et al. Klin Padiatr. 2017; 229(3):147-67.6; Berthold F, Lancet Oncol. 2005; 6(9):649-58). Typically, the chemotherapy cycles in GPOH are of 21 days duration, but the cycles may be longer. Cycle durations of 21, 22, 23, 24, 25, 26, 27 or 28 days are encompassed. According to different embodiments, the chimeric anti-GD2 antibody dinutuximab beta is administered to the patient at (i) a cumulative dose of from 160 to 220 mg/m² and/or a treatment density of from 1.52 to 2.10 mg/m²/day; or (ii) at a cumulative dose of from 220 to 280 mg/m² and/or a treatment density of from 2.10 to 2.67 mg/m²/day; or (iii) at a cumulative dose of from 280 to 340 mg/m² and/or a treatment density of from 2.67 to 3.24 mg/m²/day; or (iv) at a cumulative dose of from 320 to 380 mg/m² and/or a treatment density of from 3.05 to 3.62 mg/m²/day. Within option (i) above, a suitable cumulative dose of 190 mg/m² may be provided in 2 cycles at 30 mg/m² per cycle and 3 cycles at 50 mg/m² per cycle (amounting to a treatment density of 1.81 mg/m²/day). Within option (ii) above, a suitable cumulative dose of 250 mg/m² may be provided in 5 cycles at 50 mg/m² per cycle (amounting to a treatment density of 2.38 mg/m²/day). Within option (iii) above, a suitable cumulative dose of 310 mg/m² may be provided in 2 cycles at 50 mg/m² per cycle and 3 cycles at 70 mg/m² per cycle (amounting to a treatment density of 2.95 mg/m²/day). Within option (iv) above, a suitable cumulative dose of 350 mg/m² may be provided in 5 cycles at 70 mg/m² per cycle (amounting to a treatment density of 3.33 mg/m²/day). In each of these options, the dinutuximab beta is suitably administered during all but the first cycle of the induction chemotherapy.

In certain embodiments, the therapeutic effect of the combination induction chemotherapy and dinutuximab beta therapy may be defined as stable disease (i.e., no further increase in lesions, tumor tissue and/or size), partial response (i.e., reduction in lesions, tumor tissue and/or size), and/or complete response (i.e., complete remission of all lesions and tumor tissue). In some embodiments, the therapeutic effect of dinutuximab beta administration may be an increase in immune response to the tumor, as determined, for example, by an increase in immune system biomarkers (e.g., blood parameters, such as lymphocyte counts and/or NK cell numbers; and/or cytokines). In some embodiments, the therapeutic effect may be a reduction in tumor markers (e.g., catecholamines). In some embodiments, the therapeutic effect may be determined by methods such as metaiodobenzylguanidine scintigraphy (mIBG), magnetic resonance imaging (MRI), or X-ray computed tomography (CT), and/or bone marrow histology (assessed by aspirate or trephine biopsy).

Suitably, in any of the aspects of the invention, the administration of the chimeric anti-GD2 antibody dinutuximab beta improves one or more clinical parameters compared to the induction chemotherapy administered without the chimeric anti-GD2 antibody dinutuximab beta. International neuroblastoma response criteria are described in Park J R et al J Clin Oncol. 2017; 35(22):2580. Suitable clinical parameters are selected from overall response rate (ORR), complete response (CR) rate, partial response (PR) rate, primary tumour volume reduction, Curie score, event-free survival (EFS), and overall survival (OS), overall response during and after induction (primary tumor, metastases), and metastatic CR and PR rates. EFS and OS may be determined at 3 or 5 years. ORR includes CR, and PR. Clinical improvement may also be characterized by changes in immune parameters during induction therapy for patients also treated with dinutuximab beta. Suitable immune parameters are Immunophenotype, complement-dependent cytotoxicity (CDC) and ADCC.

Complete Response (CR) may be further defined as follows:
Complete disappearance of all measurable and evaluable disease,
no new lesions,
no disease-related symptoms, and/or
no evidence of evaluable disease, including, e.g., normalization of markers and/or other abnormal laboratory values.

In some embodiments, all measurable, evaluable, and non-evaluable lesions and sites must be assessed using the same technique as baseline.

Partial Response (PR) may be further defined as follows:
Applies only to patients with at least 1 measurable lesion.
Greater than or equal to 50% decrease under baseline in the sum of products of perpendicular diameters of all measurable lesions.
No progression of evaluable disease.
No new lesions.

A fourth aspect of the invention provides a chimeric anti-GD2 antibody dinutuximab beta for use in the method of the first aspect of the invention. A fifth aspect of the invention provides a chimeric anti-GD2 antibody dinutuximab beta for use in the method of the second aspect of the invention. A sixth aspect of the invention provides a chimeric anti-GD2 antibody dinutuximab beta for use in the method of the third aspect of the invention. The preferred features of the first, second, and third aspect of the invention are equally applicable to the fourth, fifth, and sixth aspects, respectively.

Preferences and options for a given aspect, feature or parameter of the invention should, unless the context dictates otherwise, be regarded as having been disclosed in combination with any and all preferences and options for all other aspects, features and parameters of the invention.

All documents are incorporated by reference in their entirety.

Embodiments of the invention will now be described in the following numbered paragraphs:
1. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a cumulative dose of up to 500 mg/m², wherein a newly diagnosed neuroblastoma is treated in the patient.
2. The method of Paragraph 1, wherein the chimeric anti-GD2 antibody dinutuximab beta is administered to the patient during the induction chemotherapy at a cumulative dose of at least 100 mg/m², such as from 100 to 200 mg/m², from 200 to 300 mg/m², from 300 to 400 mg/m², or from 400 to 500 mg/m².
3. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient in a dose of up to 100 mg/m² per cycle during one or more cycles of induction chemotherapy, wherein a newly diagnosed neuroblastoma is treated in the patient.
4. The method of Paragraph 3, wherein the chimeric anti-GD2 antibody dinutuximab beta is administered to the patient during all but the first 2 cycles, or all but the first cycle, or all cycles of the induction chemotherapy.
5. The method of Paragraph 3 or 4, wherein the chimeric anti-GD2 antibody dinutuximab beta is administered to the patient during the induction chemotherapy in a dose per cycle that is equal for all cycles during which the chimeric anti-GD2 antibody dinutuximab beta is administered.
6. The method of Paragraph 6, wherein the chimeric anti-GD2 antibody dinutuximab beta is administered to the patient during the induction chemotherapy in a dose per cycle that is at least 10 mg/m² per cycle, such as from 10 to 30 mg/m² per cycle, from 20 to 40 mg/m² per cycle, from 30 to 50 mg/m² per cycle, from 40 to 60 mg/m² per cycle, from 50 to 70 mg/m² per cycle or from 60 to 80 mg/m² per cycle.
7. The method of Paragraph 3 or 4, wherein the chimeric anti-GD2 antibody dinutuximab beta is administered to the patient during the induction chemotherapy in a dose per cycle that varies by up to 20 mg/m² such as up to 10 mg/m² between different cycles during which the chimeric anti-GD2 antibody dinutuximab beta is administered.
8. The method of Paragraph 7, wherein the chimeric anti-GD2 antibody dinutuximab beta is administered to the patient during the induction chemotherapy in a dose per cycle of at least 10 mg/m² per cycle for all cycles, such as from 10 to 30 mg/m² per cycle for one or more cycles and from 20 to 40 mg/m² per cycle for the other cycles during which the chimeric anti-GD2 antibody dinutuximab beta is administered; or from 20 to 40 mg/m² per cycle for one or more cycles and from 30 to 50 mg/m² per cycle for the other cycles during which the chimeric anti-GD2 antibody dinutuximab beta is administered; or from 20 to 40 mg/m² per cycle for one or more cycles and from 40 to 60 mg/m² per cycle for the other cycles during which the chimeric anti-GD2 antibody dinutuximab beta is administered; or from 40 to 60 mg/m² per cycle for one or more cycles and from 60 to 80 mg/m² per cycle for the other cycles during which the chimeric anti-GD2 antibody dinutuximab beta is administered.
9. The method of any one of Paragraphs 3 to 8, wherein the chimeric anti-GD2 antibody dinutuximab beta is administered to the patient during the induction chemotherapy in a dose per cycle of 20 mg/m², 30 mg/m², 40 mg/m², 50 mg/m², 60 mg/m², or 70 mg/m² during one or more cycles of induction chemotherapy.
10. The method of Paragraph 1 or 2, wherein the chimeric anti-GD2 antibody dinutuximab beta is administered to the patient during the induction chemotherapy in a dosing regimen as defined in any one of Paragraphs 3 to 9.
11. The method of any one of Paragraphs 3 to 9, wherein the chimeric anti-GD2 antibody dinutuximab beta is administered to the patient during the induction chemotherapy at a cumulative dose as defined in Paragraph 1 or 2.
12. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a treatment density of up to 5 mg/m²/day, wherein a newly diagnosed neuroblastoma is treated in the patient.
13. The method of Paragraph 12, wherein the chimeric anti-GD2 antibody dinutuximab beta is administered to the patient during the induction chemotherapy at a treatment density of at least 1 mg/m²/day, such as from 1 to 2 mg/m²/day, from 2 to 3 mg/m²/day, or from 3 to 4 mg/m²/day.
14. The method of Paragraph 1, 2, or 10, wherein the chimeric anti-GD2 antibody dinutuximab beta is administered to the patient during the induction chemotherapy at a treatment density as defined in Paragraph 12 or 13.
15. The method of any one of Paragraphs 3 to 9 or 11, wherein the chimeric anti-GD2 antibody dinutuximab beta is administered to the patient during the induction chemotherapy at a treatment density as defined in Paragraph 12 or 13.
16. The method of Paragraph 12 or 13, wherein the chimeric anti-GD2 antibody dinutuximab beta is administered to the patient during the induction chemotherapy at a cumulative dose as defined in Paragraph 1 or 2.
17. The method of Paragraph 12 or 13, wherein the chimeric anti-GD2 antibody dinutuximab beta is administered to the patient during the induction chemotherapy in a dosing regimen as defined in any one of Paragraphs 3 to 9.
18. The method of any one of the preceding paragraphs wherein the chimeric anti-GD2 antibody dinutuximab beta is administered to the patient as a continuous intravenous infusion over 24 hours per day, in a daily dose of 10 mg/m².
19. The method of any one of the preceding paragraphs wherein the chimeric anti-GD2 antibody dinutuximab beta is administered to the patient on consecutive days of a cycle until all of the dose per cycle of the chimeric anti-GD2 antibody dinutuximab beta has been administered.
20. The method of any one of the preceding paragraphs wherein the induction chemotherapy comprises 6 alternating chemotherapy cycles of N5 (cisplatin, etoposide, and vindesine) and N6 (ifosfamide, vincristine, dacarbazine, and doxorubicin), wherein the cycles are of at least 21 days.
21. The method of Paragraph 20 wherein the chimeric anti-GD2 antibody dinutuximab beta is administered to the patient at a cumulative dose of from 160 to 220 mg/m² and/or a treatment density of from 1.52 to 2.10 mg/m²/day; or at a cumulative dose of from 220 to 280 mg/m² and/or a treatment density of from 2.10 to 2.67 mg/m²/day; or at a cumulative dose of from 280 to 340 mg/m² and/or a treatment density of from 2.67 to 3.24 mg/m²/day; or at a cumulative dose of from 320 to 380 mg/m² and/or a treatment density of from 3.05 to 3.62 mg/m²/day.
22. The method of any one of Paragraphs 1 to 19 wherein the induction chemotherapy comprises consecutive chemotherapy cycles of A (vincristine, carboplatin and etoposide), B (vincristine and cisplatin), C (vincristine, cyclophosphamide and etoposide), B, A, B, C and B, wherein the cycles are of at least 10 days.
23. The method of Paragraph 22 wherein the chimeric anti-GD2 antibody dinutuximab beta is administered to the patient at a cumulative dose of from 120 to 160 mg/m² and/or a treatment density of from 2.00 to 2.67 mg/m²/day; or at a cumulative dose of from 160 to 200 mg/m² and/or a treatment density of from 2.67 to 3.33 mg/m²/day; or at a cumulative dose of from 190 to 230 mg/m² and/or a treatment density of from 3.17 to 3.83 mg/m²/day; or at a cumulative dose of from 220 to 260 mg/m² and/or a treatment density of from 3.67 to 4.30 mg/m²/day.
24. The method of any preceding paragraph wherein the administration of the chimeric anti-GD2 antibody dinutuximab beta improves one or more clinical parameters compared to the induction chemotherapy administered without the chimeric anti-GD2 antibody dinutuximab beta.
25. The method of Paragraph 24 wherein the one of more clinical parameters are selected from complete response rate, partial response rate, primary tumour volume reduction, Curie score, event free survival and overall survival.
26. A chimeric anti-GD2 antibody dinutuximab beta for use in a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a cumulative dose of up to 500 mg/m².
27. A chimeric anti-GD2 antibody dinutuximab beta for use in a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient in a dose of up to 100 mg/m² per cycle during one or more cycles of induction chemotherapy.
28. A chimeric anti-GD2 antibody dinutuximab beta for use in a method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a treatment density of up to 5 mg/m²/day, wherein a newly diagnosed neuroblastoma is treated in the patient.
29. A chimeric anti-GD2 antibody dinutuximab beta for use of Paragraph 26, 27, or 28 wherein the method of treating a newly diagnosed neuroblastoma in a patient has any of the additional features of Paragraphs 1 to 25.

The present invention will be further illustrated in the following examples, without any limitation thereto.

### EXAMPLES

### Example 1: Phase 1 Study Combining Dinutuximab Beta with Induction Chemotherapy Regimens in Patients with Newly Diagnosed High-Risk Neuroblastoma

Rationale: This study is designed to identify safe and effective infusion durations for and cumulative dose levels of dinutuximab beta when combined with different induction chemotherapy regimens (German Pediatric Oncology and Hematology [GPOH] or cisplatin, vincristine, carboplatin, etoposide, and cyclophosphamide [COJEC]) for the treatment of newly diagnosed patients with high-risk neuroblastoma without substantially exceeding the treatment density previously established for dinutuximab beta monotherapy. The information will be used in the planning of future studies of an established induction chemotherapy regimen randomized to GPOH or COJEC with or without dinutuximab beta.

TABLE 3
Objectives and Endpoints

| Objectives | Endpoints |
|---|---|
| Primary | |
| To assess the safety and tolerability and | Incidences of DLTs associated with the |
| identify the MTDs of dinutuximab beta | combination of dinutuximab beta with |
| when combined with different induction | GPOH or COJEC induction chemotherapy |
| chemotherapy regimens (GPOH or | regimens. |
| COJEC) for the treatment of newly | |
| diagnosed high-risk neuroblastoma | |
| patients ≥18 months of age as defined | |
| by Stage M, according to the INRGSS. | |
| Secondary | |
| To characterize the toxicity of induction | Type, incidence, severity, seriousness |
| chemotherapy when combined with | and relationship to study medications for |
| dinutuximab beta. | AEs, including laboratory abnormalities |
| | and SAES. |
| | Cumulative incidence of treatment- |
| | related mortality and of disease-related |
| | mortality. |
| To determine the overall response | Overall response during and after |
| (primary tumor, metastases) at the end | induction (primary tumor, metastases). |
| of induction chemotherapy with | |
| dinutuximab beta. | |
| To determine the metastatic response | mCR after 2 cycles GPOH and after |
| rate in response to chemotherapy plus | 4 cycles COJEC. |
| dinutuximab beta. | mCR after induction treatment. |
| | mPR according to eligibility criteria to |
| | proceed to consolidation by HDC/ASCT: |
| | mPR for bone disease: MIBG |
| | uptake (or FDG-PET uptake for |
| | MIBG-nonavid tumors) |
| | completely resolved or SIOPEN |
| | score ≤3 and at least 50% |
| | reduction in MIBG score (or ≤3 |
| | bone lesions and at least 50% |
| | reduction in number of FDG-PET- |
| | avid bone lesions for |
| | MIBG-nonavid tumors). |
| | mPR for bone marrow disease: |
| | CR and/or MD according to INRC. |
| | mPR for other metastatic sites: |
| | complete response after |
| | induction chemotherapy +/− |
| | surgery. |
| Exploratory | |
| To determine the impact of GPOH or | 3-year and 5-year OS, calculated from |
| COJEC with dinutuximab beta induction | date of enrollment to the date of last |
| therapy on patient OS. | follow-up or death from any cause. |
| To determine the impact of GPOH or | 3-year and 5-year EFS from the date of |
| COJEC with dinutuximab beta induction | enrollment |
| therapy on patient EFS. | |
| To describe the toxicity of HDC (BuMel) | Toxicity profile of HDC with BuMel after |
| after receiving induction chemotherapy | receiving induction chemotherapy |
| combined with dinutuximab beta. | combined with dinutuximab beta |
| To determine the end-of-infusion drug | Proportion of patients who achieve an |
| level of dinutuximab beta in combination | immunologically active dinutuximab beta |
| with induction chemotherapy. | drug concentration level >1 μg/mL at the |
| | end-of-infusion of the first combination |
| | treatment cycle infusion. |
| To determine the proportion of patients | Proportion of patients who achieve an |
| who achieve an increase in CDC and | increase in CDC and ADCC response |
| ADCC response above baseline against | above baseline against LAN-1 target cells |
| LAN-1 target cells. | at the end-of-infusion of the first |
| | combination cycle (GPOH Cycle 2 and |
| | COJEC Cycle 3). |
| | Comparison of CDC, ADCC and immune |
| | phenotype at baseline and at end of |
| | dinutuximab beta infusion in all |
| | dinutuximab beta cycles. |
| To determine the proportion of patients | Proportion of patients with an increased |
| who have an increased HACA response. | HACA response (end of induction/before |
| | maintenance). |
| | HACA: end of induction (just before HDC |
| | or 4 to 6 weeks after the last |
| | dinutuximab beta dose) and end-of- |
| | study (before maintenance). |

AE = adverse events; ADCC = antibody-dependent cellular cytotoxicity; ASCT = autologous stem cell transplantation; BuMel = busulfan and melphalan; CDC = complement-dependent cytotoxicity; COJEC = cisplatin, vincristine, carboplatin, etoposide, and cyclophosphamide; CR = complete response; DLT = dose-limiting toxicity; EFS = event-free survival; FDG = [¹⁸F]-fluorodeoxyglucose; GPOH = German Pediatric Oncology and Hematology; HACA = human anti-chimeric antibody; HDC = high-dose chemotherapy; INRC = International Neuroblastoma Response Criteria; INRGSS = International Neuroblastoma Risk Group Staging System; mCR = metastatic complete response; MD = minimal disease; MIBG = (¹²³I)-metalodobenzylguanidine; mPR = metastatic partial response rate; MTD = maximum tolerated dose; OS = overall survival; PET = positron emission tomography; SAE = serious adverse events; SIOPEN = International Society of Pediatric Oncology European Neuroblastoma research network.

### Overall Design:

This study is a multicenter, open-label, dual-cohort, Phase 1 study of dinutuximab beta combined with 1 of 2 different induction chemotherapy regimens (GPOH or COJEC) in 2 cohorts of up to 25 evaluable patients each. Newly diagnosed high-risk neuroblastoma patients as defined by Stage M, according to the International Neuroblastoma Risk Group Staging System (INRGSS), age 218 months and <18 years will be included in the trial.

The planned cumulative dinutuximab beta doses (mg/m²) are based on the variable number of treatment days of GPOH or COJEC. When the recommended cumulative dinutuximab beta dose level has been defined, a confirmation cohort of 10 evaluable patients per cohort may be enrolled. The maximum number of patients to be enrolled in the dose escalation and dose confirmation parts of the study combined will be 35 evaluable patients for each induction chemotherapy regimen.

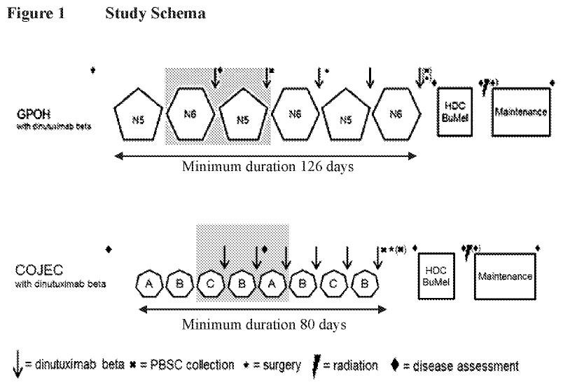

The dose escalation and de-escalation process, including the number of patients dosed, process will follow a Bayesian Optimal Interval (BOIN) design to determine a recommended cumulative dinutuximab beta dose level as new patients enroll. Assessment of toxicity to decide dinutuximab beta dose escalation and de-escalation will occur over the first 2 (GPOH) or 3 (COJEC) induction chemotherapy cycles that are combined with dinutuximab beta (FIG. 1). Two cumulative dose level escalations and 1 cumulative dose level de-escalation are planned.

The data monitoring committee (DMC) will be responsible for safety oversight throughout the study and for dose selection and modification decisions.

The recommended Phase 2 dose (RP2D) will be determined by the maximum tolerated dose (MTD) or the maximum administered cumulative dose level if no MTD dose is reached.

### Number of Investigators and Study Centers:

Approximately 15 Investigators and study centers are expected to participate in this study.

### Number of Patients:

The maximum number of patients to be enrolled in the dose escalation and dose confirmation parts of the study combined will be 35 evaluable patients for each induction chemotherapy regimen (GPOH or COJEC).

### Treatment Groups and Duration:

For each patient, there will be a screening period of up to 21 days, a treatment period consisting of approximately 126 days (GPOH cohort) or 80 days (COJEC cohort), and an end-of-treatment visit at the end of induction treatment. The end-of-study is 100 days after high-dose chemotherapy (HDC)/autologous stem cell transplant (ASCT) or the patient has started a new neuroblastoma treatment instead of HDC/ASCT, whichever is earlier. The planned total duration of the study for each patient enrolled is approximately 3 years.

Dinutuximab beta will be administered at a fixed daily dose of 10 mg/m² given as a 24-hour continuous infusion a scheduled number of days within each treatment cycle (Table 10 and Table 11). The combination of dinutuximab beta with chemotherapy cycles will start in the second (GPOH cohort in combination with dinutuximab beta) or third (COJEC cohort combined with dinutuximab beta) chemotherapy cycle. Patients in the GPOH cohort will receive a total of 5 dinutuximab beta infusions and patients in the COJEC cohort will received a total of 6 dinutuximab beta infusions with their scheduled chemotherapy cycles.

TABLE 4
Statistical Methods

| Analysis Set | Description |
|---|---|
| Screened Analysis Set | All patients who sign the informed consent form (ICF). |
| Enrolled Study Treatment | All patients in the Screened Analysis Set who are assigned to |
| Analysis Set | study treatment. |
| Full Efficacy Analysis Set | All enrolled patients who receive at least 1 dose of dinutuximab |
| | beta and have specific postbaseline efficacy assessments |
| | available. |
| Safety Analysis Set | All enrolled patients who receive at least 1 dose of study |
| | treatment (ie, chemotherapy) will be included in the analysis |
| | of safety parameters and immunogenicity. Patients will be |
| | analyzed according to the treatment they received. |
| Pharmacokinetic (PK) | Patients in the safety analysis set who have at least 1 |
| Analysis Set | quantifiable PK concentration available at a scheduled time |
| | postdose, which is not expected to be significantly affected by |
| | an important protocol deviation/violation or event. |

The sample size is not based on statistical considerations but is typical for studies of this nature and is considered adequate to characterize the distribution of the planned endpoints. Any statistical testing will be considered exploratory and descriptive. All proportions will be estimated with 95% confidence intervals.

### Data Monitoring Committee: Yes

### 1.1 Schedule of Activities

Study procedures and their timing are summarized in Table 5 (GPOH) and Table 6 (COJEC).

TABLE 5
Schedule of Activities: GPOH Induction with Dinutuximab Beta

| | | Pre- | | | | | | | | | | | | | | | | | | |
|---|---|---|---|---|---|---|---|---|---|---|---|---|---|---|---|---|---|---|---|---|
| | | cycle | | | | | | | | | | | | | | | | Mid- | | |
| | | Safety | Cycle N5, N6¹⁸ | | | | | | | | | | | | | | | Induction | End of | End of |
| Day | Scr | Tests ¹⁴ | 1 | 2 | 3 | 4 | 5 | 6 | 7 | 8 | 9 | 10 | 11 | 12 | 13 | 14 | 21 | evaluation | treatment ¹⁵ | study ¹⁶ |
| Obtain informed | X | | | | | | | | | | | | | | | | | | | |
| consent and | | | | | | | | | | | | | | | | | | | | |
| assent¹⁷ | | | | | | | | | | | | | | | | | | | | |
| Eligibility - | X | X | | | | | | | | | | | | | | | | | | |
| Inclusion and | | | | | | | | | | | | | | | | | | | | |
| Exclusion criteria | | | | | | | | | | | | | | | | | | | | |
| Demographics | X | | | | | | | | | | | | | | | | | | | |
| ¹²³I-MIBG SPECT¹ | X | | | | | | | | | | | | | | | | | X | X | X |
| MRI/CT (brain and | X | | | | | | | | | | | | | | | | | | X | X |
| primary tumor) | | | | | | | | | | | | | | | | | | | | |
| Two bone marrow | X | | | | | | | | | | | | | | | | | X | X | X |
| aspirates² | | | | | | | | | | | | | | | | | | | | |
| Two bone marrow | X | | | | | | | | | | | | | | | | | X | X | X |
| trephine biopsies | | | | | | | | | | | | | | | | | | | | |
| Urinary | X | | | | | | | | | | | | | | | | | X | X | X |
| catecholamine | | | | | | | | | | | | | | | | | | | | |
| metabolites | | | | | | | | | | | | | | | | | | | | |
| Tumor biopsy and | X | | | | | | | | | | | | | | | | | | | |
| histology³ | | | | | | | | | | | | | | | | | | | | |
| LDH, ferritin | X | | | | | | | | | | | | | | | | | | | |
| MYCN | X | | | | | | | | | | | | | | | | | | | |
| amplification⁴ | | | | | | | | | | | | | | | | | | | | |
| Pregnancy test¹³ | X | X | | | | | | | | | | | | | | | | | X | X |
| HIV/HbsAg/ | X | | | | | | | | | | | | | | | | | | | |
| Hepatitis C | | | | | | | | | | | | | | | | | | | | |
| Chemotherapy⁵ | | | X | X | X | X | X | X | X | X | | | | | | | | | | |
| Dinutuximab beta⁶ | | | | X | X | X | X | X | X | X | | | | | | | | | | |
| G-CSF¹⁹ | | | | | | | | | | | X | X | X | X | X | X | X | | | |
| Complete medical | X | | | | | | | | | | | | | | | | | | | |
| history | | | | | | | | | | | | | | | | | | | | |
| Physical | X | X | X | X | X | X | X | X | X | X | X | | | | | | | X | X | X |
| examination/vital | | | | | | | | | | | | | | | | | | | | |
| signs/weight | | | | | | | | | | | | | | | | | | | | |
| Height | X | | | | | | | | | | | | | | | | | | | |
| Performance | X | X | X | X | X | X | X | X | X | X | X | | | | | | | X | X | X |
| status | | | | | | | | | | | | | | | | | | | | |
| Pain assessment | X | X | X | X | X | X | X | X | X | X | X | | | | | | | | | |
| Adverse events | X | X | X | X | X | X | X | X | X | X | X | X | X | X | X | X | X | X | X | X |
| Concomitant | X | X | X | X | X | X | X | X | X | X | | | | | | | | X | X | X |
| medication¹² | | | | | | | | | | | | | | | | | | | | |
| Echocardiography⁷ | X | X | X | | | | | | | | | | | | | | | X | X | X |
| 12-lead ECG⁷ | X | X | X | | | | | | | | | | | | | | | | X | X |
| Hearing test⁹ | X | X | X | | | | | | | | | | | | | | | | X | X |
| PFT/O₂ saturation | X | | | | | | | | | | | | | | | | | | X | X |
| on room air | | | | | | | | | | | | | | | | | | | | |
| Full blood count | X | X | X | X⁸ | | | | | | X⁸ | | | | | | | X | X | X | X |
| and biochemistry^{8, 10} | | | | | | | | | | | | | | | | | | | | |
| Creatinine and/or | X | X | X | | | | | | | | | | | | | | | | X | X |
| cystatin C | | | | | | | | | | | | | | | | | | | | |
| clearance¹¹ | | | | | | | | | | | | | | | | | | | | |
| Immunophenotyping⁸ | X | | | X⁸ | | | | | | X⁸ | | | | | | | | | | |
| Genotyping, | X | | | | | | | | | | | | | | | | | | | |
| polymorphisms, | | | | | | | | | | | | | | | | | | | | |
| KIR/KIR-ligand | | | | | | | | | | | | | | | | | | | | |
| mismatch | | | | | | | | | | | | | | | | | | | | |
| PK⁸ | | | | X⁸ | | | | | | X⁸ | | | | | | | | | | |
| HACA | × | | | X | | | | | | | | | | | | | | | X | X |
| CDC and ADCC⁸ | X | | | X⁸ | | | | | | X⁸ | | | | | | | | | | |

Abbreviations: ADCC = antibody-dependent cellular cytotoxicity; ALT = alanine aminotransferase; ASCT = autologous stem cell transplantation; AST = aspartate aminotransferase; CDC = complement-dependent cytotoxicity; CRP = c-reactive protein; CT = computed tomography; ECG = electrocardiogram; GPOH = German Pediatric Oncology and Hematology; HACA = human anti-chimeric antibody; G-CFS = granulocyte-colony stimulating factor; HDC = high-dose chemotherapy; HbsAg = hepatitis B surface antigen; HIV = human immunodeficiency virus; INPC = International Neuroblastoma Pathology Classification; KIR = killer immunoglobulin-like receptor; ¹²³I-MIBG = ¹²³I-metaiodobenzylguanidine; LDH = lactate dehydrogenase; MRI = magnetic resonance imaging; O₂ = oxygen; PFT = pulmonary function test; PET = positron emission tomography; PK = pharmacokinetic; Scr = screening; SPECT = single-photon emission tomography.
¹Replace by [¹⁸F]-fluorodeoxyglucose-PET in patients with MIBG-negative neuroblastoma.
²Assessment by light microscopy, by immunocytology. Quantitative reverse-transcriptase polymerase chain reaction optional.
³Histology and assessment according to INPC (Shimada-Classification) (1).
⁴MYCN status can be determined in tumor biopsy material or infiltrated bone marrow samples.
⁵Treatment days of chemotherapy vary according to assigned cycle. Maximum days are indicated. For additional information refer to Section 0.
⁶Treatment days of dinutuximab beta vary according to assigned dose level. Maximum days indicated. For additional information refer to Section 0. Details of start and stop times, interruptions (stop/restart), flow rate, date and time of infusion preparation changeover, etc, are to be included in the case report form (CRF).
⁷Repeat echocardiography prior to each N6 - chemotherapy element to capture anthracycline mediated cardiotoxicity.
⁸Sample to be collected on first day of dinutuximab beta administration (prior to start of infusion) and on the last day of dinutuximab beta administration (after the end of the infusion). Note that the Cycle days will depend on the cumulative infusion dose identified in Table 8, Table 10, and Section 6.2.1; the earliest/latest days are indicated. See Table 12 for details of blood sample collection times and collection time windows.
⁹Repeat hearing test every 2 N5 cycle- chemotherapy element to capture ototoxicity.
¹⁰Full blood count: hemoglobin, platelets, white blood cells with differential (absolute neutrophils, absolute lymphocytes, absolute eosinophils, absolute basophils) Biochemistry: sodium, potassium, chloride, ALT, AST, total bilirubin, creatinine and/or cystatin C, and CRP.
¹¹Creatinine and/or cystatin C clearance has to be performed before each chemotherapy cycle to capture nephrotoxicity.
¹²Documentation required.
¹³Pregnancy test is only required in female patients of childbearing potential.
¹⁴ Tests prior to the start of each chemotherapy cycle contain safety parameters that may lead to change in dosing of chemotherapy and must be performed in advance. These assessments should be available prior to first dosing of chemotherapy on Day 1. Information can be collected up to 96 hours prior to first dosing.
¹⁵ End-of-treatment corresponds to end of induction, clinic visit may be conducted up to 4 weeks after end of induction. For patients who do not continue to high-dose chemotherapy after induction and proceed with alternative therapy due to refractory disease, the end of induction visit will also serve as the end-of-study visit. Post-study cancer treatment, including but not limited to maintenance treatment, details (normally type and amount/duration of cancer treatment) will be recorded.
¹⁶ End-of-study is 100 days after HDC/ASCT or has started new neuroblastoma treatment instead of HDC/ASCT, whichever is earlier. Clinic visit may be up to 4 weeks after EOS.
¹⁷Consent and assent will be obtained according to local regulatory guidelines for pediatric patients.
¹⁸Activities on Days 1 to 21 are repeated for each treatment cycle, starting with treatment cycle 2 when dinutuximab beta is initiated.
¹⁹Timepoints indicate the maximum duration of G-CSF.

TABLE 6
Schedule of Activities: COJEC Induction with Dinutuximab Beta

| | | Pre- | | | | | | | | | | | |
|---|---|---|---|---|---|---|---|---|---|---|---|---|---|
| | | cycle | | | | | | | | | Mid- | | |
| | | Safety | Cycle A, B, C¹⁷ | | | | | | | | Induction | End of | End of |
| Day | Screening | Tests¹³ | 1 | 2 | 3 | 4 | 5 | 6 | 7 | 8 | evaluation | treatment¹⁴ | study¹⁵ |
| Obtain informed consent and | X | | | | | | | | | | | | |
| assent¹⁶ | | | | | | | | | | | | | |
| Eligibility - Inclusion and | X | | | | | | | | | | | | |
| Exclusion criteria | | | | | | | | | | | | | |
| ¹²³I-MIBG SPECT ¹ | X | | | | | | | | | | X | X | X |
| MRI/CT (brain and primary | X | | | | | | | | | | | X | X |
| tumor) | | | | | | | | | | | | | |
| Two bone marrow aspirates² | X | | | | | | | | | | X | X | X |
| Two bone marrow trephine | X | | | | | | | | | | X | X | X |
| biopsies | | | | | | | | | | | | | |
| Urinary catecholamine | X | | | | | | | | | | X | X | X |
| metabolites | | | | | | | | | | | | | |
| Tumor biopsy and histology³ | X | | | | | | | | | | | | |
| LDH, ferritin | X | | | | | | | | | | | | |
| MYCN amplification⁴ | X | | | | | | | | | | | | |
| Pregnancy test¹² | X | X | | | | | | | | | | X | X |
| HIV/HbsAg | X | | | | | | | | | | | | |
| Chemotherapy⁵ | | | X | X | | | | | | | | | |
| Dinutuximab beta⁶ | | | X | X | X | X | X | | | | | | |
| G-CSF¹⁸ | | | | X | X | X | X | X | X | X | | | |
| Complete medical history | X | | | | | | | | | | | | |
| Physical examination/vital | X | X | X | X | X | X | X | | | | X | X | X |
| signs/weight | | | | | | | | | | | | | |
| Height | X | | | | | | | | | | | X | X |
| Performance status | X | X | X | X | X | X | X | | | | X | X | X |
| Pain assessment | X | X | X | X | X | X | X | | | | | | |
| Adverse events | | X | X | X | X | X | X | X | X | X | X | X | X |
| Concomitant medication¹¹ | X | X | X | X | X | X | X | X | X | X | X | X | X |
| Echocardiography | X | | | | | | | | | | X | X | X |
| 12-lead ECG | X | | | | | | | | | | | X | X |
| Hearing test⁸ | X | X | | | | | | | | | | X | X |
| PFT/O₂ saturation on room | X | | | | | | | | | | | X | X |
| air | | | | | | | | | | | | | |
| Full blood count and | X | X | X⁷ | | | | X⁷ | | | X | X | X | X |
| biochemistry^{7, 9} | | | | | | | | | | | | | |
| Creatinine and/or cystatin C | X | X | | | | | | | | | X | X | X |
| clearance¹¹ | | | | | | | | | | | | | |
| Immunophenotyping⁷ | X | | X⁷ | | | | X⁷ | | | | | | |
| Genotyping, polymorphisms, | X | | | | | | | | | | | | |
| KIR/KIR-ligand mismatch | | | | | | | | | | | | | |
| PK⁷ | X | | X⁷ | | | | X⁷ | | | | | | |
| HACA | X | | X | | | | | | | | | X | X |
| CDC and ADCC⁷ | X | | X⁷ | | | | X⁷ | | | | | | |

Abbreviations: ADCC = antibody-dependent cellular cytotoxicity; ALT = alanine aminotransferase; ASCT = autologous stem cell transplantation; AST = aspartate aminotransferase; CDC = complement-dependent cytotoxicity; COJEC = cisplatin, vincristine, carboplatin, etoposide, and cyclophosphamide; CRF = case report form; CRP = c-reactive protein; CT = computed tomography; ECG = electrocardiogram; G-CFS = granulocyte-colony stimulating factor; HACA = human anti-chimeric antibody; HDC = high-dose chemotherapy; HbsAg = hepatitis B surface antigen; HIV = human immunodeficiency virus; INPC = International Neuroblastoma Pathology Classification; KIR = killer immunoglobulin-like receptor; ¹²³I-MIBG = ¹²³I-metaiodobenzylguanidine; LDH = lactate dehydrogenase; MRI = magnetic resonance imaging; O₂ = oxygen; PFT = pulmonary function test; PET = positron emission tomography; PK = pharmacokinetic; Scr = screening; SPECT = single-photon emission tomography.
¹ Replace by [¹⁸F]-fluorodeoxyglucose-PET in patients with MIBG-negative neuroblastoma.
²Assessment by light microscopy, by immunocytology. Quantitative reverse-transcriptase polymerase chain reaction optional.
³Histology and assessment according to INPC (Shimada-Classification) (1).
⁴MYCN status can be determined in tumor biopsy material or infiltrated bone marrow samples.
⁵Treatment days of chemotherapy vary according to assigned cycle. Maximum days are indicated. For additional information see Section 0.
⁶Treatment days of dinutuximab beta vary according to assigned dose level. Maximum days indicated. For additional information see Section 0. Details of start and stop times, interruptions (stop/restart), flow rate, date and time of infusion preparation changeover, etc, are to be included in the CRF.
⁷Sample to be collected on first day of dinutuximab beta administration (prior to start of infusion) and on the last day of dinutuximab beta administration (after the end of the infusion). Note that the Cycle days will depend on the cumulative infusion dose identified in Table 8, Table 11, and Section 6.2.2 the earliest/latest days are indicated. See Table 12 for details of PK blood sample collection times and collection time windows.
⁸Repeat hearing test prior to every other B-chemotherapy element to capture ototoxicity.
⁹Full blood count: hemoglobin, platelets, white blood cells with differential (absolute neutrophils, absolute lymphocytes, absolute eosinophils, absolute basophils). Biochemistry: sodium, potassium, chloride, ALT, AST, total bilirubin, creatinine and/or cystatin C, and CRP.
10Creatinine and/or cystatin C clearance has to be performed before each chemotherapy cycle to capture nephrotoxicity.
¹¹Documentation required.
¹²Pregnancy test is only required in female patients of childbearing potential.
¹³ Tests prior to the start of all chemotherapy cycles contain safety parameters the may lead to change in dosing of chemotherapy.. These assessments should be available prior to first dosing of chemotherapy on Day 1. Information can be collected up to 96 hours prior to first dosing.
¹⁴End-of-treatment corresponds to end of induction, clinic visit may be conducted up to 4 weeks after end of induction. For patients who do not continue to high-dose chemotherapy after induction and proceed with alternative therapy due to refractory disease, the end of induction visit will also serve as the end-of-study visit. Post-study cancer treatment, including but not limited to maintenance treatment, details (normally type and amount/duration of cancer treatment) will be recorded.
¹⁵End-of-study is 100 days after HDC/ASCT or has started new neuroblastoma treatment instead of HDC/ASCT, whichever is earlier. Clinic visit may be up to 4 weeks after EOS.
¹⁶Consent and assent will be obtained according to local regulatory guidelines for pediatric patients.
¹⁷Activities on Days 1 to 21 are repeated for each treatment cycle, starting with treatment Cycle 3 when dinutuximab beta is initiated.
¹⁸Sample collection days indicate the maximum duration of G-CSF measurement.

### 2.0 Introduction

### 2.1 Study Rationale

This study is designed to identify safe and effective infusion durations for and cumulative dose levels of dinutuximab beta when combined with different induction chemotherapy regimens (German Pediatric Oncology and Hematology [GPOH] or cisplatin, vincristine, carboplatin, etoposide, and cyclophosphamide [COJEC]) for the treatment of newly diagnosed patients with high-risk neuroblastoma without substantially exceeding the treatment density previously established for dinutuximab beta monotherapy. The information will be used in the planning of future studies of an established Induction chemotherapy regimen randomized to GPOH or COJEC with or without dinutuximab beta.

### 2.2 Background

Neuroblastoma, the most common extracranial solid tumor in children, remains one of the major challenges in pediatric oncology. Despite the introduction of novel treatment strategies, the outcomes of patients with high-risk neuroblastoma remain poor [2,3], and new effective adjuvant therapeutics are desperately needed to further improve clinical outcomes in these patients.

Dinutuximab beta is a chimeric monoclonal antibody produced in Chinese hamster ovary cells (CHO) targeting the dislaloganglioside GD2 antigen highly expressed by neuroectodermal tumors such as neuroblastoma, melanoma cells, and several other tumors. Dinutuximab beta is licensed in the European Union (EU) subject to additional monitoring as Qarziba administered at 10 mg/m²/day as an 8-hour or 24-hour infusion [4]. Dinutuximab beta is indicated for the treatment of high-risk neuroblastoma in patients aged 12 months and above, who have previously received induction chemotherapy and achieved at least a partial response, followed by myeloablative therapy and stem cell transplantation, as well as patients with history of relapsed or refractory neuroblastoma, with or without residual disease. Prior to the treatment of relapsed neuroblastoma, any actively progressing disease should be stabilized by other suitable measures.

Over the last 4 decades, different chemotherapy regimens have been evaluated in this treatment setting by academic cooperative groups (GPOH, International Society of Pediatric Oncology European Neuroblastoma research network [SIOPEN], and the Children's Oncology Group [COG]) with increasing intensity and different combinations of conventional chemotherapeutics. Most induction chemotherapy regimens include platinum compounds (cisplatin and/or carboplatin), cyclophosphamide, etoposide, and vincristine, topoisomerase inhibitors (topotecan), and anthracyclines.

Multiple reports have demonstrated that achieving a complete response (CR) after induction is one of the most powerful predictors of outcome. This was shown in a study by the European Bone Marrow Transplantation registry where achieving CR before high-dose chemotherapy was an independent predictor of event-free survival (EFS) and overall survival (OS) in multivariable analysis (5).

Chemotherapy regimens have been developed and optimized by the GPOH over 4 decades in patients with high-risk neuroblastoma patients. This development resulted in the use of 6 alternating chemotherapy cycles of N5 (cisplatin, etoposide, and vindesine) and N6 (ifosfamide, vincristine, dacarbazine, and doxorubicin). That is the standard induction therapy in Germany since 1997 (6). Surgery is performed during the induction treatment. Complete (CR) and very good partial responses are achieved in 55% patients.

The current guideline for the treatment of children with high-risk neuroblastoma in Germany refers to the N5/N6 Induction chemotherapy (7).

In 2013, an additional randomization (R3) was introduced into the SIOPEN HR-NBL1 study, to compare COJEC with the modified N7 Induction chemotherapy regimen (8 to 10), developed at Memorial Sloan-Kettering Cancer Center; this regimen had also been adopted by the COG. This is a dose-intensive induction chemotherapy regimen including 2 putatively non-cross-resistant drug combinations: high-dose cyclophosphamide plus doxorubicin/vincristine and high-dose cisplatin/etoposide (PIE). The original regimen with 7 cycles was modified reducing the number to 5 cycles, with a lower dosage of vincristine (VCR) and using granulocyte-colony-stimulating factor (G-CSF). A planned interim analysis after recruitment of the last patient was performed in September 2017, and an early data release was authorized by the data monitoring committee (DMC) for study planning purposes. The results show a complete overlap of EFS and OS curves of both arms, but a better toxicity profile for in the COJEC treatment arm. Therefore, COJEC is maintained as the standard treatment arm for future study planning.

Therefore, the assessment of the feasibility of combining dinutuximab beta with GPOH or COJEC induction chemotherapy regimens and identifying recommended infusion durations and cumulative dose levels of dinutuximab beta for each of these combination chemotherapy regimens will be investigated in this Phase 1 study. The inclusion of expansion cohorts is to provide additional safety and efficacy data to support the selection of the maximum tolerated dose (MTD) and add confidence for future study designs. The data will be used to further inform future study designs to evaluate the GPOH or COJEC induction chemotherapy regimens with or without dinutuximab beta.

### 2.3 Benefit/Risk Assessment

The potential benefit of using dinutuximab beta during induction chemotherapy is unknown. However, in a recently reported Phase 2 study of hu14.18K322A (humanized anti-GD2 monoclonal antibody) it was shown that adding hu14.18K322A to induction chemotherapy produced early partial responses, or better, in most patients with newly diagnosed high-risk neuroblastoma. There was also reduced tumor volumes, Improved (¹²³I) metaiodobenzylguanidine (MIBG) scores at the end of induction, and an encouraging 2-year event-free survival (11). Special warnings and precautions for dinutuximab beta include pain, hypersensitivity reactions, neurological disorders of the eye, peripheral neuropathy, capillary leak syndrome, systemic infections, hematologic toxicities, and laboratory abnormalities (liver function and electrolytes). The development of symptoms of these events will be closely monitored by the investigators during the study. Actions to be taken in the event of specific product-related toxicities development of symptoms are described in Appendix 4.

Infusion of dinutuximab beta must be initiated in an environment where full resuscitation services are immediately available Section 6.6.1.

The development of antidrug antibodies is a class effect of monoclonal chimeric antibodies. However, no apparent influence of these neutralizing antibodies on safety and efficacy has been observed in clinical studies. Blood samples will be collected during the study to measure human anti-chimeric antibody (HACA).

There is no data dinutuximab beta safety data in pregnant women. Due to the expression of the dinutuximab beta target (GD2) on neuronal tissues especially during embryofetal development, the cytotoxic potential of dinutuximab beta and the potential of placental transfer of antibodies, dinutuximab beta may cause fetal harm when administered to pregnant women. Female patients of childbearing potential will be required to have a negative pregnancy test before enrollment, pregnancy tests will be conducted during the study and the patients will be required to agree to follow contraceptive guidance.

As no drug-drug interaction studies have been performed with dinutuximab beta, the effect of dinutuximab beta on any other drugs that the child might take cannot be predicted. Prohibited concomitant medications on this study are described in Appendix 2.

The safety and efficacy of dinutuximab beta in children aged <12 months have not yet been established and no data are available. Patients enrolled in the study will be aged between ≥18 months and <18 years.

As only patients with adequate renal and hepatic function have been investigated to date, patient enrollment will be restricted to those with confirmed adequate renal and kidney function.

More detailed information about the known and expected benefits and risks and reasonably expected adverse events (AEs) of dinutuximab beta may be found in the Investigator's Brochure.

The Sponsor will immediately notify the investigators if any additional safety or toxicology information becomes available during the study.

This study will be performed in compliance with the protocol, International Council for Harmonisation of Technical Requirements for Pharmaceuticals for Human Use (ICH) Good Clinical Practice (GCP), and applicable regulatory requirements. Aspects of the study concerned with the investigational medicinal product(s) will meet the requirements of European Union—Good Manufacturing Practice (EU GMP).

### 3 Objectives and Endpoints

TABLE 7
Study Objectives and Endpoints

| Objectives | Endpoints |
|---|---|
| Primary | |
| To assess the safety and tolerability | Incidences of DLTs associated with the |
| and identify the MTDs of dinutuximab | combination of dinutuximab beta with |
| beta when combined with different | GPOH or COJEC induction |
| induction chemotherapy regimens | chemotherapy regimens. |
| (GPOH or COJEC) for the treatment of | |
| newly diagnosed high-risk | |
| neuroblastoma patients ≥18 months of | |
| age as defined by Stage M, according | |
| to the INRGSS. | |
| Secondary | |
| To characterize the toxicity of induction | Type, incidence, severity, seriousness |
| chemotherapy when combined with | and relationship to study medications |
| dinutuximab beta. | for AES, including laboratory |
| | abnormalities and SAEs. |
| | Cumulative incidence of treatment- |
| | related mortality and of disease-related |
| | mortality. |
| To determine the overall response | Overall response during and after |
| (primary tumor, metastases) at the | induction (primary tumor, metastases). |
| end of induction chemotherapy with | |
| dinutuximab beta. | |
| To determine the metastatic response | mCR after 2 cycles GPOH and after |
| rate in response to chemotherapy plus | 4 cycles COJEC. |
| dinutuximab beta. | mCR after induction treatment. |
| | mPR according to eligibility criteria to |
| | proceed to consolidation by HDC/ASCT: |
| | mPR for bone disease: MIBG |
| | uptake (or FDG-PET uptake for |
| | MIBG-nonavid tumors) |
| | completely resolved or SIOPEN |
| | score ≤3 and at least 50% |
| | reduction in MIBG score (or ≤3 |
| | bone lesions and at least 50% |
| | reduction in number of |
| | FDG-PET-avid bone lesions for |
| | MIBG-nonavid tumors). |
| | mPR for bone marrow disease: |
| | CR and/or MD according to |
| | INRC. |
| | mPR for other metastatic sites: |
| | complete response after |
| | induction chemotherapy +/− |
| | surgery. |
| Exploratory | |
| To determine the impact of GPOH or | 3-year and 5-year OS, calculated from |
| COJEC with dinutuximab beta induction | date of enrollment to the date of last |
| therapy on patient OS. | follow-up or death from any cause. |
| To determine the impact of GPOH or | 3-year and 5-year EFS from the date of |
| COJEC with dinutuximab beta induction | enrollment |
| therapy on patient EFS. | |
| To describe the toxicity of HDC (BuMel) | Toxicity profile of HDC with BuMel after |
| after receiving induction chemotherapy | receiving induction chemotherapy |
| combined with dinutuximab beta. | combined with dinutuximab beta |
| To determine the end-of-infusion drug | Proportion of patients who achieve an |
| level of dinutuximab beta in | immunologically active dinutuximab |
| combination with induction | beta drug concentration level >1 μg/mL |
| chemotherapy. | at the end-of-infusion of first |
| | combination treatment cycle infusion. |
| To determine the proportion of patients | Proportion of patients who achieve an |
| who achieve an increase in CDC and | increase in CDC and ADCC response |
| ADCC response above baseline against | above baseline against LAN-1 target |
| LAN-1 target cells. | cells at the end-of-infusion of the first |
| | combination cycle (GPOH Cycle 2 and |
| | COJEC Cycle 3). |
| | Comparison of CDC, ADCC and immune |
| | phenotype at baseline and at end of |
| | dinutuximab beta infusion in all |
| | dinutuximab beta cycles. |
| To determine the proportion of patients | Proportion of patients with an increased |
| who have an increased HACA response. | HACA response (end of |
| | induction/before maintenance). |
| | HACA: end of induction (just before |
| | HDC or 4 to 6 weeks after the last |
| | dinutuximab beta dose) and end-of- |
| | study (before maintenance). |

AE = adverse events; ADCC = antibody-dependent cellular cytotoxicity; ASCT = autologous stem cell transplantation; BuMel = busulfan and melphalan; CDC = complement-dependent cytotoxicity; COJEC = cisplatin, vincristine, carboplatin, etoposide, and cyclophosphamide; CR = complete response; DLT = dose-limiting toxicity; EFS = event-free survival; FDG = [¹⁸F]-fluorodeoxyglucose; GPOH = German Pediatric Oncology and Hematology; HACA = human anti-chimeric antibody; HDC = high-dose chemotherapy; INRC = International Neuroblastoma Response Criteria; INRGSS = International Neuroblastoma Risk Group Staging System; mCR = metastatic complete response; MD = minimal disease; MIBG = (¹²³I) metalodobenzylguanidine; mPR = metastatic partial response rate; MTD = maximum tolerated dose; OS = overall survival; PET = positron emission tomography; SAE = serious adverse events; SIOPEN = International Society of Pediatric Oncology European Neuroblastoma research network.

### 4 Study Design

### 4.1 Overall Design

This study is a multicenter, open-label, dual-cohort, Phase 1 study of dinutuximab beta combined with 2 different induction chemotherapy regimens (GPOH or COJEC) in 2 cohorts of up to 25 evaluable patients each. Study centers will enroll patients in 1 cohort at a time (ie, only recruit patients scheduled to receive the GPOH or COJEC induction chemotherapy regimen); the allocation of study centers to a treatment cohort may change during the study depending on the recruitment per cohort. If one induction chemotherapy regimen cohort has completed recruitment, then centers may activate the other induction chemotherapy regimen cohort in order to contribute to the recruitment of the slower recruiting cohort after approval by the Sponsor or Medical Monitor. When the recommended cumulative dinutuximab beta dose level has been defined, a confirmation cohort of 10 evaluable patients per cohort may be enrolled. The maximum number of patients to be enrolled in the dose escalation and dose confirmation parts of the study combined will be 35 evaluable patients for each induction chemotherapy regimen.

Newly diagnosed high-risk neuroblastoma patients as defined by Stage M, according to the International Neuroblastoma Risk Group Staging System (INRGSS), age ≥18 months and <18 years will be included in the trial.

For each patient, there will be a screening period of up to 21 days, a treatment period consisting of approximately 126 days (GPOH cohort) or 80 days (COJEC cohort), an end-of-treatment visit at the end of induction treatment, and an end-of-study follow-up period 100 days after high-dose chemotherapy (HDC)/autologous stem cell transplant (ASCT) performed as standard-of-care. The planned total duration of the study for each patient enrolled is approximately 3 years. Assessments performed during the study are specified in Table 5 (COJEC) and Table 6 (GPOH).

The GPOH induction chemotherapy regimen will be administered in 21-day cycles (total of 6 cycles). The combination of dinutuximab beta with induction chemotherapy cycles will start in the second chemotherapy cycle (FIG. 1). Dinutuximab beta will be administered at a fixed daily dose of 10 mg/m² given as a 24-hour continuous infusion a scheduled number of days within each treatment cycle (Table 10).

The COJEC induction chemotherapy will be administered over 10 weeks in 10-day cycles (total of 8 cycles). The combination of dinutuximab beta with chemotherapy cycles will start in the third chemotherapy cycle (FIG. 1). Dinutuximab beta will be administered at a fixed daily dose of 10 mg/m² given as a 24-hour continuous infusion a scheduled number of days within each treatment cycle (Table 11) for a total of 6 Infusions.

Cumulative dose levels are defined by the total amount of dinutuximab beta the patient is expected to receive over the entire course of therapy. A higher cumulative dose levels reflect that the patient will receive more total days of therapy (although not higher infusion rates).

The planned cumulative dinutuximab beta doses (mg/m²) based on the variable number of treatment days of GPOH or COJEC are presented in Table. See Section 0 for more details.

TABLE 8
Planned Cumulative Dinutuximab Beta Doses

| Regimen: | GPOH^{a} | COJEC^{b} |
|---|---|---|
| Number of Combined Courses | 5 | 6 |
| Cumulative Dinutuximab Beta | | |
| Dose Level | | |
| −1 | 190 mg/m² | 140 mg/m² |
| 1 (Starting Dose Level) | 250 mg/m² | 180 mg/m² |
| 2 | 310 mg/m² | 210 mg/m² |
| 3 | 350 mg/m² | 240 mg/m² |

Abbreviations:
COJEC = cisplatin, vincristine, carboplatin, etoposide, and cyclophosphamide
GPOH = German Pediatric Oncology and Hematology
^{a}Refer to Table 10 for details of each 21-day cycle
^{b}Refer to Table 11 for details of each 10-day cycle

The dose escalation and de-escalation process, including the number of patients dosed, will follow a Bayesian Optimal Interval (BOIN) design to determine a recommended cumulative dinutuximab beta dose level as new patients enroll (Section 6.7.3). Assessment of toxicity to decide dinutuximab beta dose escalation or de-escalation will occur over the first 2 (GPOH) or 3 (COJEC) Induction chemotherapy cycles that are combined with dinutuximab beta. The primary DLT observation period (2 Induction chemotherapy cycles for GPOH and 3 Induction chemotherapy cycles for COJEC) is indicated by the gray-shaded area for each regimen in FIG. 1. Dose-limiting toxicity is defined in Section 8.1.1.

Two cumulative dose level escalations and 1 cumulative dose level de-escalation are planned. Details of dinutuximab beta administration and dose escalation/de-escalation rules are described in Section 6.7.3. Dose modification procedures for chemotherapy treatment during dinutuximab beta administration are described in Section 6.2.

The DMC will be responsible for safety oversight and for dose selection and modification decisions.

The recommended Phase 2 dose (RP2D) will be determined by the MTD or the maximum administered cumulative dose level if no MTD dose is reached.

### 4.2 Scientific Rationale for Study Design

Neuroblastoma, the most common pediatric extracranial tumor, remains a leading cause of death from cancer in children. Long-term survival of children with high-risk neuroblastoma remains below 60% at 5 years despite improvements in intensive multimodal therapy, including chemotherapy, HDC with autologous hematopoletic stem cell rescue, surgical removal of the primary tumor, radiotherapy, residual disease therapy, and immunotherapy with anti-GD2 monoclonal antibodies.

As one of the most powerful prognostic factors in high-risk neuroblastoma is the metastatic response to induction therapy, current efforts to improve the overall outcome for children with high-risk neuroblastoma are concentrated on identifying the optimal induction treatment regimen. The major induction chemotherapy regimens used by cooperative groups (such as GPOH and COJEC) have produced response rates in separate clinical studies response rates ranging between 50% and 75%. The potential benefit of using dinutuximab beta during induction chemotherapy is unknown. Therefore, systematic evaluation of the feasibility and tolerability of dinutuximab beta therapy in combination with established induction chemotherapy regimens for the treatment of previously untreated high-risk neuroblastoma will be investigated in this study.

Before initiating a randomized clinical study, it is necessary to determine an appropriate cumulative dose level of dinutuximab beta in combination with chemotherapy regimens such as GPOH or COJEC. Dinutuximab beta will be given at a fixed daily dose of 10 mg/m² as a continuous infusion. The goal is to identify an appropriate cumulative dose level of dinutuximab beta without substantially exceeding its previously established monotherapy treatment density within each induction chemotherapy regimen for further investigation.

### 4.3 Justification for Dose

### 4.3.1 Rationale for the Starting Dinutuximab Beta Infusion Duration

The starting cumulative dose selected for administration with the GPOH and the COJEC Induction chemotherapy regimens was based on the pharmacokinetic (PK) data following administration of dinutuximab beta for 10 days (100 mg/m²/course) in 35-day intervals (12).

Dinutuximab beta infusion durations for the 2 planned treatment induction chemotherapy regimens:
GPOH: starting dinutuximab beta infusion duration=10 mg/m²×5 days (50 mg/m²/course) in 21-day treatment intervals.
COJEC: starting dinutuximab beta infusion duration=10 mg/m²×3 days (30 mg/m²/course) in 10-day treatment intervals.
   4.3.2 Rationale for the Timing of Dinutuximab Beta Administration in the induction Chemotherapy Regimen

The rationale for delaying the start of dinutuximab beta administration in the induction chemotherapy regimens is to minimize the risk of combined toxicities by decreasing the overlap of chemotherapy and dinutuximab beta. In particular, the combination of potential nephrotoxicity induced by chemotherapeutic agents combined with capillary leak induced by dinutuximab beta was especially considered for safety risk minimization.

Escalating starting infusion durations have an increasing number of days with a simultaneous administration of chemotherapy and dinutuximab beta to accommodate the timing within the cycle which will help determine feasibility in combination with hyperhydration.

4.3.3 Justification for the Dose Schedules within an Entire Induction Regimen

### 4.3.3.1 Rationale for the Starting Dose Schedule

The rationale for the starting cumulative dinutuximab beta dose levels is derived from the planned treatment density of dinutuximab beta within the GPOH or COJEC Induction chemotherapy regimen in comparison to the treatment density used in the SIOPEN monotherapy long-term infusion (LTI) dinutuximab beta study (11).

In monotherapy LTI dinutuximab beta studies (10 days; 10 mg/m²/day; 35-day treatment intervals, 5 cycles), a cumulative dose of 500 mg/m² given in 175 days was identified as feasible and well-tolerated in maintenance treatment. The treatment density was then calculated by dividing the cumulative dose by the duration of maintenance treatment (=2.86 mg/m²×day).

The treatment tolerance is expected to be similar between maintenance and induction, the treatment density of 2.86 mg/m²×day was used as a target range for the planning of the first infusion duration of dinutuximab beta given in combination with the induction chemotherapy regimen GPOH (cumulative dose level 1) and COJEC (cumulative dose level 1).

As Induction chemotherapy regimens vary in length, different cumulative dinutuximab beta starting doses were planned for the first dose level of each cohort. This was done in order to achieve dinutuximab beta treatment densities in each of the 2 cohorts that are in a similar range to the ones previously used in monotherapy LTI studies. The results of that planning are presented in Table 9.

TABLE 9
Cumulative Dinutuximab Beta Starting Doses for the First
Dose Level of Each Induction Chemotherapy Cohort

| | | | | Treatment |
|---|---|---|---|---|
| | | | | Density: |
| | | | | Cumulative |
| | | | | Dose/ |
| | | Duration of | | Duration of |
| | | Induction | Cumulative | Induction |
| | Duration of | Chemotherapy + | Dose of | Chemotherapy + |
| | Induction | Dinutuximab | Dinutuximab | Dinutuximab |
| Treatment | Chemotherapy | Beta | Beta | Beta |
| Monotherapy | 175 days | | 500 mg/m² | 2.86 mg/m²/day |
| LTI Studies | | | | |
| Cumulative | 126 days | 105 days | 250 mg/m² | 2.38 mg/m²/day |
| Dose Level 1 | | | | |
| for GPOH | | | | |
| Cumulative | 80 days | 60 days | 180 mg/m² | 3.00 mg/m²/day |
| Dose Level 1 | | | | |
| for COJEC | | | | |

Abbreviations: COJEC = cisplatin, vincristine, carboplatin, etoposide, and cyclophosphamide; GPOH = German Pediatric Oncology and Hematology; LTI = long-term infusion.

### 4.3.3.2 Dose Schedules Planned for GPOH or COJEC Induction Chemotherapy Regimens

Up to 4 cumulative dose levels are planned for dinutuximab beta in the context of GPOH or COJEC induction chemotherapy regimens. They are defined by varying infusion durations of dinutuximab beta within a chemotherapy cycle. The cumulative dose levels for the GPOH or COJEC Induction chemotherapy regimen are presented in Table 10 and Table 11 respectively.

4.3.3.3 Rationale for the Dinutuximab Beta Dose-Finding Strategy in Combination with the GPOH induction Chemotherapy Regimen

Newly diagnosed high-risk patients with neuroblastoma assigned to the first GPOH cohort of the study will start at cumulative dose level 1. Infusion duration of dinutuximab beta will be consistent throughout cumulative dose level 1 during the entire induction regimen. If toxicity is acceptable (estimated DLT probability <33%, see Section 6.7.3), then cumulative dose level 2 will be explored. The escalation is realized by increasing infusion duration 1 to infusion duration 2 in the second combination cycle. The rationale for design of this escalation pattern is an observed decrease in dinutuximab beta toxicity in subsequent cycles of dinutuximab beta therapy (13). Patients will receive the lower infusion duration in the first combination cycle followed by the higher infusion duration in the second combination cycle. In cumulative dose level 3, all infusion durations are increased to infusion duration 2 and stay consistent throughout the entire induction regimen.

After the evaluation of the DLTs to determine dinutuximab beta dose escalation or de-escalation during the first combination cycles, the infusion durations used in subsequent combination cycles will not be changed, except in the third N6 cycle of cumulative dose level 2 where infusion duration 2 was selected in order to keep a better balance for the total dose planned.

Dinutuximab beta de-escalation will follow the same strategy. De-escalation in cumulative dose level-1 is achieved by decreasing from infusion duration 1 to infusion duration-1 (for GPOH) in the first combination cycle but not the following combination cycle because of an improved toxicity profile of dinutuximab beta in subsequent cycles (13).

4.3.3.4 Rationale for the Dinutuximab Beta Dose-Finding Strategy in Combination with the COJEC Induction Chemotherapy Regimen

Newly diagnosed high-risk neuroblastoma patients assigned to the first COJEC cohort of the study will start in cumulative dose level 1. Dinutuximab beta infusion duration 1 will be consistent throughout cumulative dose level 1 during the entire induction regimen. If toxicity is acceptable (estimated DLT probability <330/, see Section 6.7.3), dose escalation in cumulative dose level 2 will be done by going from infusion duration 1 to infusion duration 2 in the second combination cycle. The rationale for design of this escalation pattern is an observed decrease in dinutuximab beta toxicity in subsequent cycles of dinutuximab beta therapy (13). Patients will receive the lower infusion duration in the first combination cycle followed by the higher infusion duration in the second combination cycle. In cumulative dose level 3, all infusion durations are increased to infusion duration 2 and stay consistent throughout the entire induction regimen.

Dinutuximab beta dose de-escalation will follow the same strategy. De-escalation in cumulative dose level −1 Is realized by decreasing from infusion duration 1 to infusion duration −1 (for COJEC) In the first 2 combination cycles but not in the third combination cycle because of an improved toxicity profile of dinutuximab beta in subsequent cycles (13).

After the evaluation of the toxicity to determine dinutuximab beta dose escalation or de-escalation during the first combination cycles, the infusion durations used in subsequent combination cycles are not changed.

### 4.4 End-of-Study Definition

A patient is considered to have completed the study treatment if he/she has completed all phases of the study including the last scheduled procedure shown in the Schedule of Activities (SoA) table (see Section 1.1).

The end of the study is defined as 100 days after the last patient has undergone standard-of-care HDC/ASCT or has started a new neuroblastoma treatment regimen instead of HDC/ASCT, whichever is earlier.

### 5.0 Study Population

Prospective approval of protocol deviations to inclusion and exclusion criteria, also known as protocol waivers or exemptions, is not permitted.

### 5.1 Inclusion Criteria

Patients are eligible for the study if they meet all the following inclusion criteria:
1. Established diagnosis of neuroblastoma Stage M, according to the INRGSS.
2. Age ≥18 months and <18 years.
3. Body weight: >12 kg.
4. Alanine transaminase (ALT) and aspartate aminotransferase (AST)<10×upper limit of normal (ULN), total bilirubin <1.5×ULN based on age specific reference ranges.
5. Creatinine clearance ≥70 mL/min/1.73 m² or creatinine <1.5×ULN corrected for age.
6. Shortening fraction ≥27% by echocardiogram or ejection fraction >50% by echocardiogram.
7. Able to comply with scheduled follow-up and with management of toxicity.
8. A female patient is eligible to participate if she is not pregnant, not breastfeeding, and at least 1 of the following conditions applies:
   i) Not a woman of childbearing potential (WOCBP).
      OR
   ii) A WOCBP who agrees to follow contraceptive guidance during the treatment period and for at least 1 year after the last dose of dinutuximab beta.
9. A male patient must agree to use contraception during the treatment period and for at least 1 year after the last dose of dinutuximab beta and refrain from donating sperm during this period.
10. Parent or guardian able to give consent, with the child giving assent when appropriate, which includes compliance with the requirements and restrictions listed in the informed consent form (ICF) and in this protocol.

### 5.2 Exclusion Criteria

Patients are excluded from the study if any of the following exclusion criteria apply:
1. Previous cancer-specific treatment for neuroblastoma.
2. Major surgery within 21 days prior to enrollment of the first dose of dinutuximab beta (open tumor biopsy or central line placement is not considered as major surgery).
3. History or evidence of severe acute or chronic infection or infectious illness requiring parenteral therapy unless fully healed at least 4 weeks prior to enrollment.
4. Patients with spinal cord involvement (symptomatic patients or if identified on imaging done to establish the diagnosis; there is no screening for spinal cord involvement for all patients).
5. Any other disease, metabolic or psychological dysfunction, physical examination finding, or clinical laboratory finding giving reasonable suspicion of a disease or condition that contraindicates use of an investigational drug or places the patient at unacceptable risk from treatment complications.

### 5.3 Lifestyle Considerations

No lifestyle restrictions are required for participation in this study.

### 5.4 Screen Failures

Screen failures are defined as patients who consent to participate in the clinical study but are not subsequently entered in the study. A minimal set of screen failure information is required to ensure transparent reporting of screen failure patients to meet the Consolidated Standards of Reporting Trials publishing requirements and to respond to queries from regulatory authorities. Minimal information Includes demography, screen failure details, eligibility criteria, and any serious AE (SAE).

Individuals who do not meet the criteria for participation in this study (screen failure) will not be rescreened.

### 6.0 Study Treatment

### 6.1 Study Treatment(s) Administration

### 6.1.1 Dinutuximab Beta

The study treatment under investigation in this study is the mouse-human chimeric monoclonal anti-GD2 immunoglobulin (Ig) G1 antibody dinutuximab beta, which will be provided to the center by the Sponsor as a liquid concentrate for the preparation of a solution for iv infusion.

The vials will be closed with fluorocarbon-coated halobutyl rubber stoppers and sealed with aluminum flip-off caps. The actual content of the respective batch of dinutuximab beta will be indicated on the label of the vials.

### 6.1.2 Dinutuximab Beta Administration

Dinutuximab beta will be diluted with 0.9% sodium chloride solution containing 1% human albumin at the site as described in the Pharmacy Manual. The individual dose of 10 mg/m²/day will be calculated based on the body surface area of the patient.

Dinutuximab beta must be administered according to the procedures described in this study protocol. Only patients enrolled in this study may receive study treatment. Only authorized site staff may supply or administer dinutuximab beta.

Continuous Infusions of dinutuximab beta will start at time points indicated for each of the chemotherapy cycles (Section 6.2). The start of dinutuximab beta in the morning is recommended for safety reasons. The total duration of the infusion will depend on the dinutuximab beta infusion duration planned for each patient and cycle. Each IV preparation will contain one, 24-hour dose (10 mg/m² dinutuximab beta) for the specific patient.

Dinutuximab beta will be delivered by continuous infusion using an electronic infusion pump at a flow rate of 2 mL/h±0.4 mL/h (see Section 6.7 for details of dinutuximab dose modifications). The IV preparation with dinutuximab beta will be replaced approximately every 24 hours.

If possible, the patient should receive the planned total dose of dinutuximab beta within each cycle. If the infusion is stopped or the flow rate decreased during the planned administration period, the planned flow rate cannot exceed 2 mL/h, but treatment duration may be extended by 20% of the planned time period in order to complete the dose per cycle as planned (a daily administration should be over 24 f 4.8 hours [20% error is allowed=20% rule] duration). Note, that adjustments in flow rate may be carried over to the next day's infusion as long as the maximum flow rate and duration for the day's infusion 28.8 hours) is not exceeded.

Chemical and physical in-use stability of dinutuximab beta has been demonstrated for up to 48 hours at 25° C. (50 mL syringe) and for up to 7 days at 37° C. (250 mL Infusion), after cumulative storage in a refrigerator (2° C. to 8° C.) for 72 hours. The solution for infusion must be prepared under aseptic conditions. Opened vials with remaining antibody solution must be discarded immediately. Preparation of dinutuximab beta for administration to the patients will be described in the Pharmacy Manual.

### Infusion Duration

Dose escalation and de-escalation will be accomplished by variation of the number of treatment days with dinutuximab beta at 10 mg/m²/day within a given chemotherapy cycle leading to different total infusion durations. Because each IV preparation will contain one, 24-hour dose (10 mg/m²), the number of IV preparation with dinutuximab beta corresponds to the number of treatment days.

### Cumulative Dose Level

The induction chemotherapy regimens consist of 5 (GPOH with dinutuximab beta) or 6 (COJEC with dinutuximab beta) cycles for the assessment of the combination.

The planned infusion durations are not (always) identical for all cycles of a given induction regimen. Variable infusion durations are used between the chemotherapy cycles of a given induction regimen. The reason for that variation is an expected increase in treatment tolerance for dinutuximab beta in subsequent treatment cycles.

The variation of infusion durations between cycles of a given induction chemotherapy regimen leads to different cumulative dose levels.

Dose escalation and de-escalation results from different cumulative dose levels planned for dinutuximab beta in the context of induction chemotherapy in the GPOH cohort (Table 10) and the COJEC cohort (Table 11) in combination with dinutuximab beta and each cumulative dose level is defined by varying infusion durations of dinutuximab beta within a chemotherapy cycle.

### 6.1.3 Chemotherapy

The chemotherapy drugs administered during the GPOH (cisplatin, etoposide, vindesine, ifosfamide, dacarbazine, vincristine, and doxorubicin) and COJEC (cisplatin, vincristine, carboplatin, etoposide, and cyclophosphamide) induction will be sourced by the study centers.

Details of the administration of chemotherapy will be described in the Pharmacy Manual, and the respective product labels should be referenced for additional information. The details of all treatments administered including, but not limited to, dose, dose interruptions, and dose modifications will be included in the CRF.

6.2 Composition of Chemotherapy Cycles Combined with Dinutuximab Beta

See FIG. 1 for details of the chemotherapy cycles and Section 6.7 for details of dinutuximab beta dose modification guidance.

### General Comment:

Given number of medications given simultaneously including hydration, chemotherapy, dinutuximab beta, and supportive care as well as the need for PK sampling, it is recommended that patients have a double-lumen central line to facilitate their care.
Antiemetic drugs will be used according to institutional standards. Due to the immunosuppressive activity, corticosteroids are not permitted to be used.
The cardio protectant dexrazoxane should not be administered.
   6.2.1 Dinutuximab Beta and GPOH induction Chemotherapy

The Induction treatment will be applied over 18 weeks in 21-day cycles. Two different courses will be given every 21 days.

The GPOH induction chemotherapy regimen is administered in 21-day cycles depending in particular on hematologic recovery and on recovery from other organ toxicities.

The first cycle with N5 will be without dinutuximab beta. The remainder of the cycles will be in combination with dinutuximab beta.

6.2.1.1 Cycle N5 without Dinutuximab Beta

| Criteria for the Start of N5 | Absolute neutrophil count (ANC) ≥500/μL (except | | | | | | | | | | | | |
|---|---|---|---|---|---|---|---|---|---|---|---|---|---|
| | patients with extensive bone marrow involvement). | | | | | | | | | | | | |
| | Platelets ≥50000/μL without platelet transfusions | | | | | | | | | | | | |
| | (except patients with extensive bone marrow involvement). | | | | | | | | | | | | |
| | Creatinine clearance and/or cystatin C clearance | | | | | | | | | | | | |
| | ≥60 mL/min × 1.73 m² corresponding to National Cancer | | | | | | | | | | | | |
| | Institute (NCI) Common Terminology Criteria for | | | | | | | | | | | | |
| | AES (CTCAE) version 5.0 Grade ≥2 toxicity. | | | | | | | | | | | | |
| | No active infection. | | | | | | | | | | | | |
| Treatment Day | 1 | 2 | 3 | 4 | 5 | 6 | 7 | 8 | 9 | 10 | 11 | 12 | . . . |
| Cisplatin, 40 mg/m²/d iv, ctn | x | x | x | x | | | | | | | | | |
| (96 h) | | | | | | | | | | | | | |
| Etoposide, 100 mg/m²/d iv ctn | x | x | x | x | | | | | | | | | |
| (96 h) | | | | | | | | | | | | | |
| Vindesine, 3 mg/m²/d iv max. | x | | | | | | | | | | | | |
| 6 mg (1 h) | | | | | | | | | | | | | |
| Hydration 3000 mL/m²/d, with | x | x | x | x | x | x | | | | | | | |
| Mg, Ca, Kª (144 h) | | | | | | | | | | | | | |
| G-CSF, 5 μg/kg/d sc | | | | | | | | | x | x | x | x | x |

Abbreviations: ctn = continuous; G-CSF = granulocyte-colony stimulating factor;
iv = intravenous; max = maximum.
^{a}recommended fluid: 3000 mL/m²/24 h composed of 0.45% saline, 2.5% dextrose,
20 mmol/L KCl, 3.3 mmol/L MgSO₄ and 0.83 mmol/L calcium gluconate.

| Timing Within Cycle | Hydration for a total of 144 hours with 3000 | | |
|---|---|---|---|
| | mL/m²/d composed of 0.45% saline, 2.5% | | |
| | dextrose, 20 mmol/L KCl, 3.3 mmol/L MgSO₄ and | | |
| | 0.83 mmol/L calcium gluconate. Magnesium is | | |
| | given for renal protection (14 to 17). | | |
| Dose Adaptions for | Cisplatin | 1.3 mg/kg/d iv | ctn (96 h) |
| Children ≥12 kg | Etoposide | 4.2 mg/kg/d iv | ctn (96 h) |
| | Vindesine | 0.1 mg/kg/d iv | (1 h) |

| | Abbreviations: ctn = continuous; iv = intravenous. |
|---|---|
| Dose Modifications | |
| Hearing Impairment | Substitution of cisplatin by carboplatin |
| Grade ≥3 | 160 mg/m²/d (infants 5.3 mg/kg/d) as a |
| | continuous infusion over 96 hours. |
| Renal Function | If glomerular filtration rate (GFR) |
| | ≤60 mL/min/m², then substitute cisplatin with |
| | carboplatin (160 mg/m²/day for 4 days as a |
| | continuous infusion over 96 hours). Full-dose |
| | etoposide. |
| | Any other unresolved Grade ≥3 toxicity needs to |
| | be discussed with the Sponsor or contract |
| | research organization (CRO) Medical Monitor. |

6.2.1.2 Cycle N6 with Dinutuximab Beta

| Criteria for the Start of N6 | Absolute neutrophil count (ANC) ≥500/μL (except | | | | | | | | | | | | |
|---|---|---|---|---|---|---|---|---|---|---|---|---|---|
| | patients with extensive bone marrow involvement). | | | | | | | | | | | | |
| | Platelets ≥50000/μL without platelet transfusion | | | | | | | | | | | | |
| | (except patients with extensive bone marrow involvement). | | | | | | | | | | | | |
| | No cardiomyopathy NCI-CTCAE version 5.0 Grade | | | | | | | | | | | | |
| | ≥3 (Resting ejection fraction 39% to 20%; >20% | | | | | | | | | | | | |
| | drop from baseline), echocardiography prior to each | | | | | | | | | | | | |
| | N6 cycle required. | | | | | | | | | | | | |
| | Creatinine clearance and/or cystatin C clearance | | | | | | | | | | | | |
| | ≥60 mL/min × 1.73 m², corresponding to NCI-CTCAE | | | | | | | | | | | | |
| | version 5.0 Grade ≥2 toxicity. | | | | | | | | | | | | |
| | No active infection. | | | | | | | | | | | | |
| Treatment Day | 1 | 2 | 3 | 4 | 5 | 6 | 7 | 8 | 9 | 10 | 11 | 12 | . . . |
| Ifosfamide, 1500 mg/m²/d iv ctn | x | x | x | x | x | | | | | | | | |
| (115 h) | | | | | | | | | | | | | |
| Dacarbazine, 200 mg/m²/d iv, | x | x | x | x | x | | | | | | | | |
| (1 h) | | | | | | | | | | | | | |
| Vincristine, 1.5 mg/m²/d iv max | x | | | | | | | x | | | | | |
| 2 mg (1 h) | | | | | | | | | | | | | |
| Doxorubicin, 30 mg/m²/d iv (4 h) | | | | | | x | x | | | | | | |
| Hydration 3000 mL/m²/d with | x | x | x | x | x | x | x | | | | | | |
| MESNA 900 mg/m²/d (168 h)^{a} | | | | | | | | | | | | | |
| Hydration 1500 mL/m²/d iv/po | | | | | | | | x^{b} | | | | | |
| until end of dinutuximab | | | | | | | | | | | | | |
| G-CSF, 5 μg/kg/d sc | | | | | | | | | | x | x | x | x |
| Dinutuximab beta | | | | | | | | | | | | | |
| (see also Table 10) | | | | | | | | | | | | | |
| Infusion duration −1: | | | | x | x | x | | | | | | | |
| 10 mg/m²/d ctn (72 h) | | | | | | | | | | | | | |
| Infusion duration 1: | | | | x | x | x | x | x | | | | | |
| 10 mg/m²/d ctn (120 h) | | | | | | | | | | | | | |
| Infusion duration 2: | | x | x | x | x | x | x | x | | | | | |
| 10 mg/m²/d ctn (168 h) | | | | | | | | | | | | | |

Abbreviations: ctn = continuous; G-CSF = granulocyte-colony stimulating factor; iv = intravenous; max = maximum; po = orally; sc = subcutaneous.
^{a}recommended composition of 1000 mL hydration fluid: 3 mL MESNA 100 mg/mL, 485 mL NaCl 0.9%, 485 mL glucose 5%, 25 mL KCl 7.45%,
^{b}Apply only when dinutuximab beta is given.

| Timing within Cycle | Hydration for a total of 168 hours with 3000 | | |
|---|---|---|---|
| | mL/m²/d of 0.45% saline containing 2.5% | | |
| | dextrose, and 30 mL/3000 mL KCl 7.46%. | | |
| | Parallel infusion of MESNA 900 mg/m²/d as a | | |
| | continuous 24-hour infusion on Days 1 to 7, ie, | | |
| | 168 hours. MESNA can be added to the hydration | | |
| | fluid. No extra line needed. | | |
| | Ifosfamide infusion must be paused during | | |
| | dacarbazine infusion in order to avoid interactions | | |
| | between both drugs. | | |
| | Start of gabapentin will be variable and depend on | | |
| | the DL of dinutuximab beta. | | |
| | gabapentin will start 3 days before the first dose of | | |
| | dinutuximab beta (Day −3). | | |
| | Dosing of gabapentin: | | |
| | 10 mg/kg/dose po. | | |
| | Day −3: 1 × 10 mg/kg po; Day −2: 2 × 10 mg/kg | | |
| | po; Day −1: 3 × 10 mg/kg po. | | |
| | Gabapentin will be tapered on the first day after the | | |
| | end of dinutuximab beta infusion (Day +1). | | |
| | Day +1: 2 × 10 mg/kg po; Day +2: 1 × 10 mg/kg | | |
| | po; Day +3: stop. | | |
| | Morphine continuous infusion (30 μg/kg/h) will start | | |
| | 1 hour before dinutuximab beta infusion. | | |
| | Morphine continuous infusion can | | |
| | be tapered | | |
| | starting from Day 2 of dinutuximab beta infusion. | | |
| | Antipyretic drug continuous co-medication and | | |
| | other supportive care measures for dinutuximab | | |
| | beta will start 1 hour before dinutuximab beta | | |
| | infusion. | | |
| | Refer to Section 6.6. | | |
| | Dinutuximab beta infusion will start at 2 mL/h (for | | |
| | Details refer to the Section 6.1). | | |
| Dose Adaptations for | Ifosfamide | 50 mg/kg/d iv | ctn (115 h) |
| Children ≤12 kg | Dacarbazine | 6.7 mg/kg/d iv | (1 h) |
| | Vincristine | 0.05 mg/kg/d iv | (1 h) |
| | Doxorubicin | 1 mg/kg/d iv | (4 h) |
| | Hydration | 3000 mL/m²/d with | ctn |
| | | MESNA 30 mg/kg/d | |

| | |
|---|---|
| | Abbreviations: ctn = continuous; iv = intravenous. |
| Dose Modifications | |
| Delayed Bone Marrow | First delay: reduction of ifosfamide to 1000 |
| Recovery to an Interval | mg/m²/d (33 mg/kg/day for children ≤12 kg). Full- |
| of >28 days to the First Day | dose for other drugs. |
| of Subsequent Cycle OR | Second delay: omit dacarbazine in the next N6 cycle |
| Grade 4 Infection | and contact the Sponsor or CRO Medical Monitor. |
| | Full-dose for other drugs. |
| Renal Function | If GFR ≤60 mL/min/m² then substitute ifosfamide |
| | with cyclophosphamide 300 mg/m²/d continuous |
| | infusion Days 1 to 5 and discuss with the Sponsor |
| | or CRO Medical Monitor. |
| Ifosfamide Induced | Application of methylene blue (according to the |
| Encephalopathy | institutional policy) as clinically indicated. Notify the |
| NCI-CTCAE version 5.0 | Sponsor or CRO Medical Monitor if substitution of |
| Grade ≥3 | ifosfamide by cyclophosphamide 300 mg/m²/d |
| | continuous infusion Days 1 to 5. |
| Any | To be discussed with the Sponsor or CRO Medical |
| other Unresolved | Monitor. |
| Grade ≥3 Toxicity | |

6.2.1.3 Cycle N5 with Dinutuximab Beta

| Criteria for the Start of N5 | ANC ≥500/μL without G-CSF for at least 48 hours. | | | | | | | | | | | | |
|---|---|---|---|---|---|---|---|---|---|---|---|---|---|
| | Platelets ≥50000/μL without platelet transfusions | | | | | | | | | | | | |
| | (except patients with extensive bone marrow involvement). | | | | | | | | | | | | |
| | No ototoxicity NCI-CTCAE version 5.0 Grade ≥3, | | | | | | | | | | | | |
| | audiometry prior to each N5 cycle required when | | | | | | | | | | | | |
| | cisplatin is given. | | | | | | | | | | | | |
| | Creatinine clearance and/or cystatin C clearance | | | | | | | | | | | | |
| | ≥60 ml/min × 1.73 m² corresponding to NCI-CTCAE | | | | | | | | | | | | |
| | version 5.0 Grade ≥2 toxicity. | | | | | | | | | | | | |
| | No active infection. | | | | | | | | | | | | |
| Treatment Day | 1 | 2 | 3 | 4 | 5 | 6 | 7 | 8 | 9 | 10 | 11 | 12 | . . . |
| Cisplatin, 40 mg/m²/d iv, ctn (96 h) | x | x | x | x | | | | | | | | | |
| Etoposide, 100 mg/m²/d iv ctn | x | x | x | x | | | | | | | | | |
| (96 h) | | | | | | | | | | | | | |
| Vindesine, 3 mg/m²/d iv max. 6 mg | x | | | | | | | | | | | | |
| (1 h) | | | | | | | | | | | | | |
| Hydration 3000 mL/m²/d, with Mg, | x | x | x | x | x | x | | | | | | | |
| Ca, Ka (144 h) | | | | | | | | | | | | | |
| Hydration 1500 mL/m²/d iv/po until | | | | | | | x^{b} | x^{b} | | | | | |
| end of dinutuximab | | | | | | | | | | | | | |
| G-CSF, 5 μg/kg/d sc | | | | | | | | | x | x | x | x | x |
| Dinutuximab beta | | | | | | | | | | | | | |
| (see also Table 10) | | | | | | | | | | | | | |
| Infusion duration −1: | | | | x | x | x | | | | | | | |
| 10 mg/m²/d ctn (72 h) | | | | | | | | | | | | | |
| Infusion duration 1: | | | | x | x | x | x | x | | | | | |
| 10 mg/m²/d ctn (120 h) | | | | | | | | | | | | | |
| Infusion duration 2 | | x | x | x | x | x | x | x | | | | | |
| 10 mg/m²/d ctn (168 h) | | | | | | | | | | | | | |

Abbreviations: ctn = continuous; G-CSF = granulocyte-colony stimulating factor; iv = intravenous; max = maximum; po = orally; sc = subcutaneous.
^{a}recommended composition of 1000 mL hydration fluid: composed of 0.45% saline, 2.5% dextrose, 20 mmol/L KCl, 3.3 mmol/L MgSO₄ and 0.83 mmol/L calcium gluconate.
^{b}apply only when dinutuximab beta is given.

| Timing Within Cycle | Hydration for a total of 144 hours with 3000 mL/m²/d | | |
|---|---|---|---|
| | composed of 0.45% saline, 2.5% dextrose, | | |
| | 20 mmol/L KCl, 3.3 mmol/L MgSO₄ and | | |
| | 0.83 mmol/L calcium gluconate. Magnesium is given | | |
| | for renal protection (14 to 17). | | |
| | Start of gabapentin will be variable and depend on the | | |
| | DL of dinutuximab beta. | | |
| | Gabapentin administration will start 3 days before the | | |
| | first dose of dinutuximab beta (Day −3). | | |
| | Dosing of gabapentin: 10 mg/kg/dose po. | | |
| | Day −3: 1 × 10 mg/kg po; Day −2: 2 × 10 mg/kg po; | | |
| | Day −1 3 × 10 mg/kg po. | | |
| | Gabapentin will be tapered on the first day after the end | | |
| | of dinutuximab beta infusion (Day +1). | | |
| | Day +1: 2 × 10 mg/kg po; Day +2, 1 × 10 mg/kg po; | | |
| | Day +3 stop. | | |
| | Morphine continuous infusion (30 μg/kg/h) will start | | |
| | 1 hour before dinutuximab beta infusion. | | |
| | Morphine continuous infusion can be tapered starting | | |
| | from Day 2 of dinutuximab beta infusion. | | |
| | Antipyretic drug continuous co-medication and other | | |
| | supportive care measures for dinutuximab beta will | | |
| | start not later than 1 hour before dinutuximab beta | | |
| | infusion. | | |
| | Refer to Section 6.6. | | |
| | Dinutuximab beta infusion will start at 2 mL/hour (for | | |
| | details, refer to Section 6.1). | | |
| Dose Adaptations for | Cisplatin | 1.3 mg/kg/d iv | ctn (96 h) |
| Children ≤12 kg | Etoposide | 4.2 mg/kg/d iv | ctn (96 h) |
| | Vindesine | 0.1 mg/kg/d iv | (1 h) |

| | |
|---|---|
| | Abbreviations: ctn = continuous; iv = intravenous. |
| Dose Modifications | |
| Delayed Bone Marrow | Reduction of etoposide to 3.2 mg/kg/d in infants and |
| Recovery to an Interval | 80 mg/m²/d in children >1 year in the next N5 cycle is |
| of >28 days to the First Day | recommended. Of note, infant etoposide doses are |
| of Subsequent Cycle or | derived from plasma level and are higher than in |
| Grade 4 Infection | children >1-year-old. Full-dose of cisplatin. |
| Ototoxicity Grade ≥3 | Substitution of cisplatin by carboplatin 160 mg/m²/d |
| | (infants 5.3 mg/kg/d) as a continuous infusion over |
| | 96 hours. |
| Renal Function | If GFR ≤60 mL/min/m² then substitute cisplatin with |
| | carboplatin (160 mg/m²/day for 4 days as continuous |
| | infusion over 96 hours). Full-dose etoposide. |
| Any Other Unresolved | To be discussed with the Sponsor or the CRO Medical |
| Grade ≥3 Toxicity | Monitor. |

### 6.2.2 Dinutuximab Beta and COJEC Induction Chemotherapy

The Induction treatment will be applied over 10 weeks in 10-day cycles and proceed regardless of neutrophil or platelet counts and controlled infection. Three different courses will be given every 10 days. Consult the Sponsor or CRO Medical Monitor If GFR is <80 mL/min×1.73 m².

The COJEC Induction chemotherapy regimen is administered in 10-day cycles.

The use of G-CSF 5 μg/kg/day sc during induction Cycle B is recommended starting 24 hours after the last chemotherapy and stopped the day prior to commencing the next course with an interval of at least 24 hours between the last G-CSF injection and the start of chemotherapy.

Use of polyethylene glycol-filgrastim will not be permitted.

The first 2 cycles, consisting of Cycle A and Cycle B, will be without dinutuximab beta. The remainder of cycles will be in combination with dinutuximab beta.

The First Cycles A and B are without Dinutuximab Beta.
6.2.2.1 Cycle A without Dinutuximab Beta

| Start | Day 1 | | | | | | | |
|---|---|---|---|---|---|---|---|---|
| Treatment Day | 1 | 2 | 3 | 4 | 5 | 6 | 7 | 8 |
| Vincristine, 1.5 mg/m²/d iv max 2 mg (bolus) | x | | | | | | | |
| Carboplatin, 750 mg/m²/d iv (1 h) | x | | | | | | | |
| Etoposide, 175 mg/m²/d iv, (4 h) | x | x | | | | | | |
| Hydration 2000 mL/m²/d iv | x | | | | | | | |
| G-CSF, 5 μg/kg/d sc | | | x | x | x | x | x | x |

Abbreviations: G-CSF = granulocyte-colony stimulating factor; iv = intravenous;
max = maximum; po = orally; sc = subcutaneous.

| Timing Within Cycle A | Vincristine 0 h 1.5 mg/m² (maximum dose 2 mg) as a | | |
|---|---|---|---|
| | single iv bolus. | | |
| | Carboplatin 0 h 750 mg/m² standard size preparaations | | |
| | infused over 1 hour in 5% dextrose. | | |
| | Etoposide 1 h 175 mg/m² standard size preparations | | |
| | infused over 4 hours in 0.9% saline. | | |
| | Chemotherapy of Cycle A will start with vincristine | | |
| | bolus and immediate start of the carboplatin infusion | | |
| | (1 hour) followed immediately by etoposide infusion | | |
| | (4 hours). | | |
| Dose Adaptations for | Vincristine | 0.05 mg/kg/d iv | (bolus) |
| Children ≤12 Kg | Carboplatin | 25 mg/kg/d iv | (1 h) |
| | Etoposide | 5.833 mg/kg/d iv | (4 h) |
| | Abbreviation: iv = intravenous. | | |
| Dose Modifications | | | |

| | |
|---|---|
| Infection: | Grade 4: reduce carboplatin and etoposide doses to |
| | 75% of full-dose. Administer full-dose vincristine. |
| Hepatotoxicity | Do not alter doses for abnormal transaminases. If total |
| | bilirubin 26 to 50 μmol/L, then give 50% dose |
| | etoposide, if total bilirubin ≥51 μmol/L, omit etoposide |
| | and vincristine. Resume normal doses in subsequent |
| | cycle once liver function has improved. No dose |
| | modifications to carboplatin are required. Resume |
| | normal doses in subsequent cycle once liver function |
| | has improved. |
| Neurotoxicity | Omit vincristine if severe constipation NCI-CTCAE |
| | version 5.0 Grade 23 or severe foot drop/ptosis. |
| | Resume at 66% dose in subsequent cycle if recovered. |
| Renal Function | No modifications are required if urine output is normal. |
| Any Other Unresolved | To be discussed with the Sponsor or the CRO Medical |
| Grade ≥3 Toxicity | Monitor. |

6.2.2.2 Cycle B without Dinutuximab Beta

| Start | Day 11 | | | | | | | |
|---|---|---|---|---|---|---|---|---|
| Treatment Day | 1 | 2 | 3 | 4 | 5 | 6 | 7 | 8 |
| Vincristine, 1.5 mg/m²/d iv max 2 mg (bolus) | x | | | | | | | |
| Cisplatin, 80 mg/m²/d iv ctn (24 h) | x | | | | | | | |
| Hydration 3000 mL/m²/d (48 h) iv | x | x | | | | | | |
| G-CSF, 5 μg/kg/d sc | | x | x | x | x | x | x | x |

Abbreviations: ctn = continuous; G-CSF = granulocyte-colony stimulating factor;
iv = intravenous; max = maximum; sc = subcutaneous.

| | |
|---|---|
| Timing Within Cycle B | Vincristine iv bolus or 1-hour infusion according to |
| | local policies. |
| | Prehydration for cisplatin: Infuse at 200 mL/m²/h |
| | for 3 hours 0.9% sodium chloride with 10 mmol/L |
| | potassium chloride. |
| | Mannitol 20% at 0 h short infusion at a dose of |
| | 40 ml/m². |
| | Mannitol 20% at 2.5 h short infusion at a dose of |
| | 40 mL/m². |
| | Cisplatin at 3 h 80 mg/m² over 24 hours in a mini- |
| | bag alongside the hydration for 24 hours in 0.9% |
| | sodium chloride. |
| | Commence hydration in parallel with cisplatin: |
| | 1.5 L/m²/24 h of 0.9% sodium chloride, |
| | 1.5 L/m²/24 h of 5% glucose, 30 mmol/m²/24 h of |
| | potassium chloride, 2.5 mmol/m²/24 h of calcium |
| | gluconate 10 mmol/m²/24 h of magnesium sulfate. |
| | Mannitol 20% at 9 h if urine output falls below |
| | 400 mL/m²/6 h, short infusion at a dose of 40 |
| | mL/m² repeat thereafter whenever indicated. |
| | 27 h commence post-cisplatin hydration: |
| | 1.5 L/m²/24 h of 0.9% sodium chloride, |
| | 1.5 L/m²/24 h of 5% glucose, 60 mmol/m²/24 h of |
| | potassium chloride, 2.5 mmol/m² calcium |
| | gluconate, 10 mmol/m²/24 h of magnesium sulfate. |

During prehydration, the cisplatin infusion together with its parallel hydration and post-cisplatin hydration,
a careful record of fluid input and output should be kept to prevent hydration overload and ensure diuresis.
Cisplatin is given as a continuous infusion over 24 hours combined with forced mannitol diuresis: Mannitol
20% at a dose of 40 mL/m² should be administered 3 hours and 30 minutes prior to starting cisplatin and
thereafter if urine output falls below 400 mL/m²/6 h (during the cisplatin infusion furosemide should be
avoided because of its enhancing effect on ototoxicity). The addition of magnesium during cisplatin
treatment is recommended at a daily dose of 180 mg/m²/day during the induction period but may need
to be adjusted following strict monitoring of magnesium levels. Mannitol and magnesium are not to be
given concurrently, as mannitol and magnesium are not compatible. The addition of calcium, potassium,
and phosphate may also be modified according to serum levels.

| | | | |
|---|---|---|---|
| Dose Adaptations for | Vincristine | 0.05 mg/kg/d iv max 2 mg | (bolus) |
| Children ≤12 kg | Cisplatin | 2.666 mg/kg/d iv ctn | (24 h) |

| | Abbreviations: ctn = continuous; iv = intravenous; max = maximum. |
|---|---|
| Dose Modifications | |
| Infection | Grade 4: no modifications |
| Hepatotoxicity | Do not alter doses for abnormal transaminases. If |
| | total bilirubin ≥51 μmol/L, omit vincristine. Resume |
| | normal doses in subsequent cycle once liver |
| | function has improved. No dose modifications to |
| | cisplatin required. Resume normal doses in |
| | subsequent cycle once liver function has improved. |
| Neurotoxicity | Omit vincristine if severe constipation Grade ≥3 or |
| | severe foot drop/ptosis. Resume at 66% dose in |
| | subsequent cycle if recovered. |
| Renal Function | If GFR ≤60 ml/min/m² then contact Sponsor or |
| | CRO Medical Monitor. |
| Ototoxicity | Grade ≥3 then omit cisplatin and contact Sponsor |
| | or CRO Medical Monitor. |
| Any Other Unresolved | To be discussed with the Sponsor or the CRO |
| Grade ≥3 Toxicity | Medical Monitor. |

6.2.2.3 Cycle with Dinutuximab Beta

| Start | Days 21 and 61 | | | | | | | |
|---|---|---|---|---|---|---|---|---|
| Treatment Day | 1 | 2 | 3 | 4 | 5 | 6 | 7 | 8 |
| Vincristine, 1.5 mg/m²/d iv max 2 mg (1 h) | x | | | | | | | |
| Cyclophosphamide, 1.05 g/m²/d iv (1 h) | x | x | | | | | | |
| Etoposide, 175 mg/m²/d iv, (4 h) | x | x | | | | | | |
| Hydration 3000 mL/m²/d iv | x | x | | | | | | |
| Hydration 1500 mL/m²/d iv/po | | | x | xª | | | | |
| G-CSF, 5 μg/kg/d sc | | | x | x | x | x | x | x |
| Dinutuximab beta (see also Table 11) | | | | | | | | |
| Infusion duration −1: | | x | x | | | | | |
| 10 mg/m²/d ctn (48 h) | | | | | | | | |
| Infusion duration 1: | | x | x | x | | | | |
| 10 mg/m²/d ctn (72 h) | | | | | | | | |
| Infusion duration 2: | x | x | x | x | | | | |
| 10 mg/m²/d ctn (96 h) | | | | | | | | |

Abbreviations: ctn = continuous; G-CSF = granulocyte-colony stimulating factor;
iv = intravenous; max = maximum; po = oral; sc = subcutaneous.
^{a}apply only when dinutuximab beta is given.
During post-cyclophosphamide infusion a careful record of fluid input and output should
be kept to prevent fluid overload and ensure an adequate diuresis. If urine output falls
less than 400 mL/m²/6 h, furosemide 0.5 to 1.0 mg/kg should be given.

| Timing Within Cycle C | Vincristine at 0 h 1.5 mg/m² (maximum dose 2 mg) | | |
|---|---|---|---|
| | iv bolus or 1-hour infusion according to local | | |
| | policies. | | |
| | Etoposide at 0 h 175 mg/m² standard size bags | | |
| | infused over 4 hours in 0.9% saline. | | |
| | MESNA bolus at 4 h 200 mg/m² iv bolus. | | |
| | Cyclophosphamide at 4 h 1.05 gm/m² as iv bolus | | |
| | followed by post-cyclophosphamide infusion for | | |
| | 24 hours with 1.2 g/m²/24 h MESNA, 1.5 L/m²/24 h | | |
| | of 0.9% sodium chloride, 1.5 L/m²/24 h 5% | | |
| | glucose, 60 mmol/m²/24 h of potassium chloride. | | |
| | Etoposide at 28 h 175 mg/m² standard size bags | | |
| | infused over 4 hours as above. | | |
| | Cyclophosphamide 32 h 1.05 gm/m² as iv bolus | | |
| | followed by post-cyclophosphamide infusion for | | |
| | 24 hours with: 1.2 g/m²/24 h MESNA, 1.5 L/m²/24 h | | |
| | of 0.9% sodium chloride, 1.5 L/m²/24 h 5% | | |
| | glucose, 60 mmol/m²/24 h of potassium chloride. | | |
| | Start of gabapentin is variable and depends on the | | |
| | DL of dinutuximab beta. | | |
| | Gabapentin will start 3 days before the first dose of | | |
| | dinutuximab beta (Day −3). | | |
| | Dosing of gabapentin: 10 mg/kg/dose po. | | |
| | Day −3: 1 × 10 mg/kg po; Day −2: 2 × 10 mg/kg | | |
| | po; Day −1: 3 × 10 mg/kg po. | | |
| | Gabapentin is tapered on first day after end of | | |
| | dinutuximab beta infusion (Day +1). | | |
| | Day +1: 2 × 10 mg/kg po; Day +2: 1 × 10 mg/kg | | |
| | po; Day +3: stop. | | |
| | Morphine continuous infusion (30 μg/kg/h) starts | | |
| | 1 hour before dinutuximab beta infusion. | | |
| | Morphine ctn infusion can be tapered starting from | | |
| | Day 2 of dinutuximab beta infusion. | | |
| | Antipyretic drug continuous co-medication and | | |
| | other supportive care measures for dinutuximab | | |
| | beta starts 1 hour before dinutuximab beta infusion. | | |
| | Refer to Appendix 3. | | |
| | Dinutuximab beta infusion will start at 2 mL/h (for | | |
| | Details refer to the Section 6.1). | | |
| Dose Adaptations for | Vincristine | 0.05 mg/kg/d iv max 2 mg | (1 h) |
| Children ≤12 kg | Cyclophosphamide | 35 mg/kg/d iv | (1 h) |
| | Etoposide | 5.833 mg/kg/d iv | (4 h) |

| | Abbreviations: iv = intravenous; max = maximum. |
|---|---|
| Dose Modifications | |
| Infection | Grade 4: reduce cyclophosphamide and etoposide |
| | doses to 75% of full-dose. Administer full-dose |
| | vincristine. |
| Hepatotoxicity | Do not alter doses for abnormal transaminases. If |
| | total bilirubin 26 to 50 μmol/L, then give 50% dose |
| | etoposide; if total bilirubin ≥51 μmol/L, then omit |
| | etoposide and vincristine. Resume normal doses in |
| | subsequent cycle once liver function has improved. |
| | No dose modifications to cyclophosphamide |
| | required. Resume normal doses in subsequent cycle |
| | once liver function has improved. |
| Neurotoxicity | Omit vincristine if severe constipation Grade ≥3 or |
| | severe foot drop/ptosis. Resume at 66% dose in |
| | subsequent cycle if recovered. |
| Renal Function | No modifications required if normal urine output. |
| Hemorrhagic Cystitis in | Increase MESNA dose by 50% and increase |
| Previous Cycle | hydration per institutional standards. |
| Any Other Unresolved | To be discussed with the Sponsor or the CRO |
| Grade 23 Toxicity | Medical Monitor. |

6.2.2.4 Cycle B with Dinutuximab Beta

| Start | Days 31, 51 and 71. | | | | | | | |
|---|---|---|---|---|---|---|---|---|
| Treatment Day | 1 | 2 | 3 | 4 | 5 | 6 | 7 | 8 |
| Vincristine, 1.5 mg/m²/d iv max 2 mg (bolus) | x | | | | | | | |
| Cisplatin, 80 mg/m²/d iv ctn (24 h) | x | | | | | | | |
| Hydration 3000 mL/m²/d (48 h) iv | x | x | | | | | | |
| Hydration 1500 mL/m²/d iv/po | | | x | | | | | |
| G-CSF, 5 μg/kg/d sc | | x | x | x | x | x | x | x |
| Dinutuximab beta (see also Table 11) | | | | | | | | |
| Infusion duration −1: | | x | x | | | | | |
| 10 mg/m²/d ctn (48 h) | | | | | | | | |
| Infusion duration | | x | x | x | | | | |
| 10 mg/m²/d ctn (72 h) | | | | | | | | |
| Infusion duration 2 | x | x | x | x | | | | |
| 10 mg/m²/d ctn (96 h) | | | | | | | | |

Abbreviations: ctn = continuous; G-CSF = granulocyte-colony stimulating factor;
iv = intravenous; max = maximum; po = oral; sc = subcutaneous.
^{a}apply only when dinutuximab beta is given

| | |
|---|---|
| Timing Within Cycle B | Vincristine iv bolus or 1-hour infusion according to local |
| | policies. |
| | Prehydration for cisplatin: infuse at 200 ml/m²/h for |
| | 3 hours 0.9% sodium chloride with 10 mmol/L |
| | potassium chloride. |
| | Mannitol 20% at 0 h short infusion at a dose of |
| | 40 mL/m². |
| | Mannitol 20% at 2.5 h short infusion at a dose of |
| | 40 mL/m². |
| | Cisplatin at 3 h 80 mg/m² over 24 hours in a mini-bag |
| | alongside the hydration for 24 hours in 0.9% sodium |
| | chloride. |
| | Commence hydration in parallel with cisplatin: |
| | 1.5 L/m²/24 h of 0.9% sodium chloride, 1.5 L/m²/24 h |
| | of 5% glucose, 30 mmol/m²/24 h of potassium |
| | chloride, 2.5 mmol/m²/24 h of calcium gluconate |
| | 10 mmol/m²/24 h of magnesium sulfate. |
| | Mannitol 20% at 9 h if diuresis falls below |
| | 400 mL/m²/6 h, short infusion at a dose of 40 mL/m² |
| | repeat thereafter whenever indicated. |
| | Commence post-cisplatin hydration at 27 h: |
| | 1.5 L/m²/24 h of 0.9% sodium chloride, 1.5 l/m²/24 h |
| | of 5% glucose, 60 mmol/m²/24 h of potassium |
| | chloride, 2.5 mmol/m2 calcium gluconate, |
| | 10 mmol/m²/24 h of magnesium sulfate. |
| | Start of gabapentin is variable and depends on the DL |
| | of dinutuximab beta. |
| | Gabapentin will start 3 days before the first dose of |
| | dinutuximab beta (Day −3). |
| | Dosing of gabapentin: 10 mg/kg/dose po. |
| | Day −3: 1 × 10 mg/kg po; Day −2: 2 × 10 mg/kg po; |
| | Day −1: 3 × 10 mg/kg po. |
| | Gabapentin is tapered on first day after end of |
| | dinutuximab beta infusion (Day +1). |
| | Day +1: 2 × 10 mg/kg po; Day +2: 1 × 10 mg/kg po; |
| | Day +3: stop. |
| | Morphine continuous infusion (30 μg/kg/h) starts 1 |
| | hour before dinutuximab beta infusion. |
| | Morphine continuous infusion can be tapered starting |
| | from Day 2 of dinutuximab beta infusion. |
| | Antipyretic drug continuous co-medication and other |
| | supportive care measures for dinutuximab beta starts |
| | 1 hour before dinutuximab beta infusion. Refer to |
| | Appendix 3. |
| | Dinutuximab beta infusion starts at 2 mL/h (for Details |
| | refer to the Section 6.1). |

During prehydration, the cisplatin infusion together with its parallel hydration and post-cisplatin hydration,
a careful record of fluid input and output should be kept to prevent hydration overload and ensure diuresis.
Cisplatin is given as a continuous infusion over 24 hours combined with forced mannitol diuresis: Mannitol
20% at a dose of 40 ml/m² should be administered 3 hours and 30 minutes prior to starting cisplatin and
thereafter if urine output falls below 400 mL/m²/6 h (during the cisplatin infusion furosemide should be
avoided because of its enhancing effect on ototoxicity). The addition of magnesium during cisplatin treatment
is recommended at a daily dose of 180 mg/m²/day during the induction period but may need to be adjusted
following strict monitoring of magnesium levels. Mannitol and magnesium are not to be given concurrently
as mannitol and magnesium are not compatible. The addition of calcium, potassium, and phosphate may also
be modified according to serum levels.

| Dose Adaptations for | Vincristine | 0.05 mg/kg/d iv max 2 mg | (bolus) |
|---|---|---|---|
| Children ≤12 kg | Cisplatin | 2.666 mg/kg/d iv ctn | (24 h) |
| | Abbreviations: ctn = continuous; iv = intravenous; max = maximum. | | |
| Dose Modifications | | | |

| | |
|---|---|
| Infection | Grade 4: no modifications. |
| Hepatotoxicity | Do not alter doses for abnormal transaminases. If total |
| | bilirubin ≥51 umol/L omit vincristine. Resume normal |
| | doses in subsequent cycle once liver function has |
| | improved. No dose modifications to cisplatin required. |
| | Resume normal doses in subsequent cycle once liver |
| | function has improved. |
| Neurotoxicity | Omit vincristine if severe constipation or severe foot |
| | drop/ptosis. Resume at 66% dose in subsequent cycle |
| | if recovered. |
| Renal Function | If GFR ≤60 mL/min/m² then contact the Sponsor or the |
| | Medical Monitor. |
| Ototoxicity | Grade ≥3 omit cisplatin and contact Investigator. |
| Any Other Unresolved | To be discussed with the Sponsor or the CRO Medical |
| Grade ≥3 Toxicity | Monitor. |

6.2.2.5 Cycle A with Dinutuximab Beta

| Start | Day 41. | | | | | | | |
|---|---|---|---|---|---|---|---|---|
| Treatment Day | 1 | 2 | 3 | 4 | 5 | 6 | 7 | 8 |
| Vincristine, 1.5 mg/m²/d iv max 2 mg (bolus) | x | | | | | | | |
| Carboplatin, 750 mg/m²/d iv (1 h) | x | | | | | | | |
| Etoposide, 175 mg/m²/d iv (4 h) | x | x | | | | | | |
| Hydration 2000 mL/m²/d iv | x | | | | | | | |
| Hydration 1500 mL/m²/d iv/po | | x | x | x^{a} | | | | |
| G-CSF, 5 μg/kg/d sc | | | x | x | x | x | x | x |
| Dinutuximab beta (see also Table 11) | | | | | | | | |
| Infusion duration −1: | | x | x | | | | | |
| 10 mg/m²/d ctn (48 h) | | | | | | | | |
| Infusion duration 1: | | x | x | x | | | | |
| 10 mg/m²/d ctn (72 h) | | | | | | | | |
| Infusion duration 2: | | x | x | x | | | | |
| 10 mg/m²/d ctn (96 h) | | | | | | | | |

Abbreviations: ctn = continuous; G-CSF = granulocyte-colony stimulationg factor; iv = intravenous;
max = maximum; po = oral; sc = subcutaneous.
^{a}apply only when dinutuximab beta is given.

| Timing Within Cycle A | Vincristine 0 h 1.5 mg/m² (maximum dose 2 mg) as a | | |
|---|---|---|---|
| | single iv bolus. | | |
| | Carboplatin 0 h 750 mg/m² standard size bags infused | | |
| | over 1 hour in 5% dextrose. | | |
| | Etoposide 1 h 175 mg/m² standard size bags infused | | |
| | over 4 hours in 0.9% saline. | | |
| | Chemotherapy of Cycle A starts with vincristine bolus | | |
| | and immediate start of the carboplatin infusion (1 h) | | |
| | followed immediately by etoposide infusion (4 h). | | |
| | Start of gabapentin is variable and depends on the DL | | |
| | of dinutuximab beta. | | |
| | Gabapentin will start 3 days before the first dose of | | |
| | dinutuximab beta (Day −3). | | |
| | Dosing of gabapentin: 10 mg/kg/dose po. | | |
| | Day −3: 1 × 10 mg/kg po; Day −2: 2 × 10 mg/kg po; | | |
| | Day −1 3 × 10 mg/kg po. | | |
| | Gabapentin is tapered on first day after end | | |
| | dinutuximab beta infusion (Day +1). | | |
| | Day +1: 2 × 10 mg/kg po; Day +2 1 × 10 mg/kg po; | | |
| | Day +3 stop. | | |
| | Morphine continuous infusion (30 ug/kg/h) starts 1 | | |
| | hour before dinutuximab beta infusion. | | |
| | Morphine continuous infusion can be tapered starting | | |
| | from Day 2 of dinutuximab beta infusion. | | |
| | Antipyretic drug continuous co-medication and other | | |
| | supportive care measures for dinutuximab beta starts | | |
| | 1 hour before dinutuximab beta infusion. Refer to | | |
| | Appendix 3. | | |
| | Dinutuximab beta infusion starts at 2 mL/h (for | | |
| | details refer to Section 6.1). | | |
| Dose Adaptations for | Vincristine | 0.05 mg/kg/d iv | (bolus) |
| Children ≤12 kg | Carboplatin | 25 mg/kg/d iv | (1 h) |
| | Etoposide | 5.833 mg/kg/d iv | (4 h) |
| | Abbreviation: iv = intravenous. | | |
| Dose Modifications | | | |

| | |
|---|---|
| Infection | Grade 4: reduce carboplatin and etoposide doses to |
| | 75% of full-dose. Administer full-dose vincristine. |
| Hepatotoxicity | Do not alter doses for abnormal transaminases. If total |
| | bilirubin 26 to 50 μmol/L then give 50% dose |
| | etoposide, if total bilirubin ≥51 μmol/L omit etoposide |
| | and vincristine. Resume normal doses in subsequent |
| | cycle once liver function has improved. No dose |
| | modifications to carboplatin required. Resume normal |
| | doses in subsequent cycle once liver function has |
| | improved. |
| Neurotoxicity | Omit vincristine if severe constipation NCI-CTCAE |
| | version 5.0 Grade ≥3 or severe foot drop/ptosis. |
| | Resume at 66% dose in subsequent cycle if recovered. |
| Renal Function | No modifications required as long as normal urine |
| | output. |
| Any Other Unresolved | To be discussed with the Sponsor or the CRO Medical |
| Grade ≥3 Toxicity | Monitor. |

### 6.3 Preparation/Handling/Storage/Accountability

1. The investigator or designee must confirm appropriate temperature conditions have been maintained during transit for all dinutuximab beta received and any discrepancies are reported and resolved before use of the dinutuximab beta.
2. Only patients enrolled in the study may receive study treatment and only authorized study center staff may supply or administer study treatment. Dinutuximab beta must be stored in a secure, environmentally controlled, and monitored (manual or automated) area in accordance with the labeled storage conditions with access limited to the investigator and authorized study center staff.
3. The investigator, institution, or the head of the medical institution (where applicable) is responsible for dinutuximab beta accountability, reconciliation, and record maintenance (ie, receipt, reconciliation, and final disposition records).
4. Further guidance and information for the final disposition of unused dinutuximab beta are provided in the Pharmacy Manual.

The Investigator, a member of the study center staff, or a hospital pharmacist must maintain an adequate record of the receipt and distribution of all study medication using the Drug Accountability Form. These forms must be available for inspection at any time.

### 6.4 Measures to Minimize Bias: Randomization and Blinding

This is a nonrandomized open-label study.

### 6.5 Study Treatment Compliance

Study treatments will be administered to the patients under the supervision of appropriately trained staff at the study center.

### 6.6 Concomitant Therapy

The CRO Medical Monitor should be contacted if there are any questions regarding concomitant or prior therapy.

A list of excluded medications/therapy is provided in Appendix 2.

Neuropathic pain is an anticipated side effect of dinutuximab beta. Patients will receive gabapentin and IV morphine. If the patient does not experience significant pain during the first infusion of dinutuximab beta, the use of IV morphine can be modified per investigator discretion. Inflammatory side effects are anticipated that require co-medication with antipyretic drugs. Anti-histaminergic prophylaxis and prevention of gastritis is recommended. Instructions for the administration of these concomitant medications are included in Appendix 3.

### 6.6.1 Rescue Medicine

Dinutuximab beta must be administered by a healthcare professional prepared to manage severe allergic reactions including anaphylaxis. Infusion of dinutuximab beta must be initiated in an environment where full resuscitation services are immediately available.

### 6.7 Dose Modification

The dose modifications for chemotherapy toxicity can be found in Section 6.2.

### 6.7.1 General Guidance on Dose Modifications for Dinutuximab Beta

Previous experience with dinutuximab beta has shown a decrease of dinutuximab beta toxicity in subsequent cycles. Therefore, in this study, dinutuximab beta infusion flow rate may be reduced, or interrupted, in the first treatment cycle in accordance with the dose modifications that are described in this section. Patients who require more than a 2-day treatment interruption due to dinutuximab beta related toxicity may be discontinued from the study after discussion with the Sponsor or CRO Medical Monitor. If the infusion has been stopped or the flow rate decreased during the planned administration period, then the infusion may be returned up to the original infusion rate, but not higher, in accordance with the Section 6.7.1.1; treatment duration may be extended by 20% of the planned time period in order to complete the dose per cycle as planned (example: If 5 days=120 hours duration is planned, then prolongation up to 144 hours is allowed) (20% rule).

The total dose of dinutuximab beta that can be safely administered in each cycle will be monitored and considered by the DMC to define the recommended RP2D.

Dinutuximab beta dose modifications will be performed by changing the flow rate of the infusion or interrupting the infusion. This would usually occur in the event of Grade 3 or 4 toxicity as defined in NCI-CTCAE version 5.0.

### 6.7.1.1 Dinutuximab Beta Infusion Rate Modifications

Dinutuximab beta infusion rate modifications for dinutuximab beta related Grade 23 toxicities are as follows:
No modification for Grade ≥3 hematologic toxicity corrected by transfusion and no modification for Grade ≥3 leukocytopenia associated with chemotherapy.
For dinutuximab beta related Grade ≥3 toxicities, except as noted below, stop dinutuximab beta infusion.
   If toxicity resolves or improves to Grade ≤2 with supportive measures within 1 day, dinutuximab beta may be resumed at a 50% flow rate reduction (1 mL/h) to complete the prescribed dose. If the patient tolerates dinutuximab beta at a lower flow rate (ie, toxicity remains at Grade ≤2) for 12 hours, the dose may be increased to 100% flow rate (2 mL/h). If the infusion has been stopped or the flow rate decreased during the planned application period, the flow rate cannot exceed 2 mL/h, but treatment duration may be extended by 20% of the planned time period in order to complete the dose per cycle as planned (example: If 5 days=120 hours duration is planned, then prolongation up to 144 hours is allowed). If the full dose is not complete at the end of the 20% extension of time, the infusion should be stopped, and total dose infused at that point recorded.
   If toxicity (Grade ≥3) recurs while at 100% flow rate (2 mL/h) administration will be stopped and restarted at a 50% infusion rate (1 mL/h). If toxicity resolves or improves to Grade ≤2 with supportive measures, then the infusion of the prescribed dose at the lower rate will be completed. The flow rate cannot exceed 2 mL/h, but treatment duration may be extended by 20% of the planned time period in order to complete the dose per cycle as planned (example: If 5 days=120 hours duration is planned, then prolongation up to 144 hours is allowed).
   If toxicity (Grade ≥3) recurs while dinutuximab beta is administered at a lower flow rate, treatment will be discontinued for the cycle.
      6.7.2 Expected Toxicities that do not Require Dinutuximab Beta Infusion Rate Modification

The following expected toxicities will NOT need infusion rate modification when observed, provided that these toxicities are judged to be tolerable by the investigator, as well as the patient and parent or guardian.
Grade 3 pain.
Grade 3 fever.
Grade 3 nausea and vomiting and diarrhea.
Grade 2 skin toxicity (except stable Grade 3 urticaria that remains stable and tolerable, or improves with treatment [eg, iv diphenhydramine] within 24 hours).
Grade 3 electrolyte imbalance (especially hyponatremia <130 mmol/L in the absence of central nervous system [CNS] symptoms and sequelae) that improve with treatment within 24 hours.
Grade 3 hepatic toxicity that returns to Grade 1 prior to the time for next dinutuximab beta treatment course.
Grade 3 neurotoxicity with subjective findings only (eg, tingling, hot or cold hands, etc.)
Performance (30% to <50%, see Section 9.2.4).
Impaired visual accommodation correctable with spectacles with or without tinting.
Altered taste.

Specific product-related toxicities including guidance on early detection and management are described in Appendix 4.

### 6.7.3 Dinutuximab Beta Dose Escalation/De-Escalation

The dinutuximab beta dose escalation and de-escalation process (dose-finding process) is based on the evaluation of DLTs that occurred according to the BOIN method (19, 19).

Under the BOIN design, the decision to dose escalate, de-escalate, or remain at the same cumulative dose level is determined by comparing the observed DLT rate at the current cumulative dose level with a prespecified toxicity tolerance interval. If the observed DLT rate is less than or equal to the lower boundary of the interval, then the cumulative dose level is escalated. If the observed DLT rate is greater than or equal to the upper boundary of the interval, then the cumulative dose is de-escalated. If the observed DLT rate is within the interval, then the cumulative dose remains the same.

The target DLT rate for this clinical study is 33%, and the corresponding prespecified dinutuximab beta dose escalation and de-escalation boundaries are 0.260 (λe) and 0.395 (λd), respectively. The observed DLT rate used for determining whether the dose will escalate, de-escalate, or remain the same for the next cohort will be based on the number of patients experiencing DLTs during the time window defined above at the current dose schedule. If the observed DLT rate is:
≤0.260, the dinutuximab beta dose is escalated for the next cohort.
≥0.395, the dinutuximab beta dose is de-escalated for the next cohort.
0.269<x<0.395, the dinutuximab beta dose remains the same for the next cohort.

After the first 2 patients are enrolled and evaluated singly, patients will be enrolled and evaluated in cohorts of 3 patients. Note that a minimum of 2 evaluable patients tolerating a given cumulative dose level must be available before deciding to escalate to the next cumulative dose level. Evaluable patients have either completed the primary DLT observation period without a DLT or have experienced a DLT. All patients enrolled at a dose level should be evaluable before making a dosing change decision.

No dose skipping will occur with this design. If a patient is discontinued prior to completing the primary DLT observation period for any reason other than a DLT (and is therefore non-evaluable for the observed DLT rate calculations), then the patient will be replaced. The replacement patient will be assigned the same cumulative dose level as the non-evaluable patient.

Enrollment in the dose escalation phase will continue until the maximum sample size (25 patients in each of the GPOH and COJEC cohorts) is reached, unless required otherwise by the Study Stopping Rules defined below. The MTD will be defined as the cumulative dinutuximab beta dose level that in combination with chemotherapy has an estimated DLT rate closest to the target DLT rate.

### Dinutuximab Beta Study Stopping Rules

The dinutuximab beta dose-finding process for each chemotherapy regimen will be repeated until one of the following:
The predefined total number of evaluable patients is included.
The point estimate of toxicity probability for cumulative dose level-1 Is above 33%.
10 evaluable patients have been treated at the same cumulative dose level.

In case unacceptable toxicity or DLTs are observed early in the DLT assessment period with dinutuximab beta in cumulative dose level 1, cumulative dose level-1 dose modifications will be considered by the DMC.

### 6.8 Treatment After the End of the Study

After the end of the study (as defined in Section 4.4) the patients will continue under the care of their physician.

### 8.0 Discontinuation of Study Treatment and Subject Discontinuation/Withdrawal

### 8.1 Discontinuation of Study Treatment

As noted elsewhere, it is the responsibility of the DMC to monitor the nature and frequency of AEs that subjects experience on study.

Patients who discontinue study treatment in the dose escalation part of the study may be replaced (see Section 8.3); patients in the dose expansion part of the study will not be replaced.

See SoA table (Section 1.1) for data to be collected at the time of treatment discontinuation, follow-up, and for any further evaluations that need to be completed.

### 8.1.1 Dose-Limiting Toxicities

The evaluation of toxicity for the dose-finding process will be done during the first combination cycles of each induction chemotherapy regimen, so that each chemotherapy cycle is assessed once in combination with dinutuximab beta.

The DLT assessment period of the combination of chemotherapy with dinutuximab beta will be as follows:
GPOH with dinutuximab beta: the first 2 cycles of the combination (N6, N5); COJEC with dinutuximab beta: the first 3 cycles of the combination (course C, B, A).

See FIG. 1: Schematic representation of the study treatment cohorts of GPOH or COJEC induction chemotherapy regimens in combination with dinutuximab beta.

A DLT is defined as a dinutuximab beta related AE occurring during the DLT assessment period that leads to treatment discontinuation or meet 1 of the following criteria:
Grade ≥3 neurological toxicity.
Grade ≥3 cardiac toxicity.
Grade 4 severe, unrelenting neuropathic pain unresponsive to continuous infusion of narcotics and other adjuvant measures including lidocaine infusions.
Grade ≥3 capillary leak.
Grade 4 hyponatremia (<120 mmol/L) despite appropriate fluid management.
Grade 4 skin toxicity.
Grade 3 vision toxicity (ie, subtotal vision loss per toxicity scale).
Any Grade 4 toxicity (laboratory abnormality or non-hematological toxicity) that leads to change of management defined as: chemotherapy dose reduction or delay of next cycle (>7 days for COJEC regimen with dinutuximab beta or >14 days for GPOH regimen with dinutuximab beta).
Any Intensive care unit hospitalization for mechanical ventilation/ionotropic support/hemofiltration.
Any death not clearly related to disease progression (Grade 5 toxicity).

The NCI-CTCAE version 5.0 will be used for all severity grading. Prolongation of induction length and chemotherapy dose reduction (due to Grade ≤3 toxicity) will not be considered a DLT but will be monitored and considered by the DMC to define the RP2D.

### 8.2 Stopping Rules

### 8.2.1 Individual Patient Stopping Rules

Patients should stop dinutuximab beta and be taken off study treatment if the following toxicities occur:
Grade 3 (bronchospasm) and 4 (anaphylaxis) allergic reaction.
Grade ≥3 serum sickness.
Grade 4 severe, unrelenting neuropathic pain unresponsive to continuous infusion of narcotics and other adjuvant measures including Lidocaine infusions.
Any of the following neurotoxicities:
   Grade 3 sensory changes interfering with daily activities >2 weeks after completing dinutuximab beta therapy.
   Grade 3 objective motor weakness.
   Grade 3 vision toxicity (ie, subtotal vision loss per toxicity scale).
Grade 4 hyponatremia (<120 mmol/L) despite appropriate fluid management.
Grade 4 capillary leak syndrome (Grade 4 includes ventilator support).
Grade 4 skin toxicity.
Grade ≥3 cardiac toxicity.
Patients will be taken off protocol if International Neuroblastoma Response Criteria (INRC) defined disease progression occurs, see Appendix 6 (18).
Patients will be taken off protocol if the start of a treatment cycle including dinutuximab beta must be delayed for more than 1 week (COJEC regimen with dinutuximab beta) or more than 2 weeks (GPOH regimen with dinutuximab beta) due to toxicity. Unless there is agreement between the investigator and the Medical Monitor to continue the patient based on the reason for the delay, patient safety and wellbeing.

### 8.2.2 Study Stopping Rules

The following toxic events will trigger a hold in patient enrollment and review by the DMC If there are any patients with:
Grade 4 neurological toxicity.
Grade 4 cardiac toxicity.
Any treatment-related death (Grade 5 toxicity).

The DMC will be informed about any of the above toxic events. Patients on the study at that moment will continue study treatment.

The DMC will decide:
Continuation of the study with or without modification, or
Closure of treatment cohort, or
Termination of the entire study.
Modification in procedure related to patients currently on study.
   8.3 Patient Discontinuation/Withdrawal from the Study
The parent(s) or legal guardian may withdraw the patient from the study, at any time, or may be withdrawn at any time at the discretion of the investigator for safety, behavioral, compliance, or administrative reasons.
If the parent(s) or legal guardian withdraws consent, for disclosure of future information, the Sponsor may retain and continue to use any data collected before such a withdrawal of consent.
If the parent(s) or legal guardian withdraws a patient, from the study, he/she may request destruction of any samples taken and not tested, and the investigator must document this in the study center study records.
Progressive disease according to INRC (see Appendix 6).
Adverse events or toxicity requiring removal from protocol therapy.
Non-compliance that in the opinion of the investigator does not allow for ongoing participation.
Study is terminated by the Sponsor.
See SoA table (Section 1.1) for data to be collected at the time of study discontinuation, follow-up and for any further evaluations that need to be completed.

If a patient is considered as non-evaluable for the primary observation period, then enrollment of a new patient to the current cohort will be considered if there is less than the required number of evaluable patients.

If a patient is withdrawn from the study prior to completing 75% of the days of the DLT period without experiencing a DLT prior to withdrawal, then an additional patient may be added to that cumulative dose.

Patients receiving less than the 75% of the planned dose during the DLT period for treatment-related toxicity reasons will be reviewed by the Medical Monitor.

### 8.4 Lost to Follow-Up

The following actions must be taken if a patient fails to return to the clinic for a required study visit:
The study center must attempt to contact the patient and reschedule the missed visit as soon as possible and counsel the patient on the importance of maintaining the assigned visit schedule and ascertain whether the patient wishes to and/or should continue in the study.
Before a patient is deemed lost to follow-up, the investigator or designee must make every effort to regain contact with the patient (where possible, 3 telephone calls and, if necessary, a certified letter to the patient's last known mailing address or local equivalent methods). These contact attempts should be documented in the patient's medical record.
9.0 Should the patient continue to be unreachable, he/she will be considered to have withdrawn from the study.

### 8.0 Study Assessments and Procedures

Study procedures and their timing are summarized in Table 5 (GPOH) and Table 6 (COJEC).
Protocol waivers or exemptions are not allowed.
Immediate safety concerns should be discussed with the Sponsor immediately upon occurrence or awareness to determine if the patient should continue or discontinue study treatment.
Adherence to the study design requirements, including those specified in the SoA table (Section 1.1), is essential and required for study conduct.
All screening evaluations must be completed and reviewed to confirm that potential patients meet all eligibility criteria. The investigator will maintain a screening log to record details of all patients screened and to confirm eligibility or record reasons for screening failure, as applicable.
Procedures conducted as part of the patient's routine clinical management (eg, blood count) and obtained before signing of the ICF may be utilized for screening or baseline purposes provided the procedures met the protocol specified criteria and were performed within the time frame defined in the SoA table (Section 1.1).
The maximum amount of blood collected from each patient over the duration of the study will be included in the ICF and Laboratory Manual.

### 9.1.1 Screening

The screening period starts with the date of signature of the informed consent and needs to be completed within 21 days.

Tests that are required to establish the diagnosis of Stage M neuroblastoma and other examinations done before signature of the consent form that are required in the screening period will not have to be repeated. Patients referred to a study center with acceptable imaging quality (to be assessed by the study center) do not have to repeat the examinations.

### 9.1.1.1 Assessment of Basic Patient Characteristics

Complete medical/surgical history.
Concomitant medication.
Physical examination.
Vital signs (blood pressure, pulse rate, body temperature).
Body weight.
Body height.
Performance status.
Pain assessment (see Appendix 5).

### 9.1.1.2 Disease Assessments

Tests that were not performed to assess the disease in the pre-screening period need to be completed in order to have the full set of disease assessment investigations available.
Imaging of the primary tumor (computed tomography [CT]/magnetic resonance imaging [MRI]).
Imaging of the brain (CT/MRI).
Whole-body ¹²³I-metaiodobenzylguanidine single-photon emission tomography (¹²³I-MIBG SPECT).
Two bone marrow aspirates.
Two bone marrow trephine biopsies.
Urinary catecholamine metabolites.
Tumor biopsy and histology.
   If diagnosis of neuroblastoma is established by presence of neuroblastoma cells in the bone marrow along with elevated urine catecholamines rather than biopsy of the primary tumor, then tumor biopsy and histology is not required.
Determination of MYCN amplification.
Serum lactate dehydrogenase (LDH) and ferritin levels.

Some tests for these disease assessments may not be complete before it is necessary for the patient to start with the first chemotherapy cycle (without dinutuximab beta). In particular, histopathology, immunohistochemistry, MYCN status and occasionally MIBG scans may take more time and may be performed during start of induction chemotherapy (without dinutuximab beta).

In case of MIBG-negative neuroblastoma, [¹⁸F]-fluorodeoxyglucose-positron emission tomography should be performed.

These assessments follow the revised international Neuroblastoma Response Criteria (18).

### 9.1.1.3 Baseline Organ Function and Laboratory Tests

Cardiac function will be assessed by echocardiography and 12-lead ECG to determine the ejection fraction and the cardiac rhythm. Baseline is important to assess expected cardiac toxicity from anthracyclines and unexpected toxicity.
Hearing test is important at baseline to assess ototoxicity in particular with cisplatin.
Pulmonary function test (PFT) will be done on patients able to comply with the test. Oxygen saturation at room air can replace PFT in patients who cannot comply with the PFT. This important baseline information is also required for long-term toxicity assessment after BuMel high-dose chemotherapy (HDC).
Full blood count (hemoglobin, platelets, white blood cells with differential [neutrophils, lymphocytes, eosinophils, basophils]) and biochemistry including creatinine and/or cystatin C clearance, sodium, potassium, chloride, ALT, AST, total bilirubin, creatinine and/or cystatin C, C-reactive protein (CRP) will evaluate hematologic, hepatic and renal baseline characteristics.
Pregnancy test in female patients of childbearing potential.
Human Immunodeficiency virus (HIV), Hepatitis B and Hepatitis C infection.

Tests that were performed in the pre-screening period do not need to be repeated in the screening period. These tests should be completed before starting the first chemotherapy cycle.

9.1.1.4 Baseline Status of immune Parameters

In order to compare immunomodulation by the study treatment to baseline, the assessment of the following parameters is required at baseline:
Immunophenotype.
Genotyping, polymorphisms, killer immunoglobulin-like receptor (KIR)/KIR-Ligand mismatch.
HACA, Complement-dependent Cytotoxicity (CDC) and ADCC.

### 9.1.2 Assessment Prior to Start of Chemotherapy Cycles Without Dinutuximab Beta

Physical examination.
Vital signs (blood pressure, pulse rate, body temperature).
Body weight.
Performance status.
Pain assessment.
Full blood count: hemoglobin, platelets, white blood cells with differential (neutrophils, lymphocytes, eosinophils, basophils).
Biochemistry: sodium, potassium, chloride, ALT, AST, total bilirubin, creatinine and/or, cystatin C, CRP.
Pregnancy test in female patients of childbearing potential.
AEs.
Concomitant medication.
Cardiac function will be assessed by echocardiography and 12-lead ECG to determine the ejection fraction and the cardiac rhythm. Baseline is important to assess expected cardiac toxicity from anthracyclines and unexpected toxicity.
Hearing test.
Creatinine and/or cystatin C clearance.

These assessments should be available prior to first dosing of chemotherapy on Day 1. Information can be collected up to 96 hours prior to first dosing.

9.1.3 Assessment Prior to Start of Chemotherapy Cycles with Dinutuximab Beta

The following safety assessments will be available prior to first dosing of chemotherapy on Day 1. Information can be collected up to 96 hours prior to first dosing of chemotherapy. Information is required to assess, if the treatment can be given, if dose adaptation is required and to calculate the drug doses.
Physical examination.
Vital signs (blood pressure, pulse rate, body temperature).
Performance status.
Full blood count: hemoglobin, platelets, white blood cells with differential (neutrophils, lymphocytes, eosinophils, basophils).
Biochemistry: sodium, potassium, chloride, ALT, AST, total bilirubin, creatinine and/or cystatin C, and CRP.
Pregnancy test in female patients of childbearing potential.
Echocardiography and 12-lead ECG prior to anthracycline-containing chemotherapy cycles.
Hearing test prior to cisplatin- or carboplatin-containing chemotherapy cycles.

The following procedures will be done on Day 1 of the cycle prior to the first dose of chemotherapy:
Physical examination.
Vital signs (blood pressure, pulse rate, body temperature).
Performance status.
Pain assessment (see Appendix 5).
AEs.
Concomitant medication must be documented.

### 9.1.3.1 Assessment During Chemotherapy Without Dinutuximab Beta

Daily physical examination.
Daily vital signs (blood pressure, pulse rate, body temperature).
Daily body weight.
Daily documentation of performance status.
Daily pain assessment (see Appendix 5).
Daily AE assessment.
Daily documentation of concomitant medication.

Daily assessments start on Day 1 and continue until last day of chemotherapy.
Full blood count and biochemistry on last day of chemotherapy. Full blood count: hemoglobin, platelets, white blood cells with differential (neutrophils, lymphocytes, eosinophils, basophils). Biochemistry: sodium, potassium, chloride, ALT, AST, total bilirubin, creatinine and/or cystatin-C, and CRP.

### 9.1.3.2 Assessment During Chemotherapy With Dinutuximab Beta

Daily physical examination.
Daily vital signs (blood pressure, pulse rate, body temperature).
Daily body weight.
Daily documentation of performance status.
Daily pain assessment (see Appendix 5).
Daily AE assessment.
Daily documentation of concomitant medication.

Daily assessments start on Day 1 and continue until last day of chemotherapy or last day of dinutuximab beta, whichever comes last.
Start and end of dinutuximab beta infusion: Full blood count and biochemistry: Full blood count: hemoglobin, platelets, white blood cells with differential (neutrophils, lymphocytes, eosinophils, basophils). Biochemistry: sodium, potassium, chloride, ALT, AST, total bilirubin, creatinine and/or cystatin C, CRP.
Immunophenotyping: take 2 samples: on first day (prior dosing) and last day of dinutuximab beta infusion (end-of-infusion).
Samples for PK, CDC and ADCC must be taken on first day (prior dosing) and last day of dinutuximab beta Infusion (end-of-infusion).
HACA.
   9.1.3.3 Assessment after Chemotherapy without and with Dinutuximab Beta

Adverse event and concomitant medication documentation will be done once after completion of chemotherapy or dinutuximab beta on the visit prior to the next cycle.

9.1.3.4 Mid-Induction Treatment Evaluation Disease evaluations during treatment include the following tests:
¹²³I-MIBG SPECT.
Bilateral bone marrow aspirate (repeat also when negative at baseline).
Bilateral bone marrow trephine biopsy (repeat also when negative at baseline).
Urinary catecholamine metabolites.

Replace ¹²³I-MIBG SPECT by [¹⁸F]-fluorodeoxyglucose-positron emission tomography in patients with MIBG-negative neuroblastoma.

### Time Points:

GPOH induction with dinutuximab beta: After the first N6 chemotherapy cycle. Before the second N5 cycle.

COJEC induction with dinutuximab beta: After the second B-chemotherapy cycle. Before the second A chemotherapy cycle.
Physical examination.
Vital signs (blood pressure, pulse rate, body temperature).
Body weight.
Documentation of performance status.
AE assessment.
Documentation of concomitant medication.
Full blood count and biochemistry.
Full blood count: hemoglobin, platelets, white blood cells with differential (neutrophils, lymphocytes, eosinophils, basophils).
Biochemistry: sodium, potassium, chloride, ALT, AST, total bilirubin, creatinine and/or cystatin C, CRP.
Echocardiography.
   9.1.3.5 End of induction Treatment Evaluation (=End of induction)

Disease evaluations end of induction prior to HDC include the following tests, time period of 4 weeks prior to start of conditioning regimen:
¹²³I-MIBG SPECT.
MRI/CT (1. brain, also when negative at baseline; 2. primary tumor).
Bilateral bone marrow aspirate (repeat also when negative at baseline).
Bilateral bone marrow trephine biopsy (repeat also when negative at baseline).
Urinary catecholamine metabolites.

Replace ¹²³I-MIBG SPECT by [¹⁸F]-fluorodeoxyglucose-positron emission tomography in patients with MIBG-negative neuroblastoma at initial investigation.

Further safety assessments are required:
Physical examination.
Vital signs (blood pressure, pulse rate, body temperature).
Body weight.
Documentation of performance status.
AEs.
Documentation of concomitant medication.
Echocardiography.
12-lead ECG.
Hearing test.
PFT/O₂ saturation on room air.
Full blood count and biochemistry:
Full blood count: hemoglobin, platelets, white blood cells with differential (neutrophils, lymphocytes, eosinophils, basophils).
Biochemistry: sodium, potassium, chloride, ALT, AST, total bilirubin, creatinine and/or cystatin C, CRP.
Pregnancy test in female patients of childbearing potential.
HACA.

For patients who do not continue on to HDC after induction and proceed with alternative therapy due to refractory disease, the end of induction visit will also serve as the end-of-study visit. Post-study cancer treatment, including but not limited to maintenance treatment, details (normally type and amount/duration of cancer treatment) will be recorded.

### 9.1.3.6 Evaluation 100 Days After HDC (End-of-Study)

On Day 100 after the HDC visit is the end-of-study. The time point of assessments may fall in a time window of ±2 weeks around the exact Day 100 after HDC.

Disease evaluations 100 days after HDC include the following tests:
¹²³I-MIBG SPECT.
MRI/CT (1. brain, also when negative at baseline; 2. primary tumor).
Bilateral bone marrow aspirate (repeat also when negative at baseline).
Bilateral bone marrow trephine biopsy (repeat also when negative at baseline).
Urinary catecholamine metabolites.

Replace ¹²³I-MIBG SPECT by [¹⁸F]-fluorodeoxyglucose-positron emission tomography in patients with MIBG-negative neuroblastoma at initial investigation.

Further safety assessments are required:
Physical examination.
Vital signs (blood pressure, pulse rate, body temperature).
Body weight.
Documentation of performance status.
AEs.
Documentation of concomitant medication.
Echocardiography.
12-lead ECG.
Hearing test.
PFT/O₂ saturation on room air.
Full blood count and biochemistry:
Full blood count: hemoglobin, platelets, white blood cells with differential (neutrophils, lymphocytes, eosinophils, basophils).
Biochemistry: sodium, potassium, chloride, ALT (SGPT), AST (SGOT), total bilirubin, creatinine and/or cystatin C, and CRP.
Pregnancy test in female patients of childbearing potential.
HACA: Take 1 sample. Process sample and store.

### 9.2 Efficacy Assessments

Planned time points for all efficacy assessments are provided in the SoA table (Section 1.1).

Imaging, bone marrow aspirates/biopsies and blood assessments will be performed to assess tumor response. The investigator is responsible for the determination of the patient's response per the, see Appendix 6. Tumor response parameters that will be assessed include the following:
Best overall response (CR, partial response [PR], minor response [MR], stable disease [SD] [lasting 12 weeks], or disease progression).
ORR (CR+PR).
Clinical benefit rate (CBR) (CR or PR or MR or SD [lasting at least 12 weeks]).
Duration of response or duration of SD.

Time-to-event endpoints of time to progression, progression-free survival, and OS during study observation period will also be assessed.

### 9.2.1 Imaging

At screening, patients will undergo a whole-body ¹²³I-MIBG SPECT scan. For patients with MIBG-negative neuroblastoma at the initial investigation the ¹²³I-MIBG SPECT scan will be replaced with [¹⁸F]-fluorodeoxyglucose-positron emission tomography in patients at subsequent assessments.

These assessments follow according to the revised INRC (see Appendix 6).

Brain and primary tumor MRI or CT will be performed as indicated. Contrast enhanced brain MRI is preferred; however, if MRI contrast is contraindicated, then MRI without contrast or CT with/without contrast is acceptable.

### 9.2.2 Bone Marrow Aspirate and Biopsy

For detection and quantification of tumor cells in bone marrow and fine-needle aspirates, anti-GD2 antibodies used for bone marrow diagnostics, as well as NB84a, anti-CD56 and anti-S-100 are recommended for material obtained by fine-needle aspiration.

### 9.2.3 Pain Grading

Face pain scales will be used for the patient on questioning from a parent or guardian to indicate the level of pain the patient feels at the time points during the study. Pictures to guide in the grading on a scale of 0 to 10 in increments of 2, where 0 is no pain and 10 is very much pain, will be included in the patient diary.

In addition, pain assessments scores will be evaluated by the parent or guardian and/or the investigator by use of the pain assessment scores included in Appendix 5.

### 9.2.4 Performance Status

Performance of daily activities will be assessed by use of the Lansky Play-Performance scale for patients <16 years-old and the Karnofsky Performance Scale for patient >16 years-old.

### 9.3 Safety Assessments

Planned time points for all safety assessments are provided in Table 5 (GPOH) and Table 6 (COJEC).

### 9.3.1 Demographics and Medical History

Demographics and relevant medical and surgical history will be recorded in the CRF.

### 9.3.2 Physical Examinations

At screening a complete physical examination will include at a minimum, assessments of the cardiovascular, respiratory, gastrointestinal, and neurological systems.
At subsequent assessments brief physical examination will include at a minimum, assessments of the skin, lungs, cardiovascular system, and abdomen (liver and spleen).
A hearing test will be conducted to capture ototoxicity.
Investigators should pay special attention to clinical signs related to previous serious illnesses.

### 9.3.3 Vital Signs

Oral temperature, pulse rate, and blood pressure will be assessed.
Blood pressure and pulse measurements will be assessed with a completely automated device. Manual techniques will be used only if an automated device is not available.
Blood pressure and pulse measurements should be preceded by at least 5 minutes of rest for the patient in a quiet setting without distractions (eg, television, cell phones).
Vital signs (to be taken before blood collection for laboratory tests) will consist of 1 pulse and 3 blood pressure measurements.

### 9.3.4 Height and Weight

Height (screening only) and body weight will be measured and recorded according to the study center's standard procedures.

### 9.3.5 Echocardiogram

Cardiac function will be evaluated by echocardiogram at screening/baseline. Additional cardiac imaging may be performed if indicated by clinical signs or symptoms. The same imaging modality should be used for the additional imaging.

### 9.3.6 Electrocardiograms

Single 12-lead ECG will be obtained as outlined in the SoA table (Section 1.1) using an ECG machine that automatically calculates the heart rate and measures PR, QRS, QT, and QTcF (QT corrected for Fridericia's formula) intervals.

### 9.3.7 Pulmonary Function Test

Pulmonary function test will be performed on patients able to comply with the test. Oxygen saturation at room air can replace PFT in patients who cannot comply with the PFT. This important baseline information is required also for long-term toxicity assessment after HDC BuMel administration.

### 9.3.8 Clinical Safety Laboratory Assessments

Clinical laboratory tests are to be performed—see the SoA table (Section 1.1) for the timing and frequency.
The Investigator must review the laboratory report, document this review, and record any clinically significant (CS) changes occurring during the study in the AE section of the CRF. The laboratory reports must be filed with the source documents. Clinically significant abnormal laboratory findings are those that are not associated with the underlying disease, unless judged by the investigator to be more severe than expected for the patient's condition.
All laboratory tests with values considered clinically significantly abnormal during participation in the study or within 30 days after the last dose of dinutuximab beta should be repeated until the values return to normal or baseline or are no longer considered CS by the Investigator or Medical Monitor.
   If such values do not return to normal/baseline within a period of time judged reasonable by the investigator, the etiology should be identified, and the Sponsor notified.
   All protocol-required laboratory assessments must be conducted in accordance with the Laboratory Manual and the SoA (Section 1.1).
   If laboratory values from non-protocol specified laboratory assessments performed at the Institution's local laboratory require a change in patient management or are considered CS by the investigator (eg, SAE or AE or dose modification), then the results must be recorded in the CRF.

Abnormal laboratory values or test results constitute AEs only if they induce clinical signs or symptoms, are considered CS, require therapy (eg, hematologic abnormality that requires transfusion), or require discontinuation of dinutuximab beta or chemotherapy. Wherever possible, the reporting investigator will use the clinical, rather than the laboratory, term (eg, anemia versus low hemoglobin value).

### 9.4 Adverse Events

Adverse events will be reported by the patient (or, when appropriate, by a caregiver, surrogate, or the participant's legally authorized representative).

The Investigator and any designees are responsible for detecting, documenting, and recording events that meet the definition of an AE or SAE and remain responsible for following-up AEs that are serious, considered related to the study treatment or study procedures, or that caused the patient to discontinue the study treatment (see Section 8.0).

9.4.1 Time Period and Frequency for Collecting AE and SAE information

All AEs and SAEs will be collected from the signing of the ICF until end-of-treatment at the time points specified in the SoA table (Section 1.1).

Signs and symptoms that begin before the start of study treatment but after obtaining informed consent will be recorded on the medical history/current medical conditions section of the CRF, not the AE section.

All SAEs will be recorded and reported to the Sponsor or designee within 24 hours. The investigator will submit any updated SAE data to the Sponsor within 24 hours of it being available.

Investigators are not obligated to actively seek AE or SAE after conclusion of the study participation. However, if the investigator learns of any SAE, including a death, at any time after a patient has been discharged from the study, and he/she considers the event to be reasonably related to the study treatment or study participation, the investigator must promptly notify the Sponsor.

### 9.4.2 Method of Detecting AEs and SAEs

Care will be taken not to introduce bias when detecting AEs and/or SAEs. Open-ended and non-leading verbal questioning of the patient is the preferred method to inquire about AE occurrences.

### 9.4.3 Follow-Up of AEs and SAEs

After the initial AE/SAE report, the investigator is required to proactively follow each patient at subsequent visits/contacts. All SAEs, will be followed until resolution, stabilization, the event is otherwise explained, or the patient is lost to follow-up (as defined in Section 8.4).

### 9.4.4 Regulatory Reporting Requirements for SAEs

Prompt notification by the investigator to the Sponsor of an SAE is essential so that legal obligations and ethical responsibilities toward the safety of patients and the safety of a study treatment under clinical investigation are met.
The Sponsor has a legal responsibility to notify both the local regulatory authority and other regulatory agencies about the safety of a study treatment under clinical investigation. The Sponsor will comply with country-specific regulatory requirements relating to safety reporting to the regulatory authority, institutional Review Boards (IRB)/Independent Ethics Committees (IEC), and investigators.
Investigator safety reports must be prepared for suspected unexpected serious adverse reactions (SUSAR) according to local regulatory requirements and Sponsor policy and forwarded to investigators as necessary.
An Investigator who receives an investigator safety report describing an SAE or other specific safety information (eg, summary or listing of SAEs) from the Sponsor will review and then file it along with the Investigator's Brochure and will notify the IRB/IEC, if appropriate according to local requirements.

### 9.4.5 Pregnancy

Details of all pregnancies in [female patients and, if Indicated, female partners of male patients] will be collected after the start of dinutuximab beta and until 1 year after stopping dinutuximab beta.
If a pregnancy is reported, the investigator should inform the Sponsor within 24 hours of learning of the pregnancy.
Abnormal pregnancy outcomes (eg, spontaneous abortion, fetal death, stillbirth, congenital anomalies, and ectopic pregnancy) are considered SAEs.

### 9.5 Treatment of Overdose

No cases of dinutuximab beta overdose have been reported. In case of overdose, patients should be carefully observed for signs or symptoms of adverse reactions and supportive care administered, as appropriate.

In addition, the investigator should:
Contact the CRO Medical Monitor Immediately.
Closely monitor the patient for any AE/SAE and laboratory abnormalities until dinutuximab beta can no longer be detected systemically.
Obtain a plasma sample for PK analysis if requested by the CRO Medical Monitor (determined on a case-by-case basis).
Document the quantity of the excess dose as well as the duration of the overdose in the CRF.
Record the batch number of dinutuximab beta in AE reports and reports of overdose.

Decisions regarding dosing interruptions or dose modifications will be made by the investigator in consultation with the CRO Medical Monitor based on the clinical evaluation of the patient.

Any overdose of COJEC and GPOH induction treatment will be recorded.

### 9.6 Pharmacokinetics

### 9.6.1 Collection of Blood Samples for Dinutuximab Beta Concentration Determination

Blood samples will be taken either by direct venipuncture or an indwelling cannula inserted in a forearm vein, or a double-lumen central line if inserted. Blood samples collected predose and on the same day of dose administration should may be collected in the contralateral (opposite) arm from the one being used for drug infusion.

If a single-lumen catheter is used, draw 5 mL blood before taking the samples. Flush with 10 mL saline if allowed by institutional standards.

Sampling must be done with caution in accordance with institutional standards when working on central venous lines.

Acceptable collection time windows from nominal collection times are included in Table 12. Additional detailed instructions for the blood collection (including but not limited to blood volume per sample), processing, storage, and shipment to the bioanalytical laboratory will be described in the Laboratory Manual.

The actual date and time (24-hour clock time) of each sample collection will be recorded along with the start date/time and end date/time of the infusion of study treatment (as well as start and end time of any interruptions, if present).

TABLE 12
Blood Sample Collection for Dinutuximab
Beta Concentration Determination

| Course of Therapy | Sample | Nominal PK Sample | Collection Time |
|---|---|---|---|
| (Infusion Duration) | Name | Collection Time | Window |
| 2 days (of 24-hour | Predose | Predose (0 h) | −2 hours |
| infusions) | End-of- | End of 2^{nd} infusion | ±30 minutes |
| | Infusion | (48 h) | (1.5 to 2.5 hours |
| | | +2 hours | post EOI) |
| 3 days (of 24-hour | Predose | Predose (0 h) | −2 hours |
| infusions) | End-of- | End of 3^{rd} infusion | ±30 minutes |
| | Infusion | (72 h) | (1.5 to 2.5 hours |
| | | +2 hours | post EOI) |
| 4 days (of 24-hour | Predose | Predose (0 h) | −2 hours |
| infusions) | End-of- | End of 4^{th} infusion | ±30 minutes |
| | Infusion | (96 h) | (1.5 to 2.5 hours |
| | | +2 hours | post EOI) |
| 5 days (of 24-hour | Predose | Predose (0 h) | −2 hours |
| infusions) | End-of- | End of 5^{th} infusion | ±30 minutes |
| | Infusion | (120 h) | (1.5 to 2.5 hours |
| | | +2 hours | post EOI) |
| 7 days (of 24-hour | Predose | Predose (0 h) | −2 hours |
| infusions) | End-of- | End of 7^{th} infusion | ±30 minutes |
| | Infusion | (168 h) | (1.5 to 2.5 hours |
| | | +2 hours | post EOI) |

Abbreviation: EOI = end-of-infusion; PK = pharmacokinetics.

### 9.6.2 Determination of Dinutuximab Beta Concentrations in Serum PK Samples

Samples for the determination of dinutuximab beta concentrations in serum will be analyzed by a bioanalytical laboratory under the responsibility of the Sponsor using appropriate validated bioanalytical methods.

Samples that meet the criteria of the bioanalytical laboratory's Standard Operating Procedures (SOPs) (ie, condition upon receipt, stability, etc.) will be analyzed in accordance with the bioanalytical laboratory's SOP(s), the validated method, and the bioanalytical sample analysis plan. Full details of the bioanalytical methods and batch performance will be described in a separate Bioanalytical Report.

### 9.6.3 Calculation of Pharmacokinetic Parameters

Only sparse samples (predose and end-of-Infusion) will be collected. As such, noncompartmental analysis will not be conducted as part of this study.

### 9.7 Pharmacodynamics

### 9.7.1 Immunophenotyping

A blood sample (2 to 3 mL) will be collected in a sample tube containing ethylenediaminetetraacetic acid (EDTA). The actual date and time (24-hour clock time) of each sample collection will be recorded. The details of blood sample processing, storage and shipment will be described in the Laboratory Manual.

Flow cytometry analyses will be performed according to local procedures. It is recommended to use the whole blood lysis technique in order to prevent cell loss during sample preparation.

The Immunophenotypic analysis will be done at laboratories of participating study centers and will include 3 populations:
NK-cells (CD16+/CD56+NK-cells).
CD8 T-cells (CD8+/CD3+ T-cells).
CD4 T-cells (CD4+/CD3+ T-cells).

Data will be analyzed locally, and results recorded as absolute cell numbers per μL of the subpopulations.

### 9.8 Genetics

### 9.8.1 Fc-Gamma Receptor Polymorphisms and KIR/KIR-Ligand Analysis

A blood sample (2 mL) will be collected in a sample tube containing EDTA on Cycle 1 Day 1 (minimum 2 mL) at the times included in Table 5 and Table 6. The actual date and time (24-hour clock time) of each sample collection will be recorded. The blood sample should be sent for analysis within 24 hours. The details of blood sample processing, storage, and shipment will be described in the Laboratory Manual.

### 9.8.2 Biomarkers.

Blood samples for the measurements of biomarkers including HACA, CDC and ADCC will be collected at the times included in Table 5 and Table 6. Details of biomarker sample collection, preparation, handling, storage, and shipment will be included in a separate Laboratory Manual.

### 9.0 Statistical Considerations

### 9.1 Statistical Hypotheses

The objectives of this study are to determine the recommended infusion duration and cumulative dose levels of dinutuximab beta when combined with different induction chemotherapy regimens (GPOH or COJEC) for the treatment of newly diagnosed high-risk neuroblastoma patients and to estimate rates of OS, EFS, toxicity as well as other endpoints for the treated population. Given the lack of an internal control group, formal hypothesis testing will not be performed. Ad hoc comparisons of event rates to historical controls may be specified in later statistical analysis plans (SAPs).

### 9.2 Sample Size Determination

This is a Phase 1 study with an open-label dose escalation phase followed by a single-cohort expansion at the (chosen) cumulative dose level in 2 separate cohorts. A BOIN adaptive procedure will be used to assign patients to cumulative dose levels and determine the MTD. To obtain the escalation and de-escalation boundaries for this study, the function “get.boundary” within the R Package “BOIN” was used. The specific function call was as follows:

get.boundary(0.33, 9, 3, n.earlystop=100, p.saf=0.6*0.33, p.tox=1.4*0.33, cutoff.eli=0.95, extrasafe=FALSE, offset=0.05)

Where the arguments are defined as follows:

| | |
|---|---|
| target | 0.33 = the target DLT rate |
| ncohort | 9 = the total number of cohorts (to arrive at N > 25) |
| cohortsize | 3 = the cohort size |
| n.earlystop | 100 = n.earlystop~100 essentially turns |
| | off this type of early stopping. |
| p.saf | 0.6*target rate = the highest toxicity |
| | probability that is deemed |
| | subtherapeutic (ie, below the MTD) such that |
| | dose escalation should be |
| | made. The default value is p.saf = 0.6 * target. |
| p.tox | 1.4*target rate = the lowest toxicity |
| | probability that is deemed overly toxic |
| | such that de-escalation is required. |
| | (The default value is p.tox = 1.4*target.) |
| cutoff.eli | 0.95 = the cutoff to eliminate an overly |
| | toxic dose for safety. (The default |
| | value = 0.95 for general use.) |
| extrasafe | FALSE. set extrasafe = TRUE to impose a |
| | stricter stopping rule for extra |
| | safety, expressed as the stopping boundary |
| | value in the result. |
| offset | 0.05 = not used when extrasafe = FALSE |

A maximum sample size of 25 evaluable patients each is planned for the dose escalation phase in each cohort. In the expansion phase, 10 additional patients will be enrolled at the [chosen] cumulative dose level in each cohort. The maximum number of patients in this clinical study will be 70.

The sample size is not based on statistical considerations but is typical for studies of this nature and is considered adequate to characterize the distribution of the planned endpoints. Any statistical testing will be considered exploratory and descriptive.

### 9.3 Populations c hr Analyses

For purposes of analysis, the analysis sets are defined in Table 13.

TABLE 13
Analysis Sets

| Analysis Set | Description |
|---|---|
| Screened Analysis Set | All patients who sign the informed consent form (ICF). |
| Enrolled Study | All patients in the Screened Analysis Set who are assigned to |
| Treatment Analysis Set | study treatment. |
| Full Efficacy Analysis Set | All enrolled patients who receive at least 1 dose of |
| | dinutuximab beta and have specific postbaseline efficacy |
| | assessments available. |
| Safety Analysis Set | All enrolled patients who receive at least 1 dose of study |
| | treatment (ie, chemotherapy) will be included in the analysis |
| | of safety parameters and immunogenicity. Patients will be |
| | analyzed according to the treatment they received. |
| Pharmacokinetic (PK) | Patients in the safety analysis set who have at least 1 |
| Analysis Set | quantifiable PK concentration available at a scheduled time |
| | postdose, which is not expected to be significantly affected |
| | by an important protocol deviation/violation or event. |

### 9.4 Statistical Analyses

The SAP will be developed and finalized before database lock and will describe the patient analysis sets to be included in the analyses, and procedures for accounting for missing, unused, and spurious data. This section is a summary of the planned statistical analyses of the primary and secondary endpoints.

The analysis and reporting of the exploratory and ancillary endpoints will be described in the SAP.

### 9.4.1 Safety Analyses

All safety analyses will be performed on the safety analysis set.

Safety, tolerability, and determining the MTD of dinutuximab beta in each chemotherapy combination is the primary objective of the study and will be assessed primarily through the incidence of DLTs during (and after) the primary DLT observation period of the escalation phase. The recommended RP2D will be determined subsequently based on MTD during the dose escalation phase but may be modified with additional safety experience observed during the expansion phase.

A BOIN design will be utilized to help identify the MTD. See Section 6.7.3 for details regarding calculation of the observed DLT rate and the criteria for escalating, de-escalating, or keeping the cumulative dose level dinutuximab beta the same. The MTD will be the cumulative dose level in combination with chemotherapy which according to DMC assessment is best tolerated and has a DLT rate closest to the target rate of 33%.

The RP2D will be determined by the MTD or the maximum administered cumulative dose level if no MTD dose is reached.

Each patient's outcome with respect to DLTs will be used to update the BOIN algorithm and these results will be used by the DMC to allocate additional patients to a cumulative dose level (see DMC charter).

At the conclusion of the dose escalation and expansion phases, safety outcomes will be reported by cumulative dose level, cohort, and regimen within cohort up to the end-of-treatment.

Frequency of DLTs (escalation phase only) as used for dose escalation and de-escalation decisions will be tabulated.

Safety and tolerability will be based primarily on AEs. The verbatim description of each reported AE will be coded to a standardized description (ie, preferred term [PT]) and system organ class [SOC] using the current version of the Medical Dictionary for Regulatory Activities [MedDRA]).

Adverse events will be tabulated by SOC and PT and will be further categorized by maximum severity. The total number of each AE and the number and percentage of patients experiencing each AE will be presented by study phase (dose escalation and expansion). For the dose escalation phase, the data will be further categorized by cumulative dose level and overall (total across all cumulative dose levels). In addition, the AEs may be summarized by cumulative dose level (combined dose escalation and expansion phases).

Tabular summaries will be prepared for all AEs (as a whole), as well as for the following subcategories of AEs: SAEs, AE's related to dinutuximab beta, AEs leading to study treatment discontinuation, and AEs leading to death.

All reported AEs will be listed by patient, including verbatim description, PT, SOC, onset date, end date, severity, whether considered an SAE, relationship to study dinutuximab beta, expectedness, action taken related to study drugs, and outcome. Focused listings for SAEs, AEs leading to study treatment discontinuation, AEs leading to death, and ADRs (adverse drug reactions) will also be generated.

Secondary safety and tolerability assessments will be summarized descriptively by study phase (dose escalation and expansion). For the dose escalation phase, the data will be further categorized by cumulative dose level.

Tolerability assessments will include reporting the following-up to the last safety follow-up visit:
Frequency of study discontinuation and reason.
Frequency of dose interruption and reduction.
Mean and median total dose of dinutuximab beta administered over the induction period.

All laboratory test results, pain assessments, vital signs measurements, ECG results, weight, and body mass index will be summarized for each treatment group using descriptive statistics at each visit for raw numbers and change from baseline. Screening will serve as baseline for calculating changes from baseline.

Physical examination findings will be summarized by body system using a shift table from baseline to each subsequent assessment time point and categories of normal, abnormal not clinically significant (NCS), and abnormal CS.

The performance status and body weight tabular summaries will include mean, standard deviation, median, and range for each assessment time point, as well as for the calculated changes from baseline to each subsequent time point.

Daily pain assessment scores will be summarized in both tabular and graphic format. The tabular summaries will include mean, standard deviation, median, and range for each assessment time point, as well as for the calculated changes from baseline to each subsequent time point. The figures will include mean and standard deviation over time.

Vital signs will be summarized in both tabular and graphic format. The tabular summaries will include mean, standard deviation, median, and range for each assessment time point, as well as for the calculated changes from baseline to each subsequent time point. The figures will include mean and standard deviation for each vital sign over time.

The tabular summary of 12-lead ECG data, ie, ventricular rate, RR interval, PR interval, QRS interval, QT interval, and corrected QT interval, will include mean, standard deviation, median, and range for each assessment time point, as well as for the calculated changes from baseline to each subsequent time point. The categorical data, ie, diagnoses, will be summarized using a shift table from baseline to each subsequent assessment time point and categories of normal, abnormal NCS, and abnormal CS.

Tabular summaries of safety laboratory tests will include descriptive statistics (ie, mean, standard deviation, median, and range for continuous data and frequency for categorical data) for each assessment time point, and for the calculated changes from baseline to each subsequent assessment time point. Summaries of the safety laboratory tests relative to laboratory reference ranges will be prepared using shift tables from baseline to each subsequent assessment time point and categories of low, normal, and high for continuous data and abnormal and normal for categorical data.

The incidence of treatment-emergent abnormal laboratory, vital sign, and ECG values will also be summarized using descriptive statistics.

### 9.4.2 Efficacy Analyses

Efficacy endpoints will be analyzed for the full efficacy analysis set as described in Table 14. All proportions will be estimated with 95% confidence intervals.

TABLE 14
Efficacy Analyses

| Endpoint | Statistical Analysis Methods |
|---|---|
| Secondary | Estimate the proportion of patients with different levels of overall response |
| | for their primary tumor and any metastases will be determined at the end of |
| | induction therapy for each cohort (cycle 6 for GPOH and cycle 7 for COJEC) |
| | and each cumulative dose level within each cohort. |
| Secondary | Estimate the proportion of patients achieving metastatic complete response |
| | after 2 cycles (GPOH) and after 4 cycles (COJEC) and at the end of induction |
| | therapy for each cohort. |
| Secondary | Estimate the proportion of patients achieving metastatic partial response (see |
| | definition under endpoints in Section 3) at the end of induction therapy for |
| | each cohort. |
| Secondary | Estimate cumulative incidence, median survival time and proportion of |
| | patients surviving at end-of-study when considering only treatment-related |
| | mortality. |
| Secondary | Estimate cumulative incidence, median survival time and proportion of |
| | patients surviving at end-of-study when considering only disease-related |
| | mortality. |
| Exploratory | Estimate Kaplan-Meier curve for 3- and 5-year EFS (see definition under |
| | endpoints). |
| Exploratory | Estimate Kaplan-Meier curve for 3- and 5-year OS (see definition under |
| | endpoints in Section 3). |

EFS = event-free survival; GPOH = German Pediatric Oncology and Hematology; COJEC = cisplatin, vincristine, carboplatin, etoposide, and cyclophosphamide A vincristine, carboplatin, etoposide; B vincristine, cisplatin; C vincristine, cyclophosphamide, etoposide; OS = overall survival.

### 9.4.3 Other Analyses

Baseline demographics, medical conditions, and performance status will be summarized.

Exploratory PK, pharmacodynamic, immunogenicity, and biomarker analyses will be further described in the SAP finalized before database lock.

Serum concentrations for dinutuximab beta will be listed and will be summarized by chemotherapy induction regimen, cumulative dinutuximab beta in the cycle, and nominal time point using descriptive statistics. The proportion of patients who achieve a dinutuximab beta drug concentration level >1 μg/mL at the end-of-infusion of the first combination cycle will be tabulated.

### 9.4.4 Missing Data

Missing or incomplete data will not be imputed unless indicated otherwise in the SAP.

### 9.5 Interim Analyses

No Interim analysis for efficacy is planned in this study.

Safety analysis and tolerability will be reviewed by the DMC on an ongoing basis to determine dose escalation and de-escalation (Section 6.7.3).

### 10. References

1. Peuchmaur M, d'Amore E S, Joshi V V, Hata J, Roald B, Dehner L P, et al. Revision of the International Neuroblastoma Pathology Classification: confirmation of favorable and unfavorable prognostic subsets in ganglioneuroblastoma, nodular. Cancer. 2003; 98(10):2274-81.
2. Lode H N, Valteau-Couanet D, Garaventa A, Gray J, Castel V, Yaniv I, et al. Long-term infusion (LTI) of anti-GD2 antibody ch14.18/CHO may improve outcome in patients (pts) with high-risk relapsed/refractory neuroblastoma (NB). J Clin Oncol. 2015; 33:(suppl; abstr TPS10080).
3. Mody R, Naranjo A, Van Ryn C, Yu A L, London W B, Shulkin B L, et al. Irinotecan-temozolomide with temsirolimus or dinutuximab in children with refractory or relapsed neuroblastoma (COG ANBL1221): an open-label, randomised, phase 2 trial. Lancet Oncol. 2017.
4. Dinutuximab Beta Investigator's Brochure. Version 3.0 dated 14 May 2019
5. Ladenstein R, Potschger U, Hartman O, Pearson A D, Klingebiel T, Castel V, et al. 28 years of high dose therapy and SCT for neuroblastoma in Europe: lessons from more than 4000 procedures. Bone Marrow Transplant. 2008; 41 Suppl 2:S118-27.
6. Berthold F, Boos J, Burdach S, Erttmann R, Henze G, Hermann J, et al. Myeloablative megatherapy with autologous stem cell rescue versus oral maintenance chemotherapy as consolidation treatment in patients with high-risk neuroblastoma: a randomised controlled trial. Lancet Oncol. 2005; 6(9):649-58.
7. Simon T, Hero B, Schulte J H, Deubzer H, Hundsdoerfer P, von Schweinitz D, et al. 2017 GPOH Guidelines for Diagnosis and Treatment of Patients with Neuroblastic Tumors. Kiln Padlatr. 2017; 229(3):147-67.
8. Cheung N K, Kushner B H, LaQuaglia M, Kramer K, Gollamudi S, Heller G, et al. N7: a novel multi-modality therapy of high-risk neuroblastoma (NB) in children diagnosed over 1 year of age. Med Pediatr Oncol. 2001; 36(1):227-30.
9. Kushner B H, Kramer K, LaQuaglia M P, Modak S, Yataghene K, Cheung N K. Reduction from seven to five cycles of intensive Induction chemotherapy in children with high-risk neuroblastoma. J Clin Oncol. 2004; 22(24):4888-92.
10. Mora J, Cruz O, Lavarino C, Rios J, Vancells M, Parareda A, et al. Results of induction chemotherapy in children older than 18 months with stage-4 neuroblastoma treated with an adaptive-to-response modified N7 protocol (mN7). Clin Transl Oncol. 2015; 17(7):521-9.
11. Furman W L, Federico S M, McCarville M B, Shulkin B L, Davidoff A M, Krasin M J, et al. A Phase I I trial of Hu14.18K322A In combination with Induction chemotherapy in children with newly diagnosed high-risk neuroblastoma. Clin Cancer Res. 2019; 25(21):6320-6328.
12. Siebert N, Eger C, Seidel D, et al. Pharmacokinetics and pharmacodynamics of ch14.18/CHO in relapsed/refractory high-risk neuroblastoma patients treated by long-term infusion in combination with IL-2. MAbs. 2016; 8(3):604-616.
13. Lode H N, Jensen C, Endres S, et al. Immune activation and clinical responses following long-term infusion of anti-GD2 antibody ch14.18/CHO in combination with interleukin-2 in high-risk neuroblastoma patients. Journal of Clinical Oncology. 2014; 32:5.
14. Bodnar L, Wcislo G, Gasowska-Bodnar A, Synowiec A, Szarlej-Wcislo K, Szczylik C. Renal protection with magnesium subcarbonate and magnesium sulphate in patients with epithelial ovarian cancer after cisplatin and paclitaxel chemotherapy: a randomised phase II study. Eur J Cancer. 2008; 44(17):2608-14.
15. Hunter R I, Pace M B, Burns K A, Burke C C, Gonzales D A, Webb N F, et al. Evaluation of intervention to prevent hypomagnesemia in cervical cancer patients receiving combination cisplatin and radiation treatment. Support Care Cancer. 2009; 17(9):1195-201.
16. van Angelen A A, Glaudemans B, van der Kemp A W, Hoenderop J G, Bindels R J. Cisplatin-induced injury of the renal distal convoluted tubule is associated with hypomagnesemia in mice. Nephrol Dial Transplant. 2013; 28(4):879-89.
17. Yoshida T, Niho S, Toda M, Goto K, Yoh K, Umemura S, et al. Protective effect of magnesium preloading on cisplatin-induced nephrotoxicity: a retrospective study. Jpn J Clin Oncol. 2014; 44(4):346-54.
18. Park J R, Bagatell R, Cohn S L, Pearson A D, Villablanca J G, Berthold F, et al. Revisions to the International Neuroblastoma Response Criteria: A Consensus Statement From the National Cancer Institute Clinical Trials Planning Meeting. J Clin Oncol. 2017; 35(22):2580-7.
19. Fellman B M, Yuan Y. Bayesian optimal interval design for phase I oncology clinical trials. The Stata Journal. 2015; 15(1):110-120.
20. Ying Y, Kenneth R H, Susan G H, Mark R G. Bayesian optimal interval design: a simple and well-performing design for phase I oncology trials. Clin Cancer Res. 2016; 22(17):4291-4301. doi:10.1158/1078-0432.CCR-16-0592.

### Appendices

APPENDIX 1
Abbreviations

| Abbreviation | Definition |
|---|---|
| AE | Adverse event |
| ALT | Alanine aminotransferase |
| ANC | Absolute neutrophil count |
| AST | Aspartate aminotransferase |
| ASCT | Autologous stem cell transplant |
| BOIN | Bayesian optimal interval |
| CDC | Complement-dependent cytotoxicity |
| COJEC | Cisplatin, vincristine, carboplatin, etoposide, and |
| | cyclophosphamide |
| CRF | Case report form |
| CRO | Contract research organization |
| CR | Complete response |
| CS | Clinically significant |
| CRP | C-reactive protein |
| CT | Computed tomography |
| CTCAE | Common Terminology Criteria for Adverse Events |
| Cycle A | Vincristine, carboplatin, etoposide |
| Cycle B | Vincristine, cisplatin |
| Cycle C | Vincristine, cyclophosphamide, etoposide |
| Cycle N5 | Cisplatin, etoposide, vindesine |
| Cycle N6 | Vincristine, dacarbazine, ifosfamide, doxorubicin |
| DMC | Data monitoring committee |
| DLT | Dose-limiting toxicity |
| ECG | Electrocardiogram |
| eCRF | Electronic case report form |
| EDTA | Ethylenediaminetetraacetic acid |
| EFS | Event-free survival |
| EU | European Union |
| FSH | Follicle stimulating hormone |
| GCP | Good clinical practice |
| GFR | Glomerular filtration rate |
| GPOH | German Pediatric Oncology and Hematology |
| G-CSF | Granulocyte-colony stimulating factor |
| GvHD | Graft versus host disease |
| HACA | Human anti-chimeric antibody |
| HDC | High-dose chemotherapy |
| HIV | Human immunodeficiency virus |
| HRT | Hormonal replacement therapy |
| ¹²³I-MIBG | ¹²³I metaiodobenzylguanidine |
| ICF | Informed consent form |
| ICH | International Council for Harmonisation |
| IEC | Independent Ethics Committee |
| INPC | International Neuroblastoma Pathology Classification |
| INRC | International Neuroblastoma Response Criteria |
| INRGSS | International Neuroblastoma Risk Group Staging System |
| IRB | Institutional Review Board |
| LDH | Lactate dehydrogenase |
| LTI | Long-term infusion |
| MedDRA | Medical Dictionary for Regulatory Activities |
| MR | Minor response |
| MRI | Magnetic resonance imaging |
| MTD | Maximum tolerated dose |
| NCI | National Cancer Institute |
| NCS | Not clinically significant |
| OS | Overall survival |
| PFT | Pulmonary function test |
| PK | Pharmacokinetics |
| PR | Partial response |
| PT | Preferred term |
| RP2D | Recommended Phase 2 dose |
| SAE | Serious adverse event |
| SAP | Statistical analysis plan |
| SD | Stable disease |
| SIOPEN | International Society of Paediatric Oncology European |
| | Neuroblastoma research network |
| SoA | Schedule of assessments |
| SOC | System organ class |
| SOP | Standard operating procedure |
| SPECT | Single-photon emission tomography |
| ULN | Upper limit of normal |

### Appendix 2 Excluded Medications/Therapy

Excluded medications/therapy are listed below. The use of an excluded medication/therapy is a protocol violation and must be recorded in the eCRF.
Due to immunosuppressive activity, the concomitant treatment with corticosteroids is not allowed within 2 weeks prior to the first treatment course and until 1 week after the last treatment course with dinutuximab beta, except for life-threatening conditions.
Vaccinations (including seasonal influenza) will not be allowed during administration of dinutuximab beta until 10 weeks after last treatment course, due to immune stimulation through dinutuximab beta and possible risk for rare neurological toxicities.
Concomitant use of intravenous (iv) Immunoglobulins will not be allowed as they may interfere with dinutuximab beta-dependent cellular cytotoxicity.
The cardio protectant dexrazoxane will not be administered.
Any systemic anticancer treatment not prescribed in this protocol. Emergency radiation for life-threatening or function threatening (eg, tracheal compression) disease prior to start of treatment will be allowed.
Tumor directed surgery outside of protocol-scheduled surgery will not be allowed.

### Appendix 3 Instructions for the Administration of Concomitant Medications

### Gabapentin

Prior to each infusion cycle of dinutuximab beta, the patient should be primed with oral gabapentin starting 3 days before the start of the continuous dinutuximab beta infusion. The recommended oral dose of gabapentin is 10 mg/kg/dose once daily on Day 3 before the start of dinutuximab beta administration, increasing to 10 mg/kg/dose twice daily on Day 2 before the start of dinutuximab beta administration and 10 mg/kg/dose 3 times a day thereafter on all subsequent days during the antibody infusion, if required by the patient. Oral gabapentin shall be tapered off latest after stop of the dinutuximab beta infusion in a reverse order of the priming schedule.

### Morphine

To prevent severe visceral and neuropathic pain opioids are the standard pain treatment given with dinutuximab beta. The first day and cycle usually requires more than subsequent days and cycles. Opioids should therefore be started and then gradually weaned. Besides respiratory depression and sedation, the same adverse effects are essentially seen in children as in adults (nausea, vomiting, constipation, pruritus, urinary retention, lowered seizure threshold). The safety and efficacy of continuous Iv administration of opioids for pain management are well established for all age groups. The risk of dependence is classified as low.

During the first cycle iv morphine should initially be given, eg, a morphine sulfate loading infusion (30 μg/kg/h) in 60 minutes prior to the start of the continuous infusion of dinutuximab beta. Thereafter, it is recommended to administer morphine sulfate at a continuous infusion rate of 30 μg/kg/h on the first day. Boluses can be given as required. It is expected that the iv morphine can be rapidly tapered off. Depending on the individual patient's pain tolerance, subsequent cycles can be started with iv morphine, including the bolus loading dose, and tapered at the discretion of the treating team.

Other opioids are allowed in the tapering phase and after continuous morphine infusion:
Oral morphine; administered at a dose of 0.2 to 0.4 mg/kg every 4 to 6 hours.
Oral tramadol; if pain is well-controlled on low doses of oral morphine.

The equivalent morphine to transdermal fentanyl dose rate in μg/h will be calculated from the current use of iv morphine, according to the manufacturer's guidance, and the dose gradually decreased according to pain symptoms. It is not advised to use long-acting morphine in this situation, as it takes 48 hours to stabilize the dose, and this is not practical.

### Non-Steroidal Anti-Inflammatory Drugs

In the first cycle, to prevent febrile reactions and to support pain prophylaxis, one of the antipyretic drugs listed below should be used during antibody infusion. In subsequent cycles antipyretic drugs should be administered at the investigators discretion or according to local guidelines.
Metamizol (ie, Novalgin®) 10 to 15 mg/kg po every 6 to 8 hours, if needed, or iv LTI with a dosage of 2.5 to 3.0 mg/kg/h.
Paracetamol 15 mg/kg po every 6 hours, if needed, or iv (ie, Perfalgan®) every 6 hours 15 mg/kg (100 mg/100 mL).
Ibuprofen (eg, Ibuprofen-ratiopharm 2% or 4% Syrup®, Thomapyrin®) 10 to 15 mg/kg po before the treatment is administered and every 8 hours, if needed.

### Other Supportive Care Measures

For anticipated potential of dinutuximab beta for allergic reactions, antihistamines as per local policy are recommended. For example:
Diphenhydramine (ie, DIBONDRIN®) 0.5 to 1.0 mg/kg iv/po q4h prn.
Cetirizine: 2 to 6 years: 2.5 mg/12 h po; 6 to 12 years: 5 mg/12 h po; >12 years: 10 mg/24 h po.
Dimetindenmaleat (FENISTIL®): 0.1 mg/kg iv q4h prn.
Chlorphenamine: dose as per local guidelines.
Hidroxicin (Atarax®): 0.5 mg/kg po q 8 h prn.

For the potential side effects of non-selective non-steroidal anti-Inflammatory drugs as cyclooxygenase (COX) type I and II inhibitors and its effects on platelet aggregation (increased hemorrhagic risk, gastrointestinal mucosal injury): Proton pump inhibitors or H2-receptor antagonists according to institutional use need to be considered. For example:
Omeprazole 1 mg/kg/day po or iv.

### Hydration

Minimum hydration during dinutuximab beta is 1500 mL/m² and can be given iv or orally.

### Appendix 4 Specific Product-Related Toxicities: Guidance on Early Detection and Management

### Constitutional Symptoms and Fever

Temperature elevations >38° C. should be treated with appropriate doses of antipyretics according to institutional standards. Persistent temperature elevations >38° C. which are causing the patient's symptoms may also be treated with a cooling blanket.

### Blood Cultures Are Recommended to Exclude Septicemia.

Other antipyretic schedules established as institutional standards are allowed. No dose modifications will be made for temperature elevations, unless the temperature exceeds >40° C. and persists more than 6 hours despite antipyretics, in which case dinutuximab beta will be withheld.

If Grade 4 constitutional symptoms or fevers (>40.0° C. >24 hours) occur while the patient is getting dinutuximab beta despite adequate measures and resolve to Grade 51 or to baseline, administration of the dinutuximab beta may be resumed for subsequent therapy.

Grade 3 fevers (>40.0° C. <24 hours) will NOT need dose modification when observed, provided that these toxicities are judged to be tolerable by the responsible clinician.

In case fever occurs in the context of neutropenia of any cause (disease or chemotherapy associated), treatment with antibiotics according to institutional guidelines is indicated. Dinutuximab beta infusion continues providing patient is otherwise clinically stable.

### Hypotension

For Grade 3 (medical intervention indicated) or Grade 4 (life-threatening) not responsive to a 20 mL/kg 0.9% sodium chloride fluid challenge.

Dose Modifications During Current Course of immunotherapy:
Discontinue dinutuximab beta Infusion, support blood pressure with iv fluids, and with vasopressors if necessary.
If hypotension resolves or improves to Grade 1 with fluid boluses, then dinutuximab beta treatment may resume at 50% rate 1 hour later. To complete the administration of the prescribed dose prolongation of the infusion time period is possible according to the 20% rule.
If hypotension requires treatment with inotropic support, then discontinue dinutuximab beta and the patient will be taken off protocol therapy.

If hypotension is unresponsive to supportive measures or requires ventilator support, the patient will be discontinued from dinutuximab beta.

Hyponatremia Associated with Dinutuximab Beta

Patients with either symptomatic hyponatremia, persistent (>48 hours) sodium less than 130 mmol/L, or severe hyponatremia without symptoms (sodium less than 120 mmol/L) will be discontinued from study therapy and receive no further dinutuximab beta.

Grade 3 electrolyte imbalance (especially hyponatremia <130 mmol/L in the absence of central nervous system (CNS) symptoms and sequelae) that improves with treatment within 24 hours will NOT require dose modification when observed, provided that these toxicities are judged to be tolerable by the responsible clinician, as well as the patient and family.

### Performance Status

Treatment will be stopped for a performance status <20%. If performance status improves to ≥20%, dinutuximab beta treatment can be restarted at 50% of the dose, at which this toxicity occurred.

Performance status (30% to <50%) will NOT need dose modification when observed, provided that these toxicities are judged to be tolerable by the responsible clinician, as well as the patient and family.

### Pain

Patients experiencing pain due to dinutuximab beta despite pain prophylaxis (Section 6.6) will be treated with additional morphine or similar analgesics, as needed, and have their pain graded according to the NCI-CTCAE version 5.0 and, in addition, will be evaluated with a validated self-reporting tool (Appendix 5).

Patients with severe unrelenting neuropathic pain unresponsive to continuous infusion of narcotics and other adjuvant measures including lidocaine infusions must be discontinued from study therapy and the Sponsor contacted if the patient may continue the study.

### Hematologic Toxicity

All patients will be transfused as needed to maintain an adequate hemoglobin level and platelet count. If the patient experiences neutropenia while receiving dinutuximab beta, treatment would not be interrupted. Transfusions are allowed also during dinutuximab beta therapy. Grade 3 hematologic toxicity does NOT require dose modification of dinutuximab beta. Treatment start are defined by the rules applicable to the various induction chemotherapy regimen (Section 0).

### Hepatic Toxicity

Grade 3 hepatic toxicity that has been present for <7 days or returns to Grade 1 prior to the time for next treatment course, will NOT need dose modification when observed, provided

that these toxicities are judged to be tolerable by the responsible clinician, as well as the patient and family.

Transaminases (ALT and AST): In the event of persistent clinically relevant elevation of transaminase levels (Grade 4, >7 days), discontinuation of treatment should be considered.

Alkaline phosphatase: No dosing interruption or dose modifications will be made for elevated alkaline phosphatase since this occurs commonly and, on its own, is not a good indicator of hepatic toxicity.

Bilirubin: If total bilirubin increases to Grade 3 (>3×ULN) due to chemotherapy/ch.14.18/CHO toxicity, the dinutuximab beta antibody should be withheld until the total bilirubin returns to normal. Following recovery, the dinutuximab beta antibody should be restarted at the planned dose.

### Nephrotoxicity

Adequate renal function is an eligibility requirement. If renal function is worsening, but not yet Grade 3 (creatinine >3×baseline), other nephrotoxic drugs should be avoided.

### Cardiac Toxicity

Any evidence of cardiac abnormalities will require an immediate ECG evaluation. Evidence of ischemia will require immediate discontinuation of therapy. Patients with evidence of asymptomatic atrial irregularities, related to an elevated temperature, but without any evidence of ischemia or clinically significant hypotension, will be monitored but continue therapy.

Patients experiencing Grade ≥3 cardiac toxicity will be taken off protocol therapy. Complications of fluid overload may be seen. Patients with clinical problems related to fluid overload will be treated with furosemide or mannitol provided they have <40 mmHg decrease in systolic blood pressure from baseline.

Treatment should be stopped for a sustained decrease in blood pressure below 40 mmHg mean arterial blood pressure. This is also the case if blood pressure has not been restored with brief fluid or albumin challenge, ie, 20 mL/kg of 0.9% sodium chloride. Intravenous vasopressor may be used when clinically indicated. Treatment can be restarted at 50% of the dose of the dinutuximab beta that caused the toxicity, once the blood pressure has returned to 40 mmHg mean arterial pressure.

### Dyspnea

Patients experiencing dyspnea and whose oxygen saturation is less than 90% may receive oxygen supplementation. Patients with clinical problems related to fluid overload will be treated with diuretics provided they have <40 mmHg decrease in systolic blood pressure from baseline. If the oxygen saturation does then not improve to over 90%, treatment will be discontinued and restarted at 50% of the previous dose of the dinutuximab beta dose when toxicity has been resolved.

### Neurotoxicity

Neuropathy associated with dinutuximab beta.

Occasional reports of neuropathy with weakness and paralysis and MRI signs of transverse myelitis have been reported mostly with dinutuximab beta given in combination with IL-2. Although dinutuximab beta is not being used in combination with IL-2 the possibility of occurrence of this severe neurotoxicity cannot be excluded. Patient experiencing transverse myelitis are to be taken off protocol and should receive steroids, iv immunoglobulins, and possibly plasmapheresis.

Patients experiencing Grade 2:3 neurotoxicity except confusion as noted below will be discontinued from study therapy.

### Confusion

Confusion which is clearly not temperature related or related to supportive care medicines (diphenhydramine, morphine, etc.) will result in the interruption of treatment. Confusion related to fever will be managed by adapted use of antipyretics and cooling blankets.

Persistent confusion (>6 hours) of any cause will require the discontinuation of therapy, with subsequent reinitiating of treatment at 50% of the previous dose if reversal of this toxicity occurs within 5 days prior to the next scheduled dose.

### Pruritus and Urticaria

Treat with antihistamines (eg, diphenhydramine or chlorpheniramine). Preparation of emergency medication (corticosteroids and epinephrine) as per local resuscitation guidelines in case severe anaphylactic reaction occurs. A free-flowing iv line must be established at all times.

### Nausea, Vomiting, and Diarrhea

Grade 3 nausea, vomiting and diarrhea will NOT need dose modification. Use of anti-emetics is will be done according to institutional guidelines, avoiding the concomitant use of corticosteroids. In case of diarrhea treatment with loperamide could be considered once microbial etiology has been ruled out (viral, bacterial, Clostridium difficile).

### Skin Toxicity

Grade 3 skin toxicity that improves with treatment (eg, iv diphenhydramine or chlorphenamine) will NOT need dose modification. Grade 4 skin toxicity patients should be taken off dinutuximab beta.

### Impaired Vision

Impaired visual accommodation, correctable with spectacles will NOT need dose modification. Tinted spectacles are recommended for mydriasis associated sensitivity to light.

### Altered Taste

Altered taste will not require dose modification.

### Hypersensitivity Reactions

### Mild Symptoms (Eg, Localized Cutaneous Reactions or Shivering and Rigor)

Decrease the rate of dinutuximab beta infusion to 50% until recovery from symptoms, remain at bedside and monitor patient; complete infusion at the initial planned rate. Antihistamines (diphenhydramine or chlorphenamine) may be administered every 4 to 6 hours at the discretion of the treating physician.

### For Moderate Symptoms (eg, Hypotension)

Interrupt dinutuximab beta infusion, administer supportive care, and monitor patient until resolution of symptoms. At the discretion of the treating physician, infusion may be resumed at 50% of the initial infusion rate.

### For Severe Symptoms (any Reaction Such as Bronchospasm, Angioedema or Anaphylactic Shock)

Immediately discontinue infusion. Give epinephrine, antihistamines (diphenhydramine or chlorphenamine) and corticosteroids, bronchodilator or other medical measures as needed. Patients should be monitored as inpatients for at least 24 hours AND until the symptoms have resolved.
Patients should be closely monitored for anaphylaxis and allergic reactions, particularly during Cycles 1 and 2 of treatment. Patients who experience Grade 4 anaphylaxis or allergic reaction should discontinue dinutuximab beta therapy.
Grade 3 serum sickness patients should be taken off dinutuximab beta.

### Capillary Leak and Cytokine Release Syndrome

Dose modification instructions during the course of immunotherapy for Grade 3 syndrome:
Discontinue dinutuximab beta Infusion.
If syndrome resolves or improves to Grade 2 with supportive measures, dinutuximab beta may resume at 50% rate 1 hour later to complete the prescribed dose.
If syndrome (Grade 3) recurs with dinutuximab beta discontinue immunotherapy.
If syndrome (Grade 4) includes ventilator support, patients should be taken off study.

In other immunotherapy studies using engineered T-cells to express chimeric antigen receptors (CAR T-cells), tocilizumab was described to be effective against cytokine release syndrome and may be considered for severe cases of cytokine release syndrome.

### Dose Modification Instructions for Subsequent Courses:

If patient tolerated, subsequent course of dinutuximab beta should start at the last tolerated infusion rate in the previous course. If tolerated, advancing dinutuximab beta Infusion to full rate may be considered.
If capillary leak syndrome is unresponsive to supportive measures or requires ventilator support, patient will be discontinued from dinutuximab beta.

### Grade 3 Infection During Infusion of Dinutuximab Beta

Abort the dinutuximab beta treatment course. Missed doses will not be replaced. Patient may proceed to the subsequent planned course only when infection resolves or is under control (asymptomatic and negative blood culture).

APPENDIX 5
Pain Assessment Scores

| | | | Ramsay | | | | |
|---|---|---|---|---|---|---|---|
| | | | Score to | | | | |
| Kindliche Unbehagenund | | | Evaluate | | Modification of the | | |
| Schmerz-Skala: | | | Sedated | | Objective Pain Score | | |
| Patients <4 years of age | | | Patients | | Patients: >4 years of age | | |
| Crying | None | 0 | 1 | Anxious, | Crying | None | 0 |
| | | | | agitated, | | | |
| | | | | restless | | | |
| | Groaning, | 1 | 2 | Cooperative, | | Consolable | 1 |
| | moaning, | | | tranquil, | | | |
| | whining | | | oriented | | | |
| | Screaming | 2 | 3 | Drowsy, | | Not | 2 |
| | | | | response | | consolable | |
| | | | | to verbal | | | |
| | | | | command | | | |
| Facial | Smiling, | 0 | 4 | Asleep, | Movement | None | 0 |
| Expression | relaxed | | | brisk | | | |
| | | | | response | | | |
| | | | | to light | | | |
| | | | | glabellar | | | |
| | | | | tap and | | | |
| | | | | loud | | | |
| | | | | auditory | | | |
| | | | | stimulus | | | |
| | Distorted | 1 | 5 | Asleep; | | Restless | 1 |
| | mouth | | | sluggish | | | |
| | | | | response | | | |
| | | | | to light | | | |
| | | | | glabellar | | | |
| | | | | tap and | | | |
| | | | | loud | | | |
| | | | | auditory | | | |
| | | | | stimulus | | | |
| | Face | 2 | 6 | Coma | | Thrashing | 2 |
| | distorted, | | | | | | |
| | grimace | | | | | | |
| Bearing of | Neutral | 0 | | | Agitation | Asleep | 0 |
| the Torso | | | | | | | |
| | Erratic | 1 | | | | Calm | 0 |
| | Rear up, | 2 | | | | Mild | 1 |
| | convulse | | | | | | |
| Bearing of | Neutral | 0 | | | | Hysterical | 2 |
| legs | | | | | | | |
| | Struggling, | 1 | | | Posture | Normal | 0 |
| | kicking | | | | | | |
| | Dragged to | 2 | | | | Flexed | 1 |
| | the body | | | | | | |
| Motoric | Not | 0 | | | | Holds | 2 |
| restlessness | present | | | | | injury site | |
| | Moderate | 1 | | | Verbal | Asleep | 0 |
| | Restless | 2 | | | | No | 0 |
| | | | | | | complaint | |
| | | | | | | Complains | 1 |
| | | | | | | but cannot | |
| | | | | | | localize | |
| | | | | | | Complains | 2 |
| | | | | | | and can | |
| | | | | | | localize | |

### References

Buttner W, Breitkopf L, Miele B, Finke W. (Initial results of the reliability and validity of a German-language scale for the quantitative measurement of postoperative pain in young children]. Anaesthesist. 1990; 39(11):593-602.
Ramsay M A, Savege T M, Simpson B R, Goodwin R. Controlled sedation with alphaxalone-alphadolone. Br Med J. 1974; 2(5920):656-9.
Zernikow B, Hechier T. Pain therapy in children and adolescents. Dtsch Arztebl Int. 2008; 105(28-29):511-21.

APPENDIX 6
International Neuroblastoma Response Criteria
See reference 18 for more information.
Primary (soft tissue) Tumor Response

| Response | Anatomic + MIBG (FDG-PET†) Imaging |
|---|---|
| CR | <10 mm residual soft tissue at primary site AND |
| | Complete resolution of MIBG or FDG-PET uptake (for MIBG- |
| | nonavid tumors) at primary site |
| PR | ≥30% decrease in longest diameter of primary site AND |
| | MIBG or FDG-PET uptake at primary site stable, improved, or |
| | resolved |
| PD | >20% increase in longest diameter taking as reference the |
| | smallest sum on study (this includes the baseline sum if that |
| | is the smallest on study) AND |
| | Minimum absolute increase of 5 mm in longest dimension^{‡} |
| SD | Neither sufficient shrinkage for PR nor sufficient increase for PD |
| | at the primary site |

Abbreviations: CR, complete response; FDG, [¹⁸F] fluorodeoxyglucose;
MIBG, metalodobenzylguanidine; PD, progressive disease; PET,
positron emission tomography; PR, partial response; SD, stable disease.
*Not for use in assessment of metastatic sites.
†Used for MIBG-nonavid tumors.
^{‡}Mass that does not meet PD measurement criteria
but has fluctuating MIBG avidity will not be considered PD.

Tumor Response at Metastatic Soft Tissue and Bone Sites

| Response | Anatomic + MIBG (FDG-PET*) Imaging |
|---|---|
| CR | Resolution of all sites of disease, defined as: |
| | Nonprimary target and nontarget lesions measure <10 mm |
| | AND |
| | Lymph nodes identified as target lesions decrease to a short |
| | axis <10 mm AND |
| | MIGB uptake or FDG-PET uptake (for MIBG-nonavid tumors) of |
| | nonprimary lesions resolves completely |
| PR | ≥30% decrease in sum of diameterst of nonprimary target |
| | lesions compared with baseline AND all of the following: |
| | Nontarget lesions may be stable or smaller in size AND |
| | No new lesions AND |
| | ≥50% reduction in MIBG absolute bone score (relative MIBG |
| | bone score ≥0.1 to ≤0.5) or ≥50% reduction in number of |
| | FDG-PET-avid bone lesions^{‡}§ |
| PD | Any of the following: |
| | Any new soft tissue lesion detected by CT/MRI that is also |
| | MIBG avid or PDG-PET avid |
| | Any new soft tissue lesion seen on anatomic imaging that is |
| | biopsied and confirmed to be neuroblastoma or |
| | ganglioneuroblastoma |
| | Any new bone site that is MIBG avid |
| | A new bone site that is FDG-PET avid (for MIBG-nonavid |
| | tumors) AND has CT/MRI findings consistent with tumor OR |
| | has been confirmed histologically to be neuroblastoma or |
| | ganglioneuroblastoma |
| | >20% increase in longest diameter taking as reference the |
| | smallest sum on study (this includes the baseline sum if that |
| | is the smallest on study) AND minimum absolute increase of |
| | 5 mm in sum of diameters of target soft tissue lesions |
| | Relative MIBG score ≥1.2§ |
| SD | Neither sufficient shrinkage for PR nor sufficient increase for |
| | PD of nonprimary lesions |

Abbreviations: CR, complete response; CT, computed tomography; FDG, [¹⁸F]
fluorodeoxyglucose; MIBG, metalodobenzylguanidine; MRI, magnetic resonance
imaging; PD, progressive disease; PET, positron emission tomography; PR, partial
response; SD, stable disease.
*Used for MIBG-nonavid tumors
†Sum of diameters is defined as the sum of the short axis of discrete lymph nodes (ie,
cervical, axillary nodes) added to the sum of the longest diameters of non-lymph node
soft tissue metastases. Masses of conglomerate nondiscrete lymph nodes will be
measured using longest diameter.
^{‡}For patients with soft tissue metastatic disease, resolution of MIBG and/or PDG-PET
uptake at the soft tissue sites is not required; all size reduction criteria must be fulfilled.
§Relative MIBG score is the absolute score for bone lesions at time of response
assessment divided by the absolute score for bone lesions at baseline before
therapeutic interventions. The same scoring method (eg. Curie or International Society
of Pediatric Oncology European Neuroblastoma) must be used at all assessment time
points. MIBG single-photon emission computed tomography (SPECT) or MIBG-
SPECT/CT may be used for scoring purposes, but the same imaging methodology
should be used for all evaluations.

Determination of Overall Response

| Response | Cytology^{†}/Histology^{‡} |
|---|---|
| CR | Bone marrow with no tumor infiltration on reassessment, |
| | independent of baseline tumor involvement |
| PD | Any of the following: |
| | Bone marrow without tumor infiltration that becomes >5% |
| | tumor infiltration on reassessment OR |
| | Bone marrow with tumor infiltration that increases by >two- |
| | fold and has >20% tumor infiltration on reassessment |
| MD | Any of the following: |
| | Bone marrow with ≤5% tumor infiltration and remains >0 |
| | to ≤5% tumor infiltration on reassessment OR |
| | Bone marrow with no tumor infiltration that |
| | has ≤5% tumor infiltration on reassessment OR |
| | Bone marrow with >20% tumor infiltration that has >0 |
| | to ≤5% tumor infiltration on reassessment |
| SD | Bone marrow with tumor infiltration that remains positive |
| | with >5% tumor infiltration on reassessment but does not meet |
| | CR, MD, or PD criteria |

NOTE.
In the case of discrepant results between aspirations or core biopsies from two
or more sites taken at the same time, the highest infiltration result should be reported
using the criteria in this table.
Abbreviations: CR, complete response; MD, minimal disease; PD, progressive disease;
SD, stable disease.
*Response will be compared with baseline disease evaluations before enrollment in a
clinical trial or, for newly diagnosed patients, with baseline at specific times during
therapy (ie, at diagnosis and before start of therapy, before specific phases of therapy
such as induction, high-dose chemotherapy with stem-cell rescue consolidation, or
postconsolidation immunotherapy).
^{†}Accompanied by immunocytology (recommended, not mandatory).
^{‡}Accompanied by immunohistochemistry; specific recommendations included in article
by Burchill et al (19).

1. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a cumulative dose of up to 500 mg/m².

2. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient in a dose of up to 100 mg/m² per cycle during one or more cycles of induction chemotherapy.

3. The method of claim 2, wherein the chimeric anti-GD2 antibody dinutuximab beta is administered to the patient during the induction chemotherapy in a dose per cycle that is equal for all cycles during which the chimeric anti-GD2 antibody dinutuximab beta is administered.

4. The method of claim 2, wherein the chimeric anti-GD2 antibody dinutuximab beta is administered to the patient during the induction chemotherapy in a dose per cycle that varies by up to 20 mg/m² between different cycles during which the chimeric anti-GD2 antibody dinutuximab beta is administered.

5. The method of claim 2, wherein the chimeric anti-GD2 antibody dinutuximab beta is administered to the patient during the induction chemotherapy in a dose per cycle of 20 mg/m² to 100 mg/m² during one or more cycles of induction chemotherapy.

6. The method of claim 1, wherein the chimeric anti-GD2 antibody dinutuximab beta is administered to the patient during the induction chemotherapy in a dose of up to 100 mg/m² per cycle during one or more cycles of induction chemotherapy.

7. The method of claim 3, wherein the chimeric anti-GD2 antibody dinutuximab beta is administered to the patient during the induction chemotherapy at a cumulative dose of up to 500 mg/m².

8. A method of treating a newly diagnosed neuroblastoma in a patient by administering chimeric anti-GD2 antibody dinutuximab beta to the patient in combination with induction chemotherapy, wherein the chimeric anti-GD2 antibody dinutuximab beta is administered to the patient during the induction chemotherapy at a treatment density of up to 5 mg/m²/day, wherein a newly diagnosed neuroblastoma is treated in the patient.

9. The method of claim 8, wherein the chimeric anti-GD2 antibody dinutuximab beta is administered to the patient during the induction chemotherapy at a treatment density of at least 1 mg/m²/day.

10. The method of claim 1, wherein the chimeric anti-GD2 antibody dinutuximab beta is administered to the patient during the induction chemotherapy at a treatment density of up to 5 mg/m²/day.

11. The method of claim 2, wherein the chimeric anti-GD2 antibody dinutuximab beta is administered to the patient during the induction chemotherapy at a treatment density of up to 5 mg/m²/day.

12. The method of claim 9, wherein the chimeric anti-GD2 antibody dinutuximab beta is administered to the patient during the induction chemotherapy at a cumulative dose of up to 500 mg/m² or in a dose of up to 100 mg/m² per cycle during one or more cycles of induction chemotherapy.

13. The method of claim 1, wherein the chimeric anti-GD2 antibody dinutuximab beta is administered to the patient as a continuous intravenous infusion over 24 hours per day, in a daily dose of 10 mg/m²; and/or wherein the chimeric anti-GD2 antibody dinutuximab beta is administered to the patient on consecutive days of a cycle until all of the dose per cycle of the chimeric anti-GD2 antibody dinutuximab beta has been administered; and/or wherein the administration of the chimeric anti-GD2 antibody dinutuximab beta improves one or more clinical parameters compared to the induction chemotherapy administered without the chimeric anti-GD2 antibody dinutuximab beta.

14. The method of claim 1 wherein the induction chemotherapy comprises 6 alternating chemotherapy cycles of:
cisplatin, etoposide, and vindesine; and
ifosfamide, vincristine, dacarbazine, and doxorubicin,
wherein the cycles are of at least 21 days.

15. The method of claim 1, wherein the induction chemotherapy comprises consecutive chemotherapy cycles of:
vincristine, carboplatin and etoposide;
vincristine and cisplatin;
vincristine, cyclophosphamide and etoposide;
vincristine and cisplatin;
vincristine, carboplatin and etoposide;
vincristine and cisplatin;
vincristine, cyclophosphamide and etoposide; and
vincristine and cisplatin,
wherein the cycles are of at least 10 days.

16. The method of claim 14, wherein the chimeric anti-GD2 antibody dinutuximab beta is administered to the patient at a cumulative dose of 160 to 380 mg/m² and/or a treatment density of 1.52 to 3.62 mg/m²/day.

17. The method of claim 15, wherein the chimeric anti-GD2 antibody dinutuximab beta is administered to the patient at a cumulative dose of 120 to 260 mg/m² and/or a treatment density of 2.00 to 4.30 mg/m²/day.

18. The method of claim 6, wherein the chimeric anti-GD2 antibody dinutuximab beta is administered to the patient during the induction chemotherapy at a treatment density of up to 5 mg/m²/day.

19. The method of claim 2, wherein the chimeric anti-GD2 antibody dinutuximab beta is administered to the patient as a continuous intravenous infusion over 24 hours per day, in a daily dose of 10 mg/m²; and wherein the chimeric anti-GD2 antibody dinutuximab beta is administered to the patient on consecutive days of a cycle until all of the dose per cycle of the chimeric anti-GD2 antibody dinutuximab beta has been administered; and wherein the administration of the chimeric anti-GD2 antibody dinutuximab beta improves one or more clinical parameters compared to the induction chemotherapy administered without the chimeric anti-GD2 antibody dinutuximab beta.

20. The method of claim 8, wherein the chimeric anti-GD2 antibody dinutuximab beta is administered to the patient as a continuous intravenous infusion over 24 hours per day, in a daily dose of 10 mg/m²; and wherein the chimeric anti-GD2 antibody dinutuximab beta is administered to the patient on consecutive days of a cycle until all of the dose per cycle of the chimeric anti-GD2 antibody dinutuximab beta has been administered; and wherein the administration of the chimeric anti-GD2 antibody dinutuximab beta improves one or more clinical parameters compared to the induction chemotherapy administered without the chimeric anti-GD2 antibody dinutuximab beta.
